(19) United States
(12) Patent Application Publication
(10) Pub. No.: US 2008/0112888 A1
(43) Pub. Date: May 15, 2008

(54) IGFBP2 BIOMARKER

(75) Inventor: Yan Wang, Scotch Plains, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/771,454

(22) Filed: Jun. 29, 2007

Related U.S. Application Data
(60) Provisional application No. 60/818,004, filed on Jun. 30, 2006.

(51) Int. Cl. A61K 31/505; A61K 49/00; A61P 37/00; A61P 43/00

(52) U.S. Cl. 424/9.2; 514/275

(57) ABSTRACT

The present invention provides method for quickly and conveniently determining if a given treatment regimen of IGF1R inhibitor is sufficient, e.g., to saturate IGF1R receptors in the body of a subject. Several clinically relevant determinations may be made based on this point, including, for example, whether the dosage of the regimen is sufficient or should be increased.

This application claims the benefit of U.S. provisional patent application No. 60/818,004; filed Jun. 30, 2006; which is herein incorporated by reference in its entirety.

### FIELD OF THE INVENTION

The present invention relates to methods for determining if an IGF1R inhibitor is efficacious in a patient receiving the inhibitor, for example, to treat cancer.

### BACKGROUND OF THE INVENTION

The insulin-like growth factors, also known as somatomedins, include insulin-like growth factor-I (IGF-I) and insulin-like growth factor-II (IGF-II) (Klapper, et al., (1983) Endocrinol. 112:2215 and Rinderknecht, et al., (1978) Febs. Lett. 89:283). These growth factors exert mitogenic activity on various cell types, including tumor cells (Macaulay, (1992) Br. J. Cancer 65:311), by binding to a common receptor named the insulin-like growth factor receptor-1 (IGF1R) (Sepp-Lorenzino, (1998) Breast Cancer Research and Treatment 47:235). Interaction of IGFs with IGF1R activates the receptor by triggering autophosphorylation of the receptor on tyrosine residues (Butler, et al., (1998) Comparative Biochemistry and Physiology 121:19). Once activated, IGF1R, can bind to intracellular substrates such as IRS-1 and Sch. Phosphorylated IRS-1 can activate the p85 regulatory subunit of PI3 kinase, leading to activation of several downstream substrates, including the p70 S6 kinase and protein kinase B (Akt). Akt phosphorylation in turn, enhances protein synthesis through mTOR activation and triggers anti-apoptotic effects of IGF1R through phosphorylation and inactivation of Bad. In parallel to PI3 kinase-driven signaling, recruitment of Grb2/SOS by phosphorylated IRS-1 or Shc leads to recruitment of Ras and activation of the Raf1/MEK/ERK pathway and downstream nuclear factors, resulting in the induction of cellular proliferation. Clearly, inhibition of the activity in this pathway would be a valuable means by which to treat diseases mediated by any member of this pathway (e.g., IGF1R). Inhibition of IGF1R activity has proven to be a valuable method to treat or prevent growth of human cancers and other proliferative diseases. For example, overexpression of insulin-like growth factor receptors-I has been demonstrated in several cancer cell lines and tumor tissues. Likewise, monitoring the activity of this pathway is a valuable marker for the effect of an IGF1R inhibitor on the pathway's downstream effects, e.g., malignant cell growth.

Another modulator of cellular growth is IGFBP2. IGFBP2 has been identified as a possible growth promoter of malignant cells. IGFBP2 expression depends, at least in part, on IGF1-mediated IGF1R activation (Martin et al., Endocrinology (2007) 148(5): 2532-2541). Activation of the PI3 kinase part of the IGF1R signaling pathway has been linked to IGFBP2 expression (Martin et al. (2007)).

Currently, there are several known anti-cancer therapies that target IGF1R; for example, anti-IGF1R antibodies (See e.g., WO2003/100008). Assessing the proper dosage to provide to a subject receiving IGF1R inhibitor therapy can be difficult using current technology. For example, a clinician may need to resort to measuring tumor size or cancer progression after several weeks or months of therapy so as to determine if the dosage is proper. Such a process can be time consuming and, thus, dangerous in view of the fact that certain cancers must be treated rapidly and effectively in order to reach a positive therapeutic outcome (e.g., survival). There is, thus, a need in the art for rapidly and conveniently determining whether a given dosage is proper.

### SUMMARY OF THE INVENTION

The present invention addresses this need by providing the methods of the present invention. As is discussed herein, the present invention provides a simple and convenient method for monitoring the magnitude or inhibition of the IGF1R system or cascade in the body of a subject receiving IGF1R inhibitor therapy by monitoring IGFBP2 levels in the subject's body over the course of inhibitor treatment. It has been demonstrated that IGF1R treatment causes IGFBP2 levels to depress over time in the body of a subject receiving the inhibitor. The link between IGFBP2 levels and the level of activity in the IGF1R signaling cascade make blood levels of IGFBP2 a convenient indicator for the magnitude of effect an inhibitor therapy is having on the cascade. It has also been demonstrated that IGFBP2 levels decrease by a maximum amount and that this amount represents a convenient pharmacokinetic target. A clinician or other practitioner administering an IGF1R inhibitor to a subject with a medical condition mediated by IGF1R can, in turn, follow blood IGFBP2 levels over time in the subject's body and, based on this observation, decide if treatment should be altered in some way, e.g., if dosage should be increased, decreased, maintained or discontinued.

For example, the present invention provides a method for monitoring the effect of an IGF1R inhibitor on IGFBP2 concentration in the body of a subject administered said inhibitor comprising measuring IGFBP2 levels in the body of the subject over time during a course of treatment of said inhibitor. Such clinical/pharmacokinetic data is valuable in the evaluation of both the efficacy and the dosage (e.g., amount and/or frequency) of an given IGF1R inhibitor therapeutic regimen. In a more specific embodiment of the present invention, the method comprises (i) measuring an IGFBP2 concentration in the body of said subject before treatment with said inhibitor (e.g., in a treatment-naïve subject never exposed to the inhibitor or in a subject who is in the midst of an ongoing therapeutic regimen); (ii) administering one or more doses of said inhibitor to said subject; (iii) measuring an IGFBP2 concentration in the body of said subject following said administration; (iv) comparing the level of IGFBP2 measured in step (i) with the level of IGFBP2 measured in step (iii). For example, inhibitor is determined to lower the IGFBP2 concentration if the level measured in step (i) is higher than the concentration measured in step (iii); and wherein the inhibitor is determined not to lower the IGFBP2 concentration if the level measured in step (i) is not higher than the concentration measured in step (iii).

The present invention provides a method for monitoring the effect of an IGF1R inhibitor on the IGF1R receptor in the body of a subject administered said inhibitor comprising evaluating IGFBP2 levels in the body of the subject over time; e.g., wherein the inhibitor is determined to inhibit the receptor if IGFBP2 levels are observed to decrease over time following said administration; or wherein the inhibitor is determined not to inhibit the receptor if IGFBP2 levels are not observed to decrease over time following said administration. In an embodiment of the invention, the inhibitor is determined to inhibit the receptor if IGFBP2 levels are observed to decrease by at least 51% over time following a first administration of said inhibitor; or wherein the inhibitor is determined not to inhibit the receptor if IGFBP2 levels are not observed to decrease by at least 51% over time following a first administration of said inhibitor. In an embodiment of the invention, the method comprises (i) measuring an IGFBP2 level in the body of said subject before treatment with said inhibitor; (ii) administering one or more doses of said inhibitor to said subject; (iii) measuring an IGFBP2 level in the body of said subject following said administration; (iv) comparing the level of IGFBP2 measured in step (i) with the level of IGFBP2 measured in step (iii); wherein the inhibitor is determined to inhibit the receptor if IGFBP2 levels are observed to decrease over time following said administration; or wherein the inhibitor is determined not to inhibit the receptor if IGFBP2 levels are not observed to decrease over time following said administration. In an embodiment of the invention, the IGF1R inhibitor is an antibody or antigen-binding fragment thereof that binds specifically to IGF1R.

The present invention also provides a method for evaluating dosage of an IGF1R inhibitor administered to a subject comprising administering a dose of said inhibitor to said subject and evaluating IGFBP2 levels in the body of the subject over time; wherein said dosage is determined to be insufficient if IGFBP2 levels are not observed to decrease by at least 51% over time following said administration; or wherein said dosage is determined to be sufficient if IGFBP2 levels are observed to decrease by at least 51% over time following said administration. In an embodiment of the invention, the method comprises (i) measuring an IGFBP2 level in the body of said subject before treatment with said inhibitor; (ii) administering one or more doses of said inhibitor to said subject; (iii) measuring an IGFBP2 level in the body of said subject following said administration; (iv) comparing the level of IGFBP2 measured in step (i) with the level of IGFBP2 measured in step (iii); wherein said dosage is determined to be insufficient if IGFBP2 levels are not observed to decrease by at least 51% over time following said administration; or wherein said dosage is determined to be sufficient if IGFBP2 levels are observed to decrease by at least 51% over time following said administration. For example, in an embodiment of the invention, if the dosage is determined to be acceptable, the subject is continued on a therapeutic regiment comprising administering the evaluated dose.

The present invention further comprises a method for determining if a subject has a medical condition that is responsive to an IGF1R inhibitor comprising administering said inhibitor to said subject and evaluating IGFBP2 levels in the body of the subject over time; wherein said condition is determined to be unresponsive to said inhibitor if the IGFBP2 levels are not observed to decrease over time following said administration. In an embodiment of the invention, the method comprises: (i) measuring an IGFBP2 level in the body of said subject before treatment with said inhibitor; (ii) administering one or more doses of said inhibitor to said subject; (iii) measuring an IGFBP2 level in the body of said subject following said administration; (iv) comparing the level of IGFBP2 measured in step (i) with the level of IGFBP2 measured in step (iii); wherein said condition is determined to be unresponsive to said inhibitor if the IGFBP2 levels are not observed to decrease over time following said administration. Also provided by the present invention is a method for determining if a subject has a medical condition that is responsive to an IGF1R inhibitor comprising administering said inhibitor to said subject and evaluating IGFBP2 levels in the body of the subject over time; wherein said condition is determined to be unresponsive to said inhibitor if the IGFBP2 levels are not observed to decrease over time following said administration. In an embodiment of the present invention, the method comprises: (i) measuring an IGFBP2 level in the body of said subject before treatment with said inhibitor; (ii) administering one or more doses of said inhibitor to said subject; (iii) measuring an IGFBP2 level in the body of said subject following said administration; (iv) comparing the level of IGFBP2 measured in step (i) with the level of IGFBP2 measured in step (iii); wherein said condition is determined to be unresponsive to said inhibitor if the IGFBP2 levels are not observed to decrease over time following said administration. For example, in an embodiment of the invention, the subject is administered a therapeutic regimen comprising administering the IGF1R inhibitor, e.g., at a dosage of 0.3, 1, 3, 10 or 20 mg/kg once a week.

The present invention also provides a method for treating a medical condition, in a subject, mediated by IGF1R expression or activity comprising: (i) measuring an IGFBP2 level in the body of said subject prior to any administration of an IGF1R inhibitor; (ii) administering one or more doses of an IGF1R inhibitor to said subject; (iii) measuring an IGFBP2 level in the body of said subject following said administration; (iv) comparing the level of IGFBP2 measured in step (i) with the level of IGFBP2 measured in step (iii); and (v) increasing dosage of said inhibitor if the IGFBP2 level does not decrease by at least 51% following said administration. In an embodiment of the invention, dosage is maintained if the 51% target is reached. In an embodiment of the invention, dosage is decreased if IGFBP2 levels are reduced significantly and unacceptably below 51%.

The present invention further provides a method for selecting a dose of an IGF1R inhibitor comprising administering a dose of said inhibitor to a subject with a medical condition mediated by IGF1R expression or activity and evaluating IGFBP2 levels in the body of the subject; wherein said dosage is selected if IGFBP2 levels are observed to decrease by at least 51% of an IGFBP2 level measured prior to first administration of said inhibitor following said administration. For example, in an embodiment of the invention, the method comprises (i) measuring an IGFBP2 level in the body of said subject before treatment with said inhibitor; (ii) administering one or more doses of said inhibitor to said subject (e.g., wherein the doses are of a common amount and frequency); (iii) measuring an IGFBP2 level in the body of said subject following said administration; and (iv) comparing the level of IGFBP2 measured in step (i) with the level of IGFBP2 measured in step (iii); wherein said dose is selected if IGFBP2 levels are observed to decrease by at least 51% of an IGFBP2 level measured prior to first administration of said inhibitor following said administration. For example, in an embodiment of the invention, a therapeutic regimen comprising administration of the dose is continued if the dose is selected.

In an embodiment of any of the inventions discussed herein, the IGF1R inhibitor is an antibody or antigen-binding fragment thereof that binds specifically to IGF1R, e.g., wherein the antibody or fragment comprises one or more complementarity determining regions (CDRs) selected from the group consisting of:
RASQSIGSSLH (SEQ ID NO:) e.g., which is CDR-L1;
YASQSLS (SEQ ID NO:) e.g., which is CDR-L2;
HQSSRLPHT (SEQ ID NO:) e.g., which is CDR-L3;
SFAMH (SEQ ID NO:) e.g., which is CDR-H1
GFTFSSFAMH (SEQ ID NO:) e.g., which is CDR-H1;
VIDTRGATYYADSVKG (SEQ ID NO:) e.g., which is CDR-H2;
LGNFYYGMDV (SEQ ID NO:) e.g., which is CDR-H3;
   or a mature fragment of a light chain immunoglobulin which comprises the amino acid sequence of SEQ ID NO: 2, 4, 6 or 8; or a mature fragment of a heavy chain immunoglobulin which comprises the amino acid sequence of SEQ ID NO: 10 or 12; or a pharmaceutical composition thereof which comprises a pharmaceutically acceptable carrier.

In an embodiment of any of the inventions discussed herein the subject suffers from a medical disorder mediated by IGF1R expression or activity, e.g., osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

In an embodiment of any of the inventions discussed herein, the subject is also administered one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 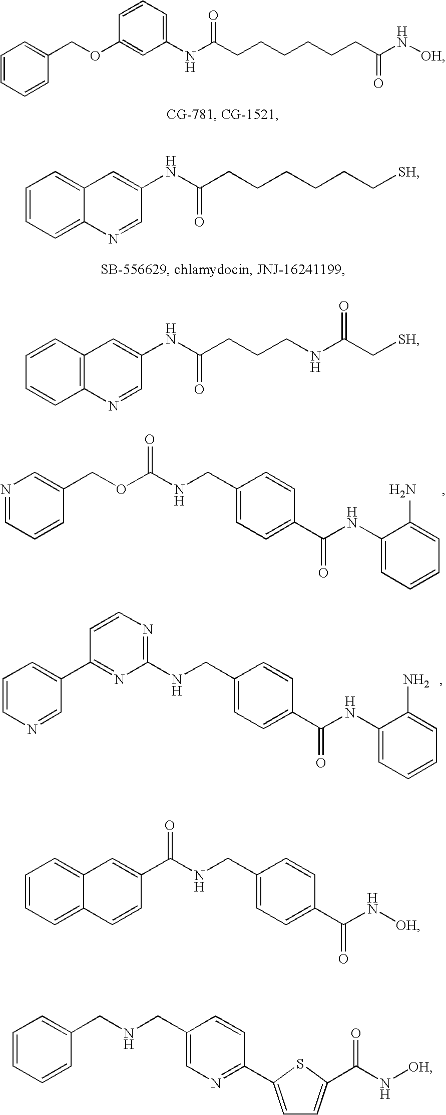
vorinostat, etoposide, gemcitabinc, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258, 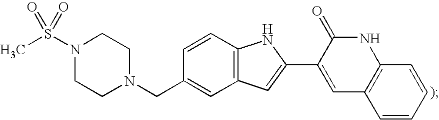
3-[5-(methylsulfonylpiperadinemethyl)-indolyl]-quinolone, vatalanib, AG-013736, AVE-0005, the acetate salt of [D-Ser(Bu t) 6, Azgly 10](pyro-Glu-His-Trp-Ser-Tyr-D-Ser(Bu t)-Leu-Arg-Pro-Azgly-NH₂ acetate [C₅₉He₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165 HKI-272, erlotinib, lapatanib, canertinib, ABX-EGF antibody, erbitux, EKB-569, PKI-166, GW-572016, lonafarnib, 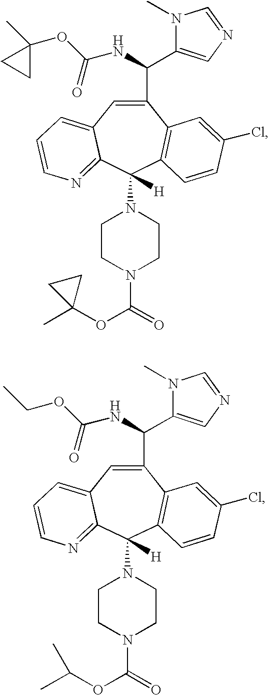
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, trelinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa. In an embodiment of any of the inventions discussed herein the IGFBP2 level is determined using a radioimmunoassay (RIA), a western blot or an enzyme linked immunosorbent assay (ELISA) of a sample from the patient. Also, in an embodiment of any of the inventions discussed herein the sample which is evaluated for IGFBP2 concentration is blood or plasma from the patient.

### DETAILED DESCRIPTION OF THE INVENTION

As is discussed herein, IGFBP2 levels have demonstrated to be a very useful pharmacokinetic marker for the magnitude by which an IGF1R inhibitor is inhibiting the IGF1R pathway in cells (e.g., malignant cells) in a subject's body. This data provides information which is valuable to a clinician in evaluating the appropriateness of a given dosage of the inhibitor. If, in view of the IGFBP2 levels observed, the IGF1R pathway is deemed, in the clinician's expert judgment, not to be sufficiently inhibited, then the dosage may be increased. If dosage and inhibition of the pathway is deemed sufficient, then dosage may be maintained. If dosage in pathway inhibition is deemed to be too high, dosage may be reduced.

It has been determined that the point at which IGFBP2 levels decrease in the blood of a subject by at least 51% (e.g., at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) during course of an IGF1R inhibitor (e.g., an anti-IGF1R antibody as discussed herein) treatment regimen, receptors in the body of the subject are essentially saturated with the inhibitor. This point makes 51% a very useful pharmacokinetic target in the treatment of any disease mediated by IGF1R expression or activity with an IGF1R inhibitor. The 51% target or any of the target percentages discussed above (e.g., at least 60%, at least 70%, etc.) may be used at the target in connection with any of the methods discussed herein. Any such embodiment forms part of the present invention.

Furthermore, an aspect of the invention includes determining whether a patient exhibits elevated IGFBP2 levels. As discussed herein, the IGFBP2 levels in a patient has been correlated to tumor size; higher levels of IGFBP2 correlate to a large tumor size and vice versa.

The terms insulin-like growth factor-binding protein 2, IGFBP-2, IBP-2 or IGF-binding protein 2 are well known in the art. In an embodiment of the invention, an IGFBP2 is human, e.g., which comprises the following amino acid sequence:

(SEQ ID NO:105)

MLPRVGCPAL PLPPPPLLPL LPLLLLLLGA SGGGGGARAE
VLFRCPPCTP ERLAACGPPP VAPPAAVAAV AGGARMPCAE
LVREPGCGCC SVCARLEGEA CGVYTPRCGQ GLRCYPHPGS
ELPLQALVMG EGTCEKRRDA EYGASPEQVA DNGDDHSEGG
LVENHVDSTM NMLGGGGSAG RKPLKSGMKE LAVFREKVTE
QHRQMGKGGK HHLGLEEPKK LRPPPARTPC QQELDQVLER
ISTMRLPDER GPLEHLYSLH IPNCDKHGLY NLKQCKMSLN
GQRGECWCVN PNTGKLIQGA PTIRGDPECH LFYNEQQEAR
GVHTQRMQ

See also UniProtKB/Swiss-Prot accession no. P18065, Genbank accession no. NP_{—}000588; accession nos. IPI00297284.1 and M35410 and EMBL accession nos. A09809.

The term “IGF1R” or “insulin-like growth factor-1 receptor”, or the like, includes any species of IGF1R, e.g., human IGF1R.

In an embodiment, an antibody or antigen-binding fragment thereof that binds “specifically” to IGF1R (e.g., human IGF1R) binds with a Kd of about 10⁻⁸ M or 10⁻⁷ M or a lower number; or, in an embodiment of the invention, with a Kd of about 1.28×10⁻¹⁰ M or a lower number by Biacore measurement or with a Kd of about 2.05×10⁻¹² or a lower number by KinExA measurement. In another embodiment, an antibody that binds “specifically” to human IGF1R binds exclusively to human IGF1R and to no other protein (e.g., non-human IGF1R).

### IGF1R Inhibitors

The terms “IGF1R inhibitor” or “IGF1R antagonist” or the like include any substance that decreases the expression, ligand binding (e.g., binding to IGF-1 and/or IGF-2), kinase activity (e.g., autophosphorylation activity) or any other biological activity of IGF1R (e.g., mediation of anchorage independent cellular growth) that will elicit a biological or medical response of a tissue, system, subject or patient that is being sought by the administrator (such as a researcher, doctor or veterinarian) which includes any measurable alleviation of the signs, symptoms and/or clinical indicia of cancer (e.g., tumor growth) and/or the prevention, slowing or halting of progression or metastasis of cancer (e.g., neuroblastoma) to any degree.

In an embodiment of the invention, an IGF1R inhibitor that is administered to a patient in a method according to the invention is any isolated antibody or antigen-binding fragment thereof that binds specifically to insulin-like growth factor-1 receptor (e.g., human IGF1R) or any soluble fragment thereof (e.g., monoclonal antibodies (e.g., fully human monoclonal antibodies), polyclonal antibodies, bispecific antibodies, Fab antibody fragments, F(ab)₂ antibody fragments, Fv antibody fragments (e.g., VH or VL), single chain Fv antibody fragments, dsFv antibody fragments, humanized antibodies, chimeric antibodies or anti-idiotypic antibodies) such as any of those disclosed in any of Burtrum et. al Cancer Research 63:8912-8921(2003); in French Patent Applications FR2834990, FR2834991 and FR2834900 and in PCT Application Publication Nos. WO 03/100008; WO 03/59951; WO 04/71529; WO 03/106621; WO 04/83248; WO 04/87756, WO 05/16970; and WO 02/53596.

In an embodiment of the invention, an IGF1R inhibitor that is administered to a patient in a method according to the invention is an isolated anti-insulin-like growth factor-1 receptor (IGF1R) antibody comprising a mature 19D12/15H12 Light Chain (LC)-C, D, E or F and a mature 19D12/15H12 heavy chain (HC)-A or B (e.g., mature LCF/mature HCA). In an embodiment of the invention, an IGF1R inhibitor that is administered to a patient in a method according to the invention is an isolated antibody that specifically binds to IGF1R that comprises one or more complementarity determining regions (CDRs) of 19D12/15H12 Light Chain-C, D, E or F and/or 19D12/15H12 heavy chain-A or B (e.g., all 3 light chain CDRs and all 3 heavy chain CDRs).

The amino acid and nucleotide sequences of the some antibody chains of the invention are shown below. Dotted, underscored type indicates the signal peptide. Solid underscored type indicates the CDRs. Plain type indicates the framework regions. Mature or processed fragments lack the signal peptide (such mature fragments and antibodies or antigen-binding fragments thereof including such mature fragments form part of the present invention along with their uses).

Modified 19D12/15H12 Light Chain-C
(SEQ ID NO:1)
ATG TCG CCA TCA CAA CTC ATT GGG TTT CTG CTG CTC TGG GTT CCA GCC TCC
-------------------------------------------------------------------
AGG GGT GAA ATT GTG CTG ACT CAG AGC CCA GAC TCT CTG TCT GTG ACT CCA
-------
GGC GAG AGA GTC ACC ATC ACC TGC CGG GCC AGT CAG AGC ATT GGT AGT AGC
TTA CAC TGG TAC CAG CAG AAA CCA GGT CAG TCT CCA AAG CTT CTC ATC AAG
TAT GCA TCC CAG TCC CTC TCA GGG GTC CCC TCG AGG TTC AGT GGC AGT GGA
TCT GGG ACA GAT TTC ACC CTC ACC ATC AGT AGC CTC GAG GCT GAA GAT GCT
GCA GCG TAT TAC TGT CAT CAG AGT AGT CGT TTA CCT CAC ACT TTC GGC CAA
GCG ACC AAG GTG GAG ATC AAA CGT ACG
(SEQ ID NO:2)
M S P S Q L I G F L L L W V P A S
-----------------------------------------------------------------
R G E I V L T Q S P D S L S V T P
-----
G E R V T I T C R A S Q S I G S S
L H W Y Q Q K P G Q S P K L L I K
Y A S Q S L S G V P S R F S G S G
S G T D F T L T I S S L E A E D A
A A Y Y C H Q S S R L P H T F G Q
G T K V E I K R T
Modified 19D12/15H12 Light Chain-D
(SEQ ID NO:3)
ATG TCG CCA TCA CAA CTC ATT GGG TTT CTG CTG CTC TGG GTT CCA GCC TCC
-------------------------------------------------------------------
AGG GGT GAA ATT GTG CTG ACT CAG AGC CCA GAC TCT CTG TCT GTG ACT CCA
-------
GGC GAG AGA GTC ACC ATC ACC TGC CGG GCC AGT CAG AGC ATT GGT AGT AGC
TTA CAC TGG TAC CAG CAG AAA CCA GGT CAG TCT CCA AAG CTT CTC ATC AAG
TAT GCA TCC CAG TCC CTC TCA GGG GTC CCC TCG AGG TTC AGT GGC AGT GGA
TCT GGG ACA GAT TTC ACC CTC ACC ATC AGT AGC CTC GAG GCT GAA GAT TTC
GCA GTG TAT TAC TGT CAT CAG AGT AGT CGT TTA CCT CAC ACT TTC GGC CAA
GGG ACC AAG GTG GAG ATC AAA CGT ACG
(SEQ ID NO:4)
M S P S Q L I G F L L L W V P A S
-----------------------------------------------------------------
R G E I V L T Q S P D S L S V T P
-----
G E R V T I T C R A S Q S I G S S
L H W Y Q Q K P G Q S P K L L I K
Y A S Q S L S G V P S R F S G S G
S G T D F T L T I S S L E A E D F
A V Y Y C H Q S S R L P H T F G Q
G T K V E I K R T
Modified 19D12/15H12 Light Chain-E
(SEQ ID NO:5)
ATG TCG CCA TCA CAA CTC ATT GGG TTT CTG CTG CTC TGG GTT CCA GCC TCC
-------------------------------------------------------------------
AGG GGT GAA ATT GTG CTG ACT CAG AGC CCA GGT ACC CTG TCT GTG TCT CCA
-------
GGC GAG AGA GCC ACC CTC TCC TGC CGG GCC AGT CAG AGC ATT GGT AGT AGC
TTA CAC TGG TAC CAG CAG AAA CCA GGT CAG GCT CCA AGG CTT CTC ATC AAG
-------
TAT GCA TCC CAG TCC CTC TCA GGG ATC CCC GAT AGG TTC AGT GGC AGT GGA
TCT GGG ACA GAT TTC ACC CTC ACC ATC AGT AGA CTG GAG CCT GAA GAT GCT
GCA GCG TAT TAC TGT CAT CAG AGT AGT CGT TTA CCT CAC ACT TTC GGC CAA
GGG ACC AAG GTG GAG ATC AAA CGT ACA
(SEQ ID NO:6)
M S P S Q L I G F L L L W V P A S
-----------------------------------------------------------------
R G E I V L T Q S P G T L S V S P
-----
G E R A T L S C R A S Q S I G S S
L H W Y Q Q K P G Q A P R L L I K
Y A S Q S L S G I P D R F S G S G
S G T D F T L T I S R L E P E D A
A A Y Y C H Q S S R L P H T F G Q
G T K V E I K R T
Modified 19D12/15H12 Light Chain-F
(SEQ ID NO:7)
ATG TCG CCA TCA CAA CTC ATT GGG TTT CTG CTG CTC TGG GTT CCA GCC TCC
-------------------------------------------------------------------
AGG GGT GAA ATT GTG CTG ACT CAG AGC CCA GGT ACC CTG TCT GTG TCT CCA
-------
GGC GAG AGA GCC ACC CTC TCC TGC CGG GCC AGT CAG AGC ATT GGT AGT AGC
TTA CAC TGG TAC CAG CAG AAA CCA GGT CAG GCT CCA AGG CTT CTC ATC AAG
TAT GCA TCC CAG TCC CTC TCA GGG ATC CCC GAT AGG TTC AGT GGC AGT GGA
TCT GGG ACA GAT TTC ACC CTC ACC ATC AGT AGA CTG GAG CCT GAA GAT TTC
GCA GTG TAT TAC TGT CAT CAG AGT AGT CGT TTA CCT CAC ACT TTC GGC CAA
GGG ACC AAG GTG GAG ATC AAA CGT ACA
(SEQ ID NO:8)
M S P S Q L I G F L L L W V P A S
-----------------------------------------------------------------
R G E I V L T Q S P G T L S V S P
-----
G E R A T L S C R A S Q S I G S S
L H W Y Q Q K P G Q A P R L L I K
Y A S Q S L S G I P D R F S G S G
S G T D F T L T I S R L E P E D F
A V Y Y C H Q S S R L P H T F G Q
G T K V E I K R T
Modified 19D12/15H12 heavy chain-A
(SEQ ID NO:9)
ATG GAG TTT GGG CTG AGC TGG GTT TTC CTT GTT GCT ATA TTA AAA GGT GTC
-------------------------------------------------------------------
CAG TGT GAG GTT CAG CTG GTG CAG TCT GGG GGA GGC TTG GTA AAG CCT GGG
-------
GGG TCC CTG AGA CTC TCC TGT GCA GCC TCT GGA TTC ACC TTC AGT AGC TTT
GCT AGT CAC TGG GTT CGC CAG GCT CCA GGA AAA GGT CTG GAG TGG ATA TCA
GTT ATT GAT ACT CGT GGT GCC ACA TAC TAT GCA GAC TCC GTG AAG GGC CGA
TTC ACC ATC TCC AGA GAC AAT GCC AAG AAC TCC TTG TAT CTT CAA ATG AAC
AGC CTG AGA GCC GAG GAC ACT GCT GTG TAT TAC TGT GCA AGA CTG GGG AAC
TTC TAC TAC GGT ATG GAC GTC TGG GGC CAA GGG ACC ACG GTC ACC GTC TCC
TCA
(SEQ ID NO:10)
Met Glu Phe Gly Leu Ser Trp Val Phe Leu Val Ala Ile Leu Lys Gly Val
-------------------------------------------------------------------
Gln Cys Glu Val Gln Leu Val Gln Ser Gly Gly Gly Leu Val Lys Pro Gly
-------
Gly Ser Leu Arg Leu Ser Cys Ala Ala Ser Gly Phe Thr Phe Ser Ser Phe
Ala Met His Trp Val Arg Gln Ala Pro Gly Lys Gly Leu Glu Trp Ile Ser
Val Ile Asp Thr Arg Gly Ala Thr Tyr Tyr Ala Asp Ser Val Lys Gly Arg
Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Leu Gly Asn
Phe Tyr Tyr Gly Met Asp Val Trp Gly Gln Gly Thr Thr Val Thr Val Ser
Ser
Modified 19D12/15H12 heavy chain-B
(SEQ ID NO:11)
ATG GAG TTT GGG CTG AGC TGG GTT TTC CTT GTT GCT ATA TTA AAA GGT GTC
-------------------------------------------------------------------
CAG TGT GAG GTT CAG CTG GTG CAG TCT GGG GGA GGC TTG GTA CAG CCC GGG
-------
GGG TCC CTG AGA CTC TCC TGT GCA GCC TCT GGA TTC ACC TTC AGT AGC TTT
GCT ATG CAC TGG GTT CGC CAG GCT CCA GGA AAA GGT CTG GAG TGG ATA TCA
GTT ATT GAT ACT CGT GGT GCC ACA TAC TAT GCA GAC TCC GTG AAG GGC CGA
TTC ACC ATC TCC AGA GAC AAT GCC AAG AAC TCC TTG TAT CTT CAA ATG AAC
AGC CTG AGA GCC GAG GAC ACT GCT GTG TAT TAC TGT GCA AGA CTG GGG AAC
TTC TAC TAC GGT ATG GAC GTC TGG GGC CAA GGG ACC ACG GTC ACC GTC TCC
TCA
(SEQ ID NO:12)
Met Glu Phe Gly Leu Ser Trp Val Phe Leu Val Ala Ile Leu Lys Gly Val
-------------------------------------------------------------------
Gln Cys Glu Val Gln Leu Val Gln Ser Gly Gly Gly Leu Val Gln Pro Gly
-------
Gly Ser Leu Arg Leu Ser Cys Ala Ala Ser Gly Phe Thr Phe Ser Ser Phe
Ala Met His Trp Val Arg Gln Ala Pro Gly Lys Gly Leu Glu Trp Ile Ser
Val Ile Asp Thr Arg Gly Ala Thr Tyr Tyr Ala Asp Ser Val Lys Gly Arg
Phe Thr Ile Ser Arg Asp Asn Ala Lys Asn Ser Leu Tyr Leu Gln Met Asn
Ser Leu Arg Ala Glu Asp Thr Ala Val Tyr Tyr Cys Ala Arg Leu Gly Asn
Phe Tyr Tyr Gly Met Asp Val Trp Gly Gln Gly Thr Thr Val Thr Val Ser
Ser

Cell lines containing plasmids comprising a CMV promoter operably linked to the 15H12/19D12 light chains and heavy chains have been deposited at the American Type Culture Collection (ATCC); 10801 University Boulevard; Manassas, Va. 20110-2209 on May 21, 2003. The deposit name and the ATCC accession numbers for the cell lines are set forth below:
CMV promoter-15H12/19D12 LCC (κ)-

Deposit name: “15H12/19D12 LCC (κ)”;

ATCC accession No.: PTA-5217
CMV promoter-15H12/19D12 LCD (κ)-

Deposit name: “15H12/19D12 LCD (κ)”;

ATCC accession No.: PTA-5218
CMV promoter-15H12/19D12 LCE (κ)-

Deposit name: “15H12/19D12 LCE (κ)”;

ATCC accession No.: PTA-5219
CMV promoter-15H12/19D12 LCF (κ)-

Deposit name: 15H12/19D12 LCF (κ)”;

ATCC accession No.: PTA-5220
CMV promoter-15H12/19D12 HCA (γ4)-

Deposit name: “15H12/19D12 HCA (γ4)”

ATCC accession No.: PTA-5214
CMV promoter-15H12/19D12 HCB (γ4)-

Deposit name: “15H12/19D12 HCB (γ4)”

ATCC accession No.: PTA-5215
CMV promoter-15H12/19D12 HCA (γ1)-

Deposit name: “15H12/19D12 HCA (γ1)”;

ATCC accession No.: PTA-5216

All restrictions on access to the cell lines deposited in ATCC will be removed upon grant of a patent. The present invention includes methods and compositions (e.g., any disclosed herein) comprising anti-IGF1R antibodies and antigen-binding fragments thereof comprising any of the light and/or heavy immunoglobulin chains or mature fragments thereof located in any of the foregoing plasmids deposited at the ATCC.

In an embodiment of the invention, the IGF1R inhibitor is an isolated antibody or antigen-binding fragment thereof comprising one or more (e.g., 3) of the following CDR sequences:

| | |
|---|---|
| RASQSIGSSLH; | (SEQ ID NO:99) |
| YASQSLS; | (SEQ ID NO:100) |
| HQSSRLPHT; | (SEQ ID NO:101) |
| SFAMH; | (SEQ ID NO:102) |
| VIDTRGATYYADSVKG; | (SEQ ID NO:103) |
| LGNFYYGMDV. | (SEQ ID NO:104) |

For example, in an embodiment of the invention, a light chain immunoglobulin comprises 3 CDRs and/or a heavy chain immunoglobulin comprises 3 CDRs.

In an embodiment, an antibody that binds “specifically” to human IGF1R binds with a Kd of about 10⁻⁸ M or 10⁻⁷ M or a lower number; or, in an embodiment of the invention, with a Kd of about 1.28×10⁻¹⁰ M or a lower number by Biacore measurement or with a Kd of about 2.05×10⁻¹² or a lower number by KinExA measurement. In another embodiment, an antibody that binds “specifically” to human IGF1R binds exclusively to human IGF1R and to no other protein at significant or at detectable levels.

In an embodiment of the invention, an IGF1R inhibitor that is administered to a patient in a method according to the invention comprises any light chain immunoglobulin and/or a heavy chain immunoglobulin as set forth in Published International Application No. WO 2002/53596 which is herein incorporated by reference in its entirety. For example, in an embodiment, the antibody comprises a light chain variable region comprising an amino acid sequence selected from the group consisting of SEQ ID NOs: 2, 6, 10, 14, 18, 22, 47 and 51 as set forth in WO 2002/53596 and/or a heavy chain variable region comprising an amino acid sequence selected from the group consisting of SEQ ID NOs: 4, 8, 12, 16, 20, 24, 45 and 49 as set forth in WO 2002/53596. In an embodiment, the antibody comprises a heavy and/or light chain selected from that of antibody 2.12.1; 2.13.2; 2.14.3; 3.1.1; 4.9.2; and 4.17.3 in WO 2002/53596.

In an embodiment of the invention, an IGF1R inhibitor that can be administered to a patient in a method according to the invention comprises any light chain immunoglobulin and/or a heavy chain immunoglobulin as set forth in Published International Application No. WO 2003/59951 which is herein incorporated by reference in its entirety. For example, in an embodiment, the antibody comprises a light chain variable region comprising an amino acid sequence selected from the group consisting of SEQ ID NOs: 54, 61 and 65 as set forth in WO 2003/59951 and/or a heavy chain variable region comprising an amino acids sequence selected from the group consisting of SEQ ID NOs: 69, 75, 79 and 83 as set forth in WO 2003/59951.

In an embodiment of the invention, an IGF1R inhibitor that can be administered to a patient in a method according to the invention comprises any light chain immunoglobulin and/or a heavy chain immunoglobulin as set forth in Published International Application No. WO 2004/83248 which is herein incorporated by reference in its entirety. For example, in an embodiment, the antibody comprises a light chain variable region comprising an amino acid sequence selected from the group consisting of SEQ ID NOs: 109, 111, 113, 115, 117, 119, 121, 123, 125, 127, 129, 131, 133, 135, 137, 139, 141 and 143 as set forth in WO 2004/83248 and/or a heavy chain variable region comprising an amino acids sequence selected from the group consisting of SEQ ID NOs: 108, 110, 112, 114, 116, 118, 120, 122, 124, 126, 128, 130,132, 134, 136, 138, 140 and 142 as set forth in WO 2004/83248. In an embodiment, the antibody comprises a light and/or heavy chain selected from that of PINT-6A1; PINT-7A2; PINT-7A4; PINT-7A5; PINT-7A6; PINT-8A1; PINT-9A2; PINT-11A1; PINT-11A2; PINT-11A3; PINT-11A4; PINT-11A5; PINT-11A7; PINT-12A1; PINT-12A2; PINT-12A3; PINT-12A4 and PINT-12A5 in WO 2004/83248.

In an embodiment of the invention, an IGF1R inhibitor that can be administered to a patient in a method according to the invention comprises any light chain immunoglobulin and/or a heavy chain immunoglobulin as set forth in Published International Application No. WO 2003/106621 which is herein incorporated by reference in its entirety. For example, in an embodiment, the antibody comprises a light chain variable region comprising an amino acid sequence selected from the group consisting of SEQ ID NOs: 8-12, 58-69, 82-86, 90, 94, 96, 98, as set forth in WO 2003/106621 and/or a heavy chain variable region comprising an amino acids sequence selected from the group consisting of SEQ ID NOs: 7, 13, 70-81, 87, 88, 92 as set forth in WO 2003/106621.

In an embodiment of the invention, an IGF1R inhibitor that can be administered to a patient in a method according to the invention comprises any light chain immunoglobulin and/or a heavy chain immunoglobulin as set forth in Published International Application No. WO 2004/87756 which is herein incorporated by reference in its entirety. For example, in an embodiment, the antibody comprises a light chain variable region comprising an amino acid sequence of SEQ ID NO: 2 as set forth in WO 2004/87756 and/or a heavy chain variable region comprising an amino acid sequence of SEQ ID NO: 1 as set forth in WO 2004/87756.

In an embodiment of the invention, an IGF1R inhibitor that can be administered to a patient in a method according to the invention comprises any light chain immunoglobulin and/or a heavy chain immunoglobulin as set forth in Published International Application No. WO 2005/16970 which is herein incorporated by reference in its entirety. For example, in an embodiment, the antibody comprises a light chain variable region comprising an amino acid sequence of SEQ ID NO: 6 or 10 as set forth in WO 2005/16970 and/or a heavy chain variable region comprising an amino acid sequence of SEQ ID NO: 2 as set forth in WO 2005/16970.

In an embodiment of the invention, an anti-IGF1R antibody or antigen-binding fragment thereof of the invention comprises an immunoglobulin heavy chain variable region comprising an amino acid sequence selected from the group consisting of:

```
(SEQ ID NO:13)
    1 grlgqawrsl rlscaasgft fsdyymswir qapgkglewv syisssgstr
  51 dyadsvkgrf tisrdnakns lylqmnslra edtavyycvr dgvetttyyy
101 yygmdvwgqg ttvtvssast kgpsvfplap csrstsesta algclvkdyf
151 pepvtvswns galtsgvhtf psca
(SEQ ID NO:14)
    1 vqllesgggl vqpggslrls ctasgftfss yamnwvrqap gkglewvsai
  51 sgsggttfya dsvkgrftis rdnsrttlyl qmnslraedt avyycakdlg
101 wsdsyyyyyg mdvwgqgttv tvss
(SEQ ID NO:15)
    1 gpglvkpset lsltctvsgg sisnyywswi rqpagkglew igriycsgsp
  51 nynpslksrv tmsvdtsknq fslklnsvta adtavyycav tifgvviifd
101 ywgqgtlvtv ss
(SEQ ID NO:16)
    1 evqllesggg ivqpggslrl scaasgftfs syamswvrqa pgkglewvsa
  51 isgsggityy adsvkgrfti srdnskntly lqmnslraed tavyycakdl
101 gygdfyyyyy gmdvwgqgtt vtvss
(SEQ ID NO:17)
    1 pglvkpsetl sltctvsggs issyywswir qppgkglewi gyiyysgstn
  51 ynpslksrvt isvdtsknqf slklssvtaa dtavyycart ysssfyyygm
101 dvwgqgttvt vss
(SEQ ID NO:18)
    1 evqllesggg lvqpggslrl scaasgftfs syamswvrqa pgkglewvsg
  51 itgsggstyy adsvkgrfti srdnskntly lqmnslraed tavyycakdp
101 gttvimswfd pwgqgtlvtv ss
```

In an embodiment of the invention, an anti-IGF1R antibody or antigen-binding fragment thereof of the invention comprises an immunoglobulin light chain variable region comprising an amino acid sequence selected from the group consisting of:

```
(SEQ ID NO:19)
    1 asvgdrvtft crasqdirrd lgwyqqkpgk apkrliyaas rlqsgvpsrf
  51 sgsgsgteft ltisslqped fatyyclqhn nyprttgqgt eveiirtvaa
101 psvfifppsd eqlksgtasv vcllnnfypr eakvqw
(SEQ ID NO:20)
    1 diqmtqtpss lsasvgdrvt itcrasqgir ndlgwyqqkp gkapkrliya
  51 asrlhrgvps rfsgsgsgte ftltisslqp edfatyyclq hnsypcsfgq
101 gtkleik
(SEQ ID NO:21)
    1 sslsasvgdr vtftcrasqd irrdlgwyqq kpgkapkrli yaasrlqsgv
  51 psrfsysgsg teftltissl qpedfatyyc lqhnnyprtf gqgteveiir
(SEQ ID NO:22)
    1 diqmtqspss lsasvgdrvt itcrasqgir sdlgwfqqkp gkapkrliya
  51 asklhrgvps rfsgsgsgte ftltisrlqp edfatyyclq hnsypltfgg
101 gtkveik
(SEQ ID NO:23)
    1 gdrvtitcra sqsistflnw yqqkpgkapk llihvasslq ggvpsrfsgs
  51 gsgtdftlti sslqpedfat yycqqsynap ltfgggtkve ik
(SEQ ID NO:24)
    1 ratlscrasq svrgrylawy qqkpgqaprl liygassrat gipdrfsgsg
    51 sgtdftltis rlepedfavf ycqqygsspr tfgqgtkvei k
```

In an embodiment of the invention, the anti-IGF1R antibody comprises a light chain immunoglobulin, or a mature fragment thereof (i.e., lacking signal sequence), or variable region thereof, comprising the amino acid sequence of:

```
(SEQ ID NO:25)
    1 mdmrvpaqll qllllwfpga rcdiqmtqsp sslsasvgdr vtitc
  51 wyqq kpgkapkrli ygv psrfsgsgsg teftltissl
101 qpedfatyyc f gqgtkveikr tvaapsvfif ppsdeqlksg
151 tasvvcllnn fypreakvpq kvdnalqsgn sqesvteqds kdstyslsst
201 ltlskadyek hkvyacevth qglsspvtks fnrgec;
(SEQ ID NO:26)
    1 mdmrvpaqll gllllwfpga rcdiqmtqsp sslsasvgdr vtftc
  51 wyqq kpgkapkrli ygv psrfsgsgsg teftltissl
101 qpedfatyyc f gqgteveiir tvaapsvfif ppsdeqlksg
151 tasvvcllnn fypreakvqw kvdnalqsgn sqesvteqds kdstyslsst
201 ltlskadyek hkvyacevth qglsspvtks fnrgec;
(SEQ ID NO:27)
    1 mdmrvpaqll gllllwfpga rcdiqmtqsp sslsasvgdr vtitc
  51 wyqq kpgkapkrli ygv psrfsgsgsg teftltissl
101 qpedfatyyc f gqgtkleikr tvaapsvfif ppsdeqlksg
151 tasvvcllnn fypreakvqw kvdnalqsgn sqesvteqds kdstyslsst
201 ltlskadyek hkvyacevth qglsspvtks fnrgec;
or
(SEQ ID NO:28)
    1 mdmrvpaqll gllllwfpga rcdiqmtqfp sslsasvgdr vtitc
  51 wyqq kpgkapkrli ygv psrfsgsgsg teftltissl
101 qpedfatyyc f gqgtkleikr tvaapsvfif ppsdeqlksg
151 tasvvcllnn fypreakvqw kvdnalqsgn sqesvteqds kdstyslsst
201 ltlskadyek hkvyacevth gglsspvtks fnrgec.
```

In an embodiment of the invention, the signal sequence is amino acids 1-22 of SEQ ID NOs: 25-28, In an embodiment of the invention, the mature variable region is underscored. In an embodiment of the invention, the CDRs are in bold/italicized font. In an embodiment of the invention, the anti-IGF1R antibody or antigen-binding fragment thereof of the invention comprises one or more CDRs (e.g., 3 light chain CDRS) as set forth above.

In an embodiment of the invention, the anti-IGF1R antibody comprises a heavy chain immunoglobulin or a mature fragment thereof (i.e., lacking signal sequence), or a variable region thereof, comprising the amino acid sequence of:

```
(SEQ ID NO:29)
    1 mefglswvfl vaiikgvqcq vqlvesgggl vkpggslrls caas
  51 wirqap gkglewvsrftis rdnaknslyl
101 qmnslraedt avyycarwgqg ttvtvssast
151 kgpsvfplap csrstsesta algclvkdyf pepvtvswns galtsgvhtf
201 pavlqssgly slssvvtvps snfgtqtytc nvdhkpsntk vdktverkcc
251 vecppcpapp vagpsvflfp pkpkdtlmis rtpevtcvvv dvshedpevq
301 fnwyvdgvev hnaktkpree qfnstfrvvs vltvvhqdwl ngkeykckvs
351 nkglpapiek tisktkgqpr epqvytlpps reemtknqvs ltclvkgfyp
401 sdiavewesn gqpennyktt ppmldsdgsf flyskltvdk srwqqgnvfs
451 csvmhealhn hytqkslsls pgk;
(SEQ ID NO:30)
    1 mefgkswvfl vaiikgvqcq aqlvesgggl vkpggslrls caas
  51 wirqap gkglewvsrftis rdnaknslyl
101 qmnslraedt avyycvrwgqgttv tvssastkgp
151 svfplapcsr stsescaalg clvkdyfpep vtvswnsgal tsgvhtfpav
201 lqssglysls svvtvpssnf gtqtytcnvd hkpsntkvdk tverkccvec
251 ppcpappvag psvflfppkp kdtlmisrtp evtcvvvdvs hedpevqfnw
301 yvdgvevhna ktkpreeqfn stfrvvsvlt vvhqdwlngk eykckvsnkg
351 lpapiektis ktkgqprepq vytlppsree mtknqvsltc lvkgfypsdi
401 avewesngqp ennykttppm ldsdgsffly skltvdksrw qqgnvfscsv
451 mhealhnhyt qkslslspgk;
(SEQ ID NO:31)
    1 mefglswlfl vailkgvqce vqllesgggl vqpggslrls caas
  51 wvrqap gkglewvsrftis rdnskntlvl
101 qmnslraedt avyycakwgqgttv tvssastkgp
151 svfplapcsr stsestaalg clvkdyfpep vtvswnsgal tsgvhtfpav
201 lqssglysls svvtvpssnf gtqtytcnvd hkpsntkvdk tverkccvec
251 ppcpappvag psvflfppkp kdtlmisrtp evtcvvvdvs hedpevqfnw
301 yvdgvevhna ktkpreeqfn stfrvvsvlt vvhqdwlngk eykckvsnkg
351 lpapiektis ktkgqprepq vytlppsree mtknqvsltc lvkgfypsdi
401 avewesngqp ennykttppm ldsdgsffly skltvdksrw qqgnvfscsv
451 mhealhnhyt qkslslspgk;
or
(SEQ ID NO:32)
    1 mefglswlfl vailkgvqce vqllesgggl vqpggslrls ctas
  51 wvrqap gkglewvsrftis rdnsrttlyl
101 qmnslraedt avyycakwgqgttv tvssastkgp
151 svfplapcsr stsestaalg clvkdyfpep vtvswnsgal tsgvhtfpav
201 lqssglysls svvtvpssnf gtqtytcnvd hkpsntkvdk tverkccvec
251 ppcpappvag psvflfppkp kdtlmisrtp evtcvvvdvs hedpevqfnw
301 yvdgvevhna ktkpreeqfn stfrvvsvlt vvhqdwlngk eykckvsnkg
351 lpapiektis ktkgqprepq vytlppsree mtknqvsltc lvkgfypsdi
401 avewesngqp ennykttppm ldsdgsffly skltvdksrw qqgnvfscsv
451 mhealhnhyt qkslslspgk.
```

In an embodiment of the invention, the signal sequence is amino acids 1-19 of SEQ ID NOs: 29-32. In an embodiment of the invention, the mature variable region is underscored. In an embodiment of the invention, the anti-IGF1R antibody or antigen-binding fragment thereof of the invention comprises one or more CDRs (e.g., 3 light chain CDRS) as set forth above.

In an embodiment of the invention, the anti-IGF1R antibody comprises a light chain variable region comprising the amino acid sequence of any of SEQ ID NOs: 19-24 paired with a heavy chain variable region comprising an amino acid sequence of any of SEQ ID NOs: 13-18, respectively. In an embodiment of the invention, the anti-IGF1R antibody comprises a mature light chain variable region comprising an amino acid sequence of any of SEQ ID NOs: 25 or 26 paired with a heavy chain variable region comprising an amino acid sequence of any of SEQ ID NOs: 29 or 30. In an embodiment of the invention, the anti-IGF1R antibody comprises a mature light chain variable region comprising an amino acid sequence of any of SEQ ID NOs: 27 or 28 paired with a heavy chain variable region comprising an amino acid sequence of any of SEQ ID NOs: 31 or 32.

In an embodiment of the invention, an anti-IGF1R antibody or antigen-binding fragment thereof of the invention comprises an immunoglobulin heavy chain or mature fragment or variable region of 2.12.1 fx (SEQ ID NO: 33) (in an embodiment of the invention, the leader sequence is underscored; in an embodiment of the invention, the CDRs are in bold/italicized font):

```
    1 mefglswvfl vaiikgvqcq vqlvesgggl vkpggslrls caas [symbol]
  51 [symbol] wirqap gkglewvs [symbol] rftis rdnaknslyl
101 qmnslraedt avyycar [symbol] wgqgttv tvssastkgp
151 svfplapcsr stsestaalg clvkdyfpep vtvswnsgal tsgvhtfpav
201 lqssglysls svvtvpssnf gtqtytcnvd hkpsntkvdk tverkccvec
251 ppcpappvag psvflfppkp kdtlmisrtp evtcvvvdvs hedpevqfnw
301 yvdgvevhna ktkpreeqfn stfrvvsvlt vvhqdwlngk eykckvsnkg
351 lpapiektis ktkgqprepq vytlppsree mtknqvsltc lvkgfypsdi
401 avewesngqp ennykttppm ldsdgsffly skltvdksrw qqgnvfscsv
451 mhealhnhyt qkslslspgk
```

In an embodiment of the invention, the anti-IGF1R antibody or antigen-binding fragment thereof of the invention comprises amino acids 20-470 of 2.12.1 fx (SEQ ID NO: 33).

In an embodiment of the invention, an anti-IGF1R antibody or antigen-binding fragment thereof of the invention comprises mature immunoglobulin heavy chain variable region 2.12.1 fx (amino acids 20-144 or SEQ ID NO: 33; SEQ ID NO: 34):

q vqlvesgggl vkpggslrls caasgttfsd yymswirqap
gkglewvsyi sssgstrdya dsvkgrftis rdnaknslyl
qmnslraedt avyycardgv ettfyyyyyg mdvwgqgttv
tvss

In an embodiment of the invention, an anti-IGF1R antibody or antigen-binding fragment thereof of the invention comprises an immunoglobulin light chain or mature fragment or variable region 2.12.1 fx (SEQ ID NO: 35) (in an embodiment of the invention, the leader sequence is underscored; in an embodiment of the invention, the CDRs are in bold/italicized font):

```
    1 mdmrvpaqll gllllwfpga rcdiqmtqsp sslsasvgdr vtitc [symbol]
  51 [symbol] wyqq kpgkapkrli y [symbol] gv psrfsgsgsg teftltissl
101 qpedfatyyc [symbol] f gqgtkveikr tvaapsvfif ppsdeqlksg
151 tasvvcllnn fypreakvqw kvdnalqsgn sqesvteqds kdstyslsst
201 ltlskadyek hkvyacevth qglsspvtks fnrgec
```

In an embodiment of the invention, an anti-IGF1R antibody or antigen-binding fragment thereof of the invention comprises amino acids 23-236 of 2.12-1 fx (SEQ ID NO: 35).

In an embodiment of the invention, an anti-IGF1R antibody or antigen-binding fragment thereof of the invention comprises mature immunoglobulin light chain variable region 2.12.1 fx (amino acids 23-130 of SEQ ID NO: 35; SEQ ID NO: 36):

diqmtqsp sslsasvgdr vtitcrasqd irrdlgwyqq
kpgkapkrli yaasrlqsgv psrfsgsgsg teftltissl
qpedfatyyc lqhnnyprtf gqgtkveikr

In an embodiment of the invention, an anti-IGF1R antibody or antigen-binding fragment thereof comprises or consists of a light chain immunoglobulin chain comprising or consisting of amino acids 23-236 of 2.12.1 fx (SEQ ID NO: 35) and a heavy chain immunoglobulin chain comprising or consisting of amino acids 20-470 of 2.12.1 fx (SEQ ID NO: 33).

In an embodiment of the invention, the anti-IGF1R antibody or antigen-binding fragment thereof comprises one or more 2.12.1 fx CDRs (e.g., 3 light chain CDRs and/or 3 heavy chain CDRs) as set forth above.

In an embodiment of the invention, an anti-IGF1R antibody or antigen-binding fragment thereof of the invention or antigen-binding fragment thereof comprises a humanized 7C10 immunoglobulin light chain variable region; version 1 (SEQ ID NO: 37);

```
    1 dvvmtqspls lpvtpgepas iscrssqsiv hsngntylqw ylqkpgqspq
  51 lliykvsnrl ygvpdrfsgs gsgtdftlki srveaedvgv yycfqgshvp
101 wtfgqgtkve ik
```

In an embodiment of the invention, an anti-IGF1R antibody or antigen-binding fragment thereof of the invention comprises humanized 7C10 immunoglobulin light chain variable region, version 2 (SEQ ID NO: 38):

```
    1 divmtqspls lpvtpgepas iscrssqsiv hsngntylqw ylqkpgqspq
  51 lliykvsnrl ygvpdrfsgs gsgtdftlki srveaedvgv yycfqgshvp
101 wtfgqgtkve ik
```

In an embodiment of the invention, an anti-IGF1R antibody or antigen-binding fragment thereof of the invention comprises a humanized 7C10 immunoglobulin heavy chain variable region; version 1 (SEQ ID NO: 39):

```
    1 qyqlqesgpg lvkpsetlsl tctvsgysit gqylwnwirq ppgkglewmg
  51 yisydgtnny kpslkdriti srdtsknqfs lklssvtaad tavyycaryg
101 rvffdywgqg tlvtvss
```

In an embodiment of the invention, an anti-IGF1R antibody or antigen-binding fragment thereof of the invention comprises the humanized 7C10 immunoglobulin heavy chain variable region; version 2 (SEQ ID NO: 40):

```
    1 qvqlqesgpg lvkpsetlsl tctvsgysit ggylwnwirq ppgkglewig
  51 yisydgtnny kpslkdrvti srdtsknqfs lklssvtaad tavyycaryg
101 rvffdywggg tlvtvss
```

In an embodiment of the invention, an anti-IGF1R antibody or antigen-binding fragment thereof of the invention comprises the humanized 7C10 immunoglobulin heavy chain variable region; version 3 (SEQ ID NO: 41):

```
    1 qvqlqesgpg lvkpsetlsl tctvsgysis ggylwnwirq ppgkglewig
  51 yisydgtnny kpslkdrvti svdtsknqfs lklssvtaad tavyycaryg
101 rvffdywgqg tlvtvss
```

In an embodiment of the invention, an anti-IGF1R antibody or antigen-binding fragment thereof of the invention comprises A12 immunoglobulin heavy chain variable region (SEQ ID NO: 42):

```
    1 evqlvqsgae vkkpgssvkv sckasggtfs syaiswvrqa pgqglewmgg
  51 iipifgtany aqkfqgrvti tadkststay melsslrsed tavyycarap
101 lrflewscqd hyyyyymdvw gkgttvtvss
```

In an embodiment of the invention, an anti-IGF1R antibody or antigen-binding fragment thereof of the invention comprises A12 immunoglobulin light chain variable region (SEQ ID NO: 43):

```
    1 sseltqdpav svalgqtvri tcqgdslrsy yaswyqqkpg qapvlviygk
  51 nnrpsgipdr fsqsssgnta sltitgaqae deadyycnsr dnsdnrlifg
101 ggtkltvls
```

or (SEQ ID NO: 106):

```
    1 sseltqdpav svalgqtvri tcqgdslrsy yatwyqqkpg qapilviyge
  51 nkrpsgipdr fsgsssgnta sltitgaqae deadyycksr dgsgqhlvfg
101 ggtkltvlg
```

In an embodiment of the invention, an anti-IGF1R antibody or antigen-binding fragment thereof of the invention comprises 1A immunoglobulin heavy chain variable region (SEQ ID NO: 44):

```
    1 evqlvqsggg lvhpggslrl scagsgftfr nyamywvrqa pgkglewvsa
  51 igsgggtyya dsvkgrftis rdnaknslyl qmnslraedm avyycarapn
101 wgsdafdiwg qgtmvtvss
```

;optionally including one or more of the following mutations: R30, S30, N31, S31, Y94, H94, D104, E104.

In an embodiment of the invention, an anti-IGF1R antibody or antigen-binding fragment thereof of the invention comprises 1A immunoglobulin light chain variable region (SEQ ID NO: 45):

```
    1 diqmtqspss lsasvgdrvt itcrasqgis swlawyqqkp ekapksliya
  51 asslqsgvps rfsgsgsgtd ftltisslqp edfatyycqq ynsypptfgp
101 gtkvdik
```

;optionally including one or more of the following mutations: P96, 196, P100, Q100, R103, K103, V104, L104, D105, E105

In an embodiment of the invention, an anti-IGF1R antibody or antigen-binding fragment thereof of the invention comprises single chain antibody (fv) 8A1 (SEQ ID NO: 46):

```
    1 evqlvqsgae vkkpgeslti sckgpgynff nywigwvrqm pqkglewmgi
  51 iyptdsdtry spsfqgqvti svdksistay lqwsslkasd tamyycarsi
101 rycpggrcys gyygmdvwgq gtmvtvssgg ggsggggsgg ggsseltqdp
151 avsvalgqtv ritcqgdslr syyaswyqqk pgqapvlviy gknnrpsgip
201 drfsgsssgn tasltitgaq aedeadyycn srdssgnhvv fgggtkltvl
251 g
```

In an embodiment of the invention, an anti-IGF1R antibody or antigen-binding fragment thereof of the invention comprises single chain antibody (fv) 9A2 (SEQ ID NO: 47):

```
    1 qvqlvqsgae vrkpgasvkv scktsgytfr nydinwvrqa pgqglewmgr
  51 isghygntdh aqkfqgrftm tkdtststay melrsltfdd tavyycarsq
101 wnvdywgrgt lvtvssgggg sggggsgggg salnfmltqp hsvsespgkt
151 vtisctrssg siasnyvqwy qqrpgssptt vifednrrps gvpdrfsgsi
201 dtssnsaslt isglktedea dyycqsfdst nlvvfgggtk vtvlg
```

In an embodiment of the invention, an anti-IGF1R antibody or antigen-binding fragment thereof of the invention comprises single chain antibody (fv) 11A4 (SEQ ID NO: 48):

```
    1 evqllesggg lvqpggslrl scaasgftfs syamswvrqa pgkglewvsa
  51 isgsggstyy adsvkgrfti srdnskntly lqmnslraed tavyycassp
101 yssrwysfdp wgqgtmvtvs sggggsgggg sggggsalsy eltqppsvsv
151 spgqtatitc sgddlgnkyv swyqqkpgqs pvlviyqdtk rpsgiperfs
201 gsnsgniatl tisgtqavde adyycqvwdt gtvvfgggtk ltvlg
```

In an embodiment of the invention, an anti-IGF1R antibody or antigen-binding fragment thereof of the invention comprises single chain antibody (fv) 7A4 (SEQ ID NO: 49):

```
    1 evqlvqsgae vkkpgeslti sckgsgynff nywigwvrqm pgkdlewmgi
  51 iyptdsdtry spsfqgqvti svdksistay lqwsslkasd tamyycarsi
101 rycpggrcys gyygmdvwgq gtmvtvssgg gssggggsgg ggsseltqdp
151 avsvalgqtv ritcrgdslr nyyaswyqqk pgqapvlviy gknnrpsgip
201 drfsgsssgn tasltitgaq aedeadyycn srdssgnhmv fgggtkltvl
251 g
```

In an embodiment of the invention, an anti-IGF1R antibody or antigen-binding fragment thereof of the invention comprises single chain antibody (fv) 11A1 (SEQ ID NO: 50):

```
    1 evqlvesggg vvqpgrslrl scaasgftfs dfamhwvrqi pgkglewlsg
  51 lrhdgstayy agsvkgrfti srdnsrntvy lqmnslraed tatyycvtgs
101 gssgphafpv wgkgtlvtvs sggggsgggg sggggsalsy vltqppsasg
151 tpgqrvtisc sgsnsnigty tvnwfqqlpg tapklliysn nqrpsgvpdr
201 fsgsksgtsa slaisglqse deadyycaaw ddslngpvfg ggtkvtvlg
```

In an embodiment of the invention, an anti-IGF1R antibody or antigen-binding fragment thereof of the invention comprises single chain antibody (fv) 7A6 (SEQ ID NO: 51)

```
    1 evqlvqsgae vkkpgeslti sckgsgynff nywiqwvrqm pgkglewmgi
  51 iyptdsdtry spstqgqvti svdksistay lqwsslkasd tamyycarsi
101 rycpggrcys gyygmdvwgq gtlvtvssgg ggsggggsgg ggsseltqdp
151 avsvalgqtv ritcqgdslr syytnwfqqk pgqapllvvy aknkrpsgip
201 drfsgsssgn tasltitgaq aedeadyycn srdssgnhvv fgggtkltvl
251 g
```

In an embodiment of the invention, an anti-IGF1R antibody or an antigen-binding fragment thereof (e.g., a heavy chain or light chain immunoglobulin) of the invention comprises one or more complementarity determing regions (CDR) selected from the group consisting of:

| | |
|---|---|
| sywmh; | (SEQ ID NO:52) |
| einpsngrtnynekfkr; | (SEQ ID NO:53) |
| grpdyygsskwyfdv; | (SEQ ID NO:54) |
| rssqsivhsnvntyle; | (SEQ ID NO:55) |
| kvsnrfs; | (SEQ ID NO:56) |
| and | |
| fqgshvppt. | (SEQ ID NO:57) |

In an embodiment of the invention, an anti-IGF1R antibody or an antigen-binding fragment thereof of the invention comprises a heavy chain immunoglobulin variable region selected from the group consisting of:

```
(SEQ ID NO:58)
    1 qvqlvqsgae vvkpgasvkl sckasgytft sywmhwvkqr pgqglewige
  51 inpsngrtny nqkfggkatl tvdkssstay mqlssltsed savyyfargr
101 pdyygsskwy fdvwgqgttv tvs;
(SEQ ID NO:59)
    1 qvqfqqsgae lvkpgasvkl sckasgytft sylmhwikqr pgrglewigr
  51 idpnnvvtkf nekfkskatl tvdkpsstay melssltsed savyycarya
101 ycrpmdywgq gttvtvss;
(SEQ ID NO:60)
    1 qvqlqqsgae lvkpgasvkl sckasgytft sywmhwvkqr pgqglewige
  51 inpsngrtny nekfkrkatl tvdkssstay mqlssltsed savyyfargr
101 pdyygsskwy fdvwgagttv tvs;
(SEQ ID NO:61)
    1 qvqlqqsgae lmkpgasvki sckatgytfs sfwiewvkqr pghglewige
  51 ilpgsggthy nekfkgkatf tadkssntay mqlssltsed savyycargh
101 syyfydgdyw gqgtsvtvss;
(SEQ ID NO:62)
    1 qvqlqqpgsv lvrpgasvkl sckasgytft sswihwakqr pgqglewige
  51 ihpnsgntny nekfkgkatl tvdtssstay vdlssltsed savyycarwr
101 ygspyyfdyw gqgttltvss;
(SEQ ID NO:63)
    1 qvqlqqpgae lvkpgasvkl sckasgytft sywmhwvkqr pgrglewigr
  51 idpnsggtky nekfkskatl tvdkpsstay mqlssltsed savyycaryd
101 yygssyfdyw gqgttltvss;
(SEQ ID NO:64)
    1 qvqlvqsgae vvkpgasvkl sckasgytft sywmhwvkqr pgqglewige
  51 inpsngrtny nqkfqgkatl tvdkssstay mqlssltsed savyyfargr
101 pdyygsskwy fdvwgqgttv tvs;
(SEQ ID NO:65)
    1 qvqlqqsgae lvkpgasvkl sckasgytft sywmhwvkqr pgqglewige
  51 inpsngrtny nekfkrkatl tvdkssstay mqlssltsed savyyfargr
101 pdyygsskwy fdvwgagttv tvss;
(SEQ ID NO:66)
    1 qvqlvqsgae vvkpgasvkl sckasgytft sywmhwvkqr pgqglewige
  51 inpsngrtny nqkfqgkatl tvdkssstay mqlssltsed savyyfargr
101 pdyygsskwy fdvwgqgttv tvss;
(SEQ ID NO:67)
    1 qvqlgqsgae lvkpgasvkl sckasgytft sywmhwvkqr pgrglewigr
  51 idpnsggtky nekfkskatl tvdkpsstay mqlssltsed savyycaryd
101 yygssyfdyw gqgttvtvss;
(SEQ ID NO:68)
    1 qiqlqqsgpe lvrpgasvki sckasgytft dyyihwvkqr pgeglewigw
  51 iypgsgntky nekfkgkatl tvdtssstay mqlssltsed savyfcargg
101 kfamdywgqg tsvtvss;
(SEQ ID NO:69)
    1 qvqlqqsgae lvkpgasvkl sckasgytft sywmhwvkqr pgqglewige
  51 inpsngrtny nekfkrkatl tvdkssstay mqlssltsed savyyfargr
101 pdyygsskwy fdvwgagttv tvss;
(SEQ ID NO:70)
    1 qiqlqqsgpe lvkpgasvki sckasgytft dyyinwmkqk pgqglewigw
  51 idpgsgntky nekfkgkatl tvdtssstay mqlssltsed tavyfcarek
101 ttyyyamdyw gqgtsvtvsa;
(SEQ ID NO:71)
    1 vqlqqsgael mkpgasvkis ckasgytfsd ywiewvkqrp ghglewigei
  51 lpgsgstnyh erfkgkatft adtssstaym qlnsltseds gvyyclhgny
101 dfdgwgqgtt ltvss;
and
(SEQ ID NO:72)
    1 qvqllesgae lmkpgasvki sckatgytfs sfwiewvkqr pghglewige
  51 ilpgsggthy nekfkgkatf tadkssntay mqlssltsed savyycargh
101 syyfydgdyw gqgtsvtvss;
and/or a light chain immunoglobulin variable region selected
from the group consisting of:
(SEQ ID NO:73)
    1 dvlmtqipvs lpvslgdqas iscrssqiiv hnngntylew ylqkpgqspq
  51 lliykvsnrf sgvpdrfsgs gsgtdftlki srveaedlgv yycfqgshvp
101 ftfgsgtkle ikr;
(SEQ ID NO:74)
    1 dvlmtqtpls lpvslgdpas iscrssqsiv hsnvntylew ylqkpgqspk
  51 lliykvsnrf sgvpdrfsgs gagtdftlri srveaedlgi yycfqgshvp
101 ptfgggtkle ikr;
(SEQ ID NO:75)
    1 dvlmtqtpls lpvslgdpas iscrssqsiv hsnvntylew ylqkpgqspr
  51 lliykvsnrf sgvpdrfsgs gagtdftlri srveaedlgi yycfqgshvp
101 ptfgggtkle ikr;
(SEQ ID NO:76)
    1 dvlmtqtpls lpvslgdpas iscrssqsiv hsnvntylew ylqkpgqspk
  51 lliykvsnrf sgvpdrfsgs gagtdftlri srveaedlgi yycfqgshvp
101 ptfgggtkle ikr;
(SEQ ID NO:77)
    1 dvlmtqtpls lpvslgdpas iscrssqsiv hsnvntylew ylqkpgqspr
  51 lliykvsnrf sgvpdrfsgs gagtdftlri srveaedlgi yycfqgshvp
101 ptfgggtkle ikr;
(SEQ ID NO:78)
    1 dvlmtqtpls lpvslgdqas iscrssqxiv hsngntylew ylqkpgqspk
  51 lliykvsnrf sgvpdrfsgs gsgtdftlki srveaedlgv yycfqgshvp
101 xtfgggtkle ikr;
(SEQ ID NO:79)
    1 dvvmtqtpls lpvslgdpas iscrssqsiv hsnvntylew ylqkpgqspk
  51 lliykvsnrf sgvpdrfsgs gagtdftlri srveaedlgi yycfqgshvp
101 ptfgggtkle ikr;
(SEQ ID NO:80)
    1 dvvmtqtpls lpvslgdpas iscrssqsiv hsnvntylew ylqkpgqspr
  51 lliykvsnrf sgvpdrfsgs gagtdftlri srveaedlgi yycfqgshvp
101 ptfgggtkle ikr;
(SEQ ID NO:81)
    1 dvlmtqtpls lpvslgdpas iscrssqsiv hsnvntylew ylqkpgqspr
  51 lliykvsnrf sgvpdrfsgs gagtdftlri srveaedlgi yycfqgshvp
101 ptfgggtkle ikr;
(SEQ ID NO:82)
    1 dvlmtqipvs lpvslgdqas iscrssqiiv hnngntylew ylqkpgqspq
  51 lliykvsnrf sgvpdrfsgs gsgtdftlki srveaedlgv yycfqgshvp
101 ftfgsgtkle ikr;
(SEQ ID NO:83)
    1 dvlmtqtpls lpvslgdqas iscrfsqsiv hsngntylew ylqksgqspk
  51 lliykvsnrf sgvpdrfsgs gsgtdftlki srveaedlgv yycfqgshvp
101 rtfgggtkle ikr;
(SEQ ID NO:84)
    1 dvlmtqtpls lpvslgdqas iscrssqsiv hsnvntylew ylqkpgqspk
  51 lliykvsnrf sgvpdrfsgs gsgtdftlri srveaedlgi yycfqgshvp
101 ptfgggtkle ikr;
(SEQ ID NO:85)
    1 dvvmtqtpls lpvslgdpas iscrssqsiv hsnvntylew ylqkpgqspk
  51 lliykvsnrf sgvpdrfsgs gagtdftlri srveaedlgi yycfqgshvp
101 ptfgggtkle ikr;
(SEQ ID NO:86)
    1 elvmtqtpls lpvslgdqas iscrssqtiv hsngdtyldw flqkpgqspk
  51 lliykvsnrf sgvpdrfsgs gsgtdftlki srveaedlgv yycfqgshvp
101 ptfgggtkle ikr;
(SEQ ID NO:87)
    1 dvlmtqtpls lpvslgdpas iscrssqsiv hsnvntylew ylqkpgqspk
  51 lliykvsnrf sgvpdrfsgs gagtdftlri srveaedlgi yycfqgshvp
101 ptfgggtkle ikr;
(SEQ ID NO:88)
    1 dvvmtqtpls lpvslgdpas iscrssqsiv hsnvntylew ylqkpgqspr
  51 lliykvsnrf sgvpdrfsgs gagtdftlri srveaedlgi yycfqgshvp
101 ptfgggtkle ikr;
(SEQ ID NO:89)
    1 dvlmtqtpvs lsvslgdqas iscrssgsiv hstgntylew ylqkpgqspk
  51 lliykisnrf sgvpdrfsgs gsgtdftlki srveaedlgv yycfqashap
101 rtfgggtkle ikr;
(SEQ ID NO:90)
    1 dvlmtqtpls lpvslgdqas isckssqsiv hssgntyfew ylqkpgqspk
  51 lliykvsnrf sgvpdrtsgs gsgtdftlki srveaedlgv yycfqgship
101 ftfgsgtkle ikr;
(SEQ ID NO:91)
    1 dieltqtpls lpvslgdqas iscrssqsiv hsngntylew ylqkpgqspk
  51 lliykvsnrf sgvpdrfsgs gsgtdftlki srveaedlgv yycfqgshvp
101 ytfgggtkle ikr;
(SEQ ID NO:92)
    1 dvlmtqtpls lpvslgdqas iscrssqsiv hsnvntylew ylqkpgqspk
  51 lliykvsnrf sgvpdrfsgs gsgtdftlri srveaedlgi yycfqgshvp
101 ptfgqgtkle ikr;
(SEQ ID NO:93)
    1 dvvmtqtpls lpvslgdpas iscrssqsiv hsnvntylew ylqkpgqspr
  51 lliykvsnrf sgvpdrfsgs gagtdftlri srveaedlgi yycfqgshvp
101 ptfgggtkle ikr;
(SEQ ID NO:94)
    1 dvlmtqtpls lpvslgdqas iscrssqsiv hsnvntylew ylqkpgqspk
  51 lliykvsnrf sgvpdrfsgs gsgtdftlri srveaedlgi yycfqgshvp
101 ptfgggtkle ikr;
(SEQ ID NO:95)
    1 dvvmtqtpls lpvslgdpas iscrssqsiv hsnvntylew ylqkpgqspk
  51 lliykvsnrf sgvpdrfsgs gagtdftlri srveaedlgi yycfqgshvp
101 ptfgggtkle ikr;
(SEQ ID NO:96)
    1 dvlmtqtpls lpvslgdqas iscrsnqtil lsdgdtylew ylqkpgqspk
  51 lliykvsnrf sgvpdrfsgs gsgtdftlki srveaedlgv yycfqgshvp
101 ptfgggtkle ikr;
(SEQ ID NO:97)
    1 dvlmtqtpls lpvslgdqas iscrssqtiv hsngntylew ylqkpgqspk
  51 lliykvtnrf sgvpdrfsgs gsgtdftlki srveaedlgv yycfqgthap
101 ytfgggtkle ikr;
and
(SEQ ID NO:98)
    1 dvlmtqtpls lpvslgdqas iscrssqsiv hsngntylew ylqkpgqspk
  51 lliysissrf sgvpdrfsgs gsgtdftlki srvqaedlgv yycfqgshvp
101 ytfgggtkle ikr.
```

The scope of the present invention includes methods wherein a patient is administered an anti-insulin-like growth factor receptor-1 (IGF1R) antibody wherein the variable region of the antibody is linked to any immunoglobulin constant region. In an embodiment, the light chain variable region is linked to a κ chain constant region. In an embodiment, the heavy chain variable region is linked to a γ1, γ2, γ3 or γ4 chain constant region. Any of the immunoglobulin variable regions set forth herein, in embodiments of the invention, can be linked to any of the foregoing constant regions.

Furthermore, the scope of the present invention comprises any antibody or antibody fragment comprising one or more CDRs (3 light chain CDRs and/or 3 heavy chain CDRs) and/or framework regions of any of the light chain immunoglobulin or heavy chain immunoglobulins set forth herein as identified by any of the methods set forth in Chothia et al., J. Mol. Biol. 186:651-663 (1985); Novotny and Haber, Proc. Natl. Acad. Sci. USA 82:4592-4596 (1985) or Kabat, E. A. et al., Sequences of Proteins of Immunological Interest, National Institutes of Health, Bethesda, Md., (1987)).

In an embodiment of the invention, the term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modifier “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, et al., (1975) Nature 256; 495.

In an embodiment of the invention, a polyclonal antibody is an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

In an embodiment of the invention, a bispecific or bifunctional antibody is an artificial hybrid antibody having two different heavy/light chain pairs and two different binding sites. Bispecific antibodies can be produced by a variety of methods including fusion of hybridomas or linking of Fab' fragments. See, e.g., Songsivilai, et al., (1990) Clin. Exp. Immunol. 79: 315-321, Kostelny, et al., (1992) J Immunol. 148:1547-1553. In addition, bispecific antibodies may be formed as “diabodies” (Holliger, et al., (1993) Proc. Nat. Acad. Sci. USA 90:6444-6448) or as “Janusins” (Traunecker, et al., (1991) EMBO J. 10:3655-3659 and Traunecker, et al., (1992) Int. J. Cancer Suppl. 7:51-52).

In an embodiment of the invention, the term “fully human antibody” refers to an antibody which comprises human immunoglobulin protein sequences only. A fully human antibody may contain murine carbohydrate chains if produced in a mouse, in a mouse cell or in a hybridoma derived from a mouse cell. Similarly, “mouse antibody” refers to an antibody which comprises mouse immunoglobulin protein sequences only.

The present invention includes “chimeric antibodies”—in an embodiment of the invention, an antibody which comprises a variable region of the present invention fused or chimerized with an antibody region (e.g., constant region) from another, human or non-human species (e.g., mouse, horse, rabbit, dog, cow, chicken). These antibodies may be used to modulate the expression or activity of IGF1R in a non-human species.

“Single-chain Fv” or “sFv” antibody fragments have, in an embodiment of the invention, the V_{H} and V_{L} domains of an antibody, wherein these domains are present in a single polypeptide chain. Generally, the sFv polypeptide further comprises a polypeptide linker between the V_{H} and V_{L} domains which enables the sFv to form the desired structure for antigen binding. Techniques described for the production of single chain antibodies (U.S. Pat. Nos. 5,476,786; 5,132,405 and 4,946,778) can be adapted to produce anti-IGF1R-specific single chain antibodies. For a review of sFv see Pluckthun in The Pharmacology of Monoclonal Antibodies, vol. 113, Rosenburg and Moore eds. Springer-Verlag, N.Y., pp. 269-315 (1994).

In an embodiment of the invention, “disulfide stabilized Fv fragments” and “dsFv” refer to immunoglobulins comprising a variable heavy chain (V_{H}) and a variable light chain (V_{L}) which are linked by a disulfide bridge.

Antigen-binding fragments of antibodies within the scope of the present invention also include F(ab)₂ fragments which may, in an embodiment of the invention, be produced by enzymatic cleavage of an IgG by, for example, pepsin. Fab fragments may be produced by, for example, reduction of F(ab)₂ with dithiothreitol or mercaptoethylamine. A Fab fragment is, in an embodiment of the invention, a V_{L}—C_{L} chain appended to a V_{H}—C_{H1} chain by a disulfide bridge. A F(ab)₂ fragment is, in an embodiment of the invention, two Fab fragments which, in turn, are appended by two disulfide bridges. The Fab portion of an F(ab)₂ molecule includes, in an embodiment of the invention, a portion of the F_{c} region between which disulfide bridges are located.

In an embodiment of the invention, an F_{V} fragment is a V_{L} or V_{H} region.

Depending on the amino acid sequences of the constant domain of their heavy chains, immunoglobulins can be assigned to different classes. There are at least five major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. As discussed herein, any such antibody or antigen-binding fragment thereof is within the scope of the present invention.

The anti-IGF1R antibodies of the invention may, in an embodiment of the invention, be conjugated to a chemical moiety. The chemical moiety may be, inter alia, a polymer, a radionuclide or a cytotoxic factor. In an embodiment of the invention, the chemical moiety is a polymer which increases the half-life of the antibody or antigen-binding fragment thereof in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments of the invention may, in an embodiment of the invention, be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe.

The antibodies and antibody fragments of the invention may also be, in an embodiment of the invention, conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibodies and antibody fragments may also be, in an embodiment of the invention, conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibodies or antigen-binding fragments thereof of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

In an embodiment of the invention, an IGF1R inhibitor is BMS-577098

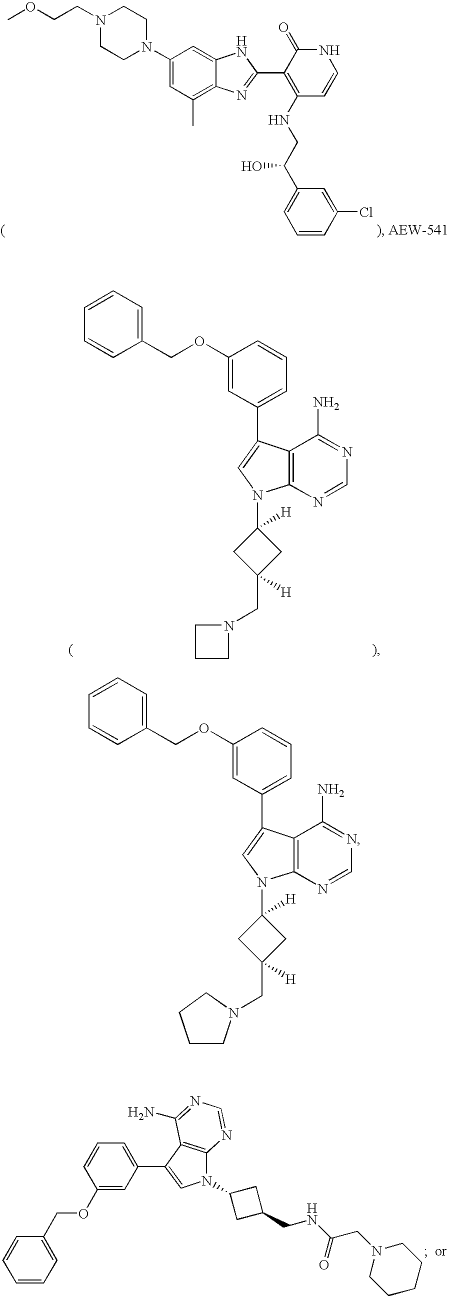

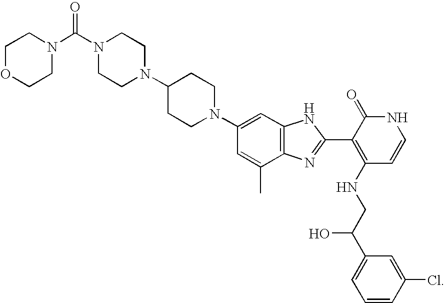

### Generation of Antibodies

Any suitable method can be used to elicit an antibody with the desired biologic properties to inhibit IGF1R. It may be desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES. PRINCIPLES AND PRACTICE (2d ed.) Academic Press, New York, N.Y. Thus, monoclonal antibodies may be obtained by a variety of techniques familiar to researchers skilled in the art. Typically, spleen cells from an animal immunized with a desired antigen are immortalized, commonly by fusion with a myeloma cell. See Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Also, recombinant immunoglobulins may be produced, see Cabilly U.S. Pat. No. 4,816,567; and Queen et al. (1989) Proc. Nat'l Acad. Sci. USA 86:10029-10033; or made in transgenic mice, see Mendez et al. (1997) Nature Genetics 15:146-156. Further methods for producing chimeric, humanized and human antibodies are well known in the art. See, e.g., U.S. Pat. No. 5,530,101, issued to Queen et al, U.S. Pat. No. 5,225,539, issued to Winter et al, U.S. Pat. No. 4,816,397 issued to Boss et al, all of which are incorporated by reference in their entirety.

Mammalian cell lines available as hosts for expression of antibodies of the invention are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen-binding portion thereof, the light chain and/or antigen-binding portion thereof are introduced into mammalian host cells, the antibodies are produced by culturing the host cells for a period of time sufficient to allow for expression of the antibody in the host cells or, in an embodiment of the invention, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in connection with European Patent Nos. 0 216 846, 0 256 055, and 0 323 997 and European Patent Application No. 89303964.4.

It is likely that antibodies expressed by different cell lines or in transgenic animals will have different glycosylation from each other. However, all antibodies encoded by the nucleic acid molecules provided herein, or comprising the amino acid sequences provided herein are part of the instant invention, regardless of the glycosylation of the antibodies.

A convenient plasmid system useful for producing an anti-IGF1R antibody or antigen-binding fragment thereof is set forth in published U.S. application no US2005/0176099 (see also WO2005/47512).

### Further Chemotherapeutics

The scope of the present invention comprises methods for treating a tumor which expresses IGF1R by administering an IGF1R inhibitor, e.g., as discussed herein, in association with a further chemotherapeutic agent or procedure. A further chemotherapeutic agent comprises any agent that elicits a beneficial physiological response in an individual to which it is administered; for example, wherein the agent alleviates or eliminates disease symptoms or causes within the subject to which it is administered. A further chemotherapeutic agent includes any anti-cancer chemotherapeutic agent. An anti-cancer therapeutic agent is any agent that, for example, alleviates or eliminates symptoms or causes of cancer in the subject to which it is administered.

In an embodiment of the invention, the further chemotherapeutic agent is one or more of etoposide (VP-16; 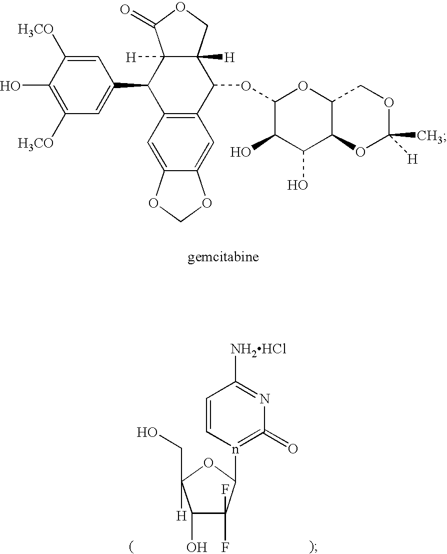
any compound disclosed in published U.S. patent application no. U.S. 2004/0209878A1 (e.g., comprising a core structure represented by 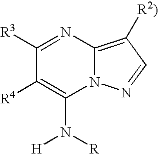
or doxorubicin 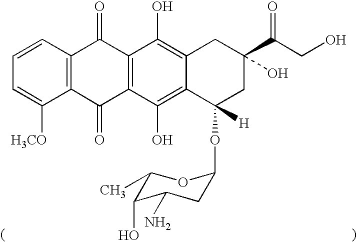
including Caelyx or Doxil® (doxorubicin HCl liposome injection; Ortho Biotech Products L.P; Raritan, N.J.). Doxil® comprises doxorubicin in STEALTH® liposome carriers which are composed of N-(carbonyl-methoxypolyethylene glycol 2000)-1,2-distearoyl-sn-glycero-3-phosphoethanolamine sodium salt (MPEG-DSPE); fully hydrogenated soy phosphatidylcholine (HSPC), and cholesterol.

In an embodiment of the invention, the further chemotherapeutic agent is one or more of 5′-deoxy-5-fluorouridine 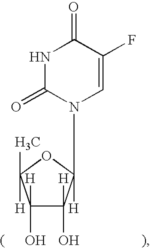
vincristine 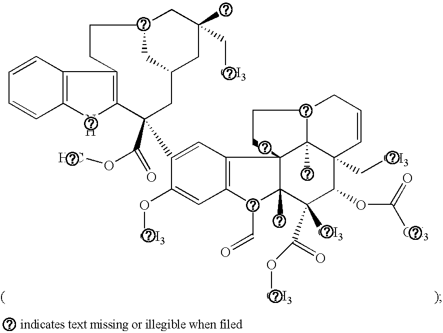
or temozolomide

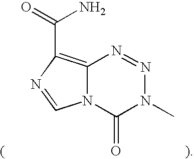

In an embodiment of the invention, the further chemotherapeutic agent is one or more of any CDK inhibitor such as ZK-304709, Seliciclib (R-roscovitine) 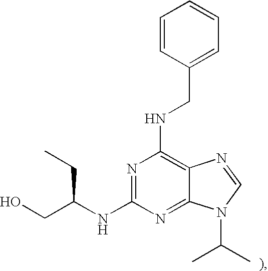
or any MEK inhibitor such as PD0325901 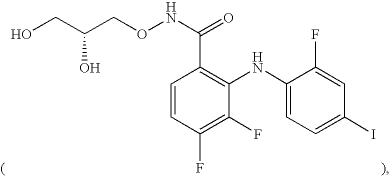
AZD-6244; capecitabine (5′-deoxy-5-fluoro-N-[(pentyloxy)carbonyl]-cytidine); or L-Glutamic acid, N-[4-[2-(2-amino-4,7-dihydro-4-oxo-1H -pyrrolo[2,3-d]pyrimidin-5-yl)ethyl]benzoyl]-, disodium salt, heptahydrate 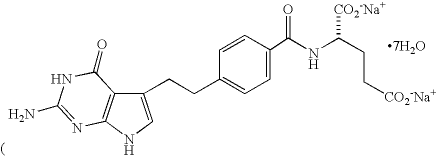
;Pemetrexed disodium heptahydrate).

In an embodiment of the invention, the further chemotherapeutic agent is one or more of camptothecin 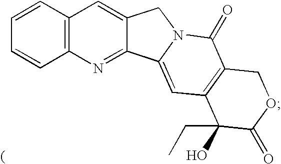
Stork et al., J. Am. Chem. Soc. 93(16): 4074-4075 (1971); Beisler et al., J. Med. Chem. 14(11): 1116-1117 (1962)); or irinotecan 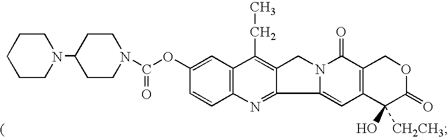
sold as Camptosar®; Pharmacia & Upjohn Co.; Kalamazoo, Mich.).

In an embodiment of the invention, the further chemotherapeutic agent is the FOLFOX regimen (oxaliplatin 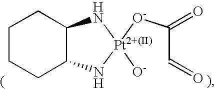
together with infusional fluorouracil 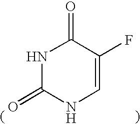
and folinic acid 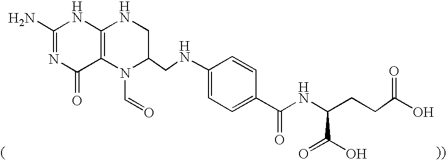
(Chaouche et al., Am. J. Clin. Oncol. 23(3):288-289 (2000); de Gramont et al., J. Clin. Oncol. 18(16):2938-2947 (2000)).

In an embodiment of the invention, the further chemotherapeutic agent is one or more of any antiestrogen such as 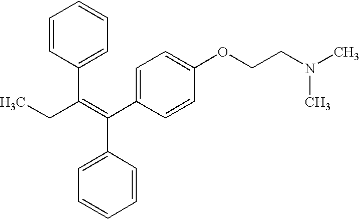
(tamoxifen; sold as Nolvadex® by AstraZeneca Pharmaceuticals LP; Wilmington, Del.) or 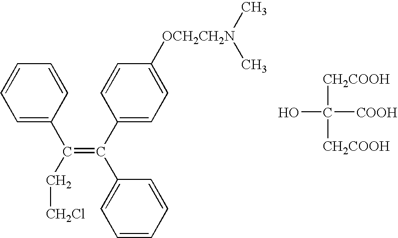
toremifene citrate sold as Fareston® by Shire US, Inc.; Florence, Ky.).

In an embodiment of the invention, the further chemotherapeutic agent is one or more of any aromatase inhibitor such as 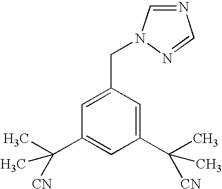
(anastrazole; sold as Arimidex® by AstraZeneca Pharmaceuticals LP; Wilmington, Del.), 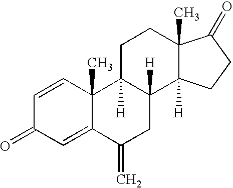
(exemestane; sold as Aromasin® by Pharmacia Corporation; Kalamazoo, Mich.) or 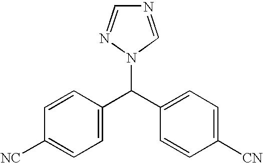
(letrozole; sold as Femara® by Novartis Pharmaceuticals Corporation; East Hanover, N.J.).

In an embodiment of the invention, the further chemotherapeutic agent is one or more of any estrogen such as DES(diethylstilbestrol), 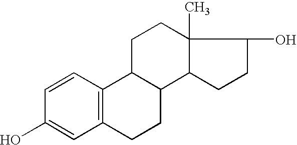
(estradiol; sold as Estrol® by Warner Chilcott, Inc.; Rockaway, N.J.) or conjugated estrogens (sold as Premarin® by Wyeth Pharmaceuticals Inc.; Philadelphia, Pa.).

In an embodiment of the invention, the further chemotherapeutic agent is one or more of any anti-angiogenesis agent including bevacizumab (Avastin™; Genentech; San Francisco, Calif.), the anti-VEGFR-2 antibody IMC-1C11, other VEGFR inhibitors such as: CHIR-258 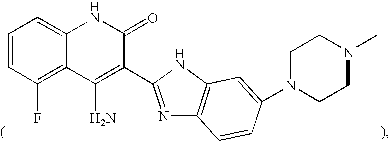
any of the inhibitors set forth in WO2004/13145 (e.g., comprising the core structural formula: 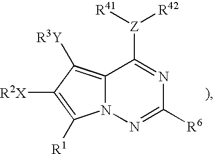
in WO2004/09542 (e.g., comprising the core structural formula: 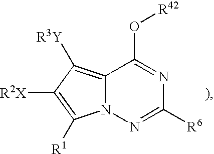
in WO 00/71129 (e.g., comprising the core structural formula: 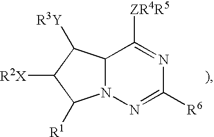
in WO2004/09601 (e.g., comprising the core structural formula: 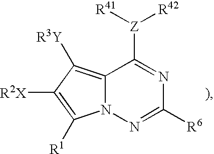
in WO2004/01059 (e.g., comprising the core structural formula: 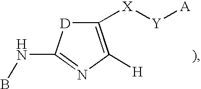
in WO01/29025 (e.g., comprising the core structural formula: 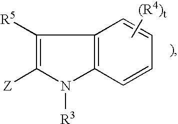
in WO02/32861 (e.g., comprising the core structural formula: 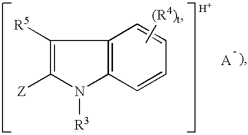
or set forth in WO03/88900 (e.g., comprising the core structural formula 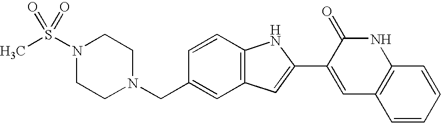
3-[5-(methylsulfonylpiperadinemethyl)-indolyl]-quinolone; Vatalanib 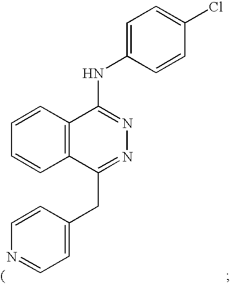
PTK/ZK; CPG-79787; ZK-222584), AG-013736 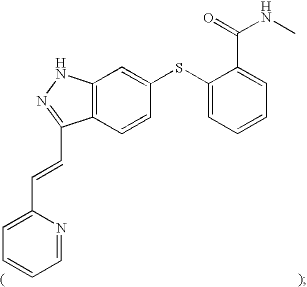
or the VEGF trap (AVE-0005), a soluble decoy receptor comprising portions of VEGF receptors 1 and 2.

In an embodiment of the invention, the further chemotherapeutic agent is one or more of any LHRH (Lutenizing hormone-releasing hormone) agonist such as the acetate salt of [D-Ser(Bu t) 6, Azgly 10](pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4]; 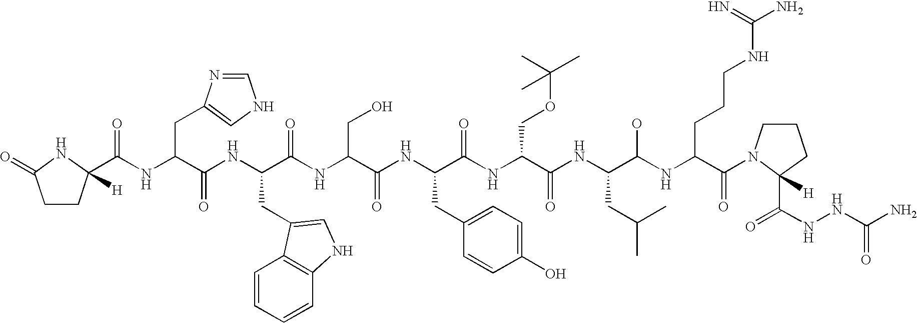
(goserelin acetate; sold as Zoladex® by AstraZeneca UK Limited; Macclesfield, England), 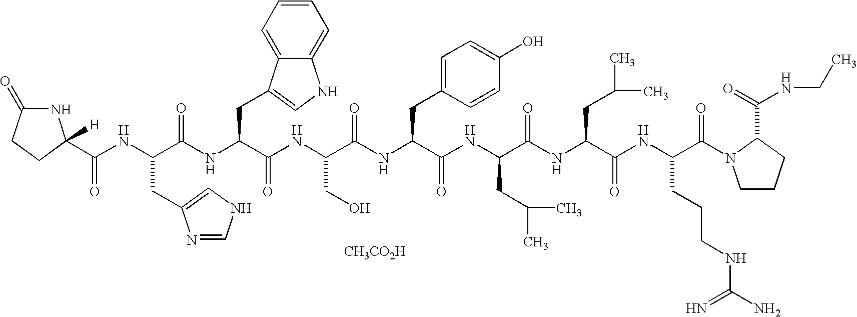
(leuprolide acetate; sold as Eligard® by Sanofi-Synthelabo Inc.; New York N.Y.) or 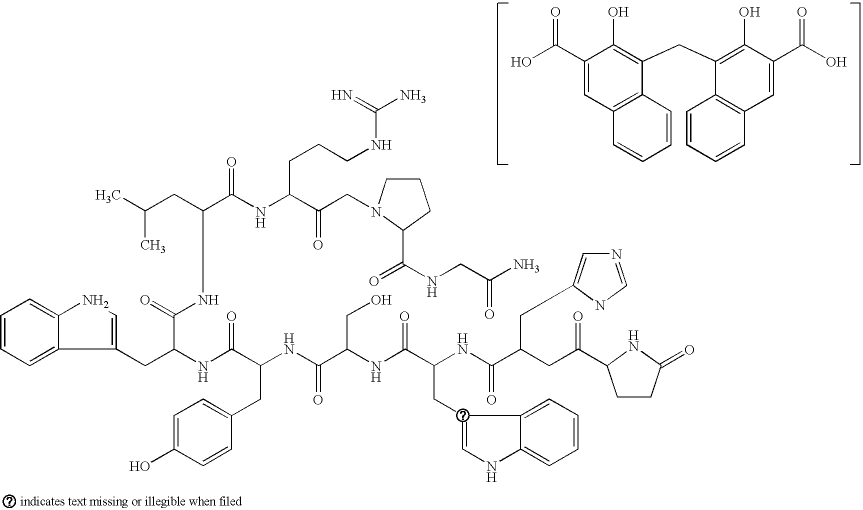
(triptorelin pamoate; sold as Trelstar® by Pharmacia Company, Kalamazoo, Mich.).

In an embodiment of the invention, the further chemotherapeutic agent is one or more of any progestational agent such as 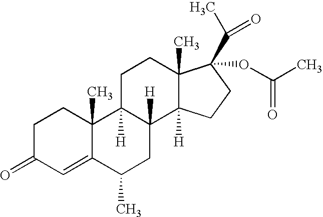
(medroxyprogesterone acetate; sold as Provera® by Pharmacia & Upjohn Co.; Kalamazoo, Mich.), 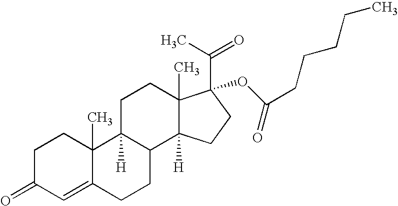
(hydroxyprogesterone caproate; 17-((1-Oxohexyl)oxy)pregn-4-ene-3,20-dione;), megestrol acetate or progestins.

In an embodiment of the invention, the further chemotherapeutic agent is one or more selective estrogen receptor modulators (SERM) such as 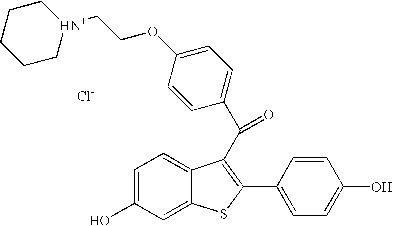
(raloxifene; sold as Evista® by Eli Lilly and Company; Indianapolis, Ind.).

In an embodiment of the invention, the further chemotherapeutic agent is one or more of any anti-androgen including, but not limited to: 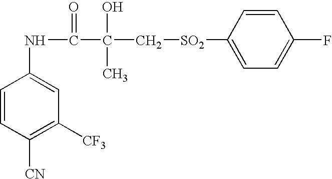
(bicalutamide; sold at CASODEX® by AstraZeneca Pharmaceuticals LP; Wilmington, Del.); 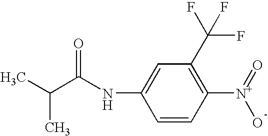
(flutamide; 2-methyl-N-[4-nitro-3(trifluoromethyl)phenyl]propanamide; sold as Eulexin® by Schering Corporation; Kenilworth, N.J.); 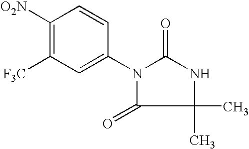
(nilutamide; sold as Nilandron® by Aventis Pharmaceuticals Inc.; Kansas City, Mo.) and 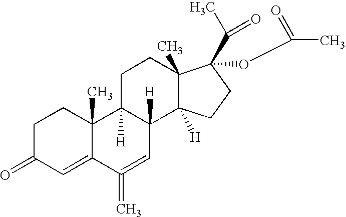
(Megestrol acetate; sold as Megace® by Bristol-Myers Squibb).

In an embodiment of the invention, the further chemotherapeutic agent is one or more of any EGF Receptor or HER2 antagonist, including, but not limited to, CP-724714 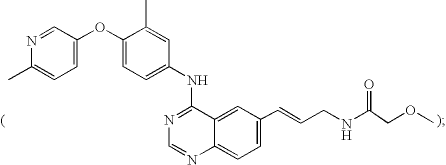
TAK-165 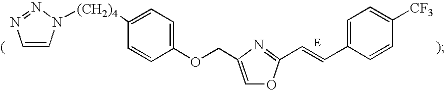
HKI-272 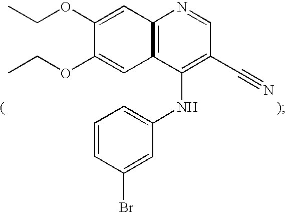
OSI-774 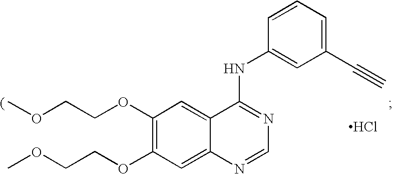
.HCl; erlotinib, Hidalgo et al., J. Clin. Oncol. 19(13): 3279-3279 (2001)), Lapatanib 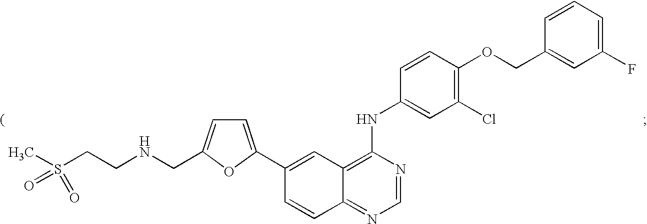
GW2016; Rusnak et al., Molecular Cancer Therapeutics 1:85-94 (2001); N-{3-Chloro-4-[(3-fluorobenzyl)oxy]phenyl}-6-[5-({[2-(methylsulfonyl)ethyl]amino}methyl)-2-furyl]-4-quinazolinamine; PCT Application No. WO99/35146), Canertinib (CI-1033; 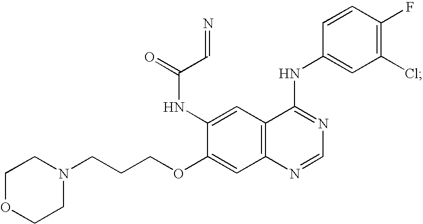
Erlichman et al., Cancer Res. 61(2):739-48 (2001); Smaill et al., J. Med. Chem. 43(7):1380-97 (2000)), ABX-EGF antibody (Abgenix, Inc.; Freemount, Calif.; Yang et al., Cancer Res. 59(6):1236-43 (1999); Yang et al., Crit Rev Oncol Hematol. 38(1):17-23 (2001)), erbitux (U.S. Pat. No. 6,217,866; IMC-C225, cetuximab; Imclone; New York, N.Y.) EKB-569 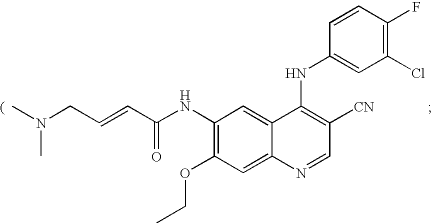
Wissner et al., J. Med. Chem. 46(1): 49-63 (2003)), PKI-166 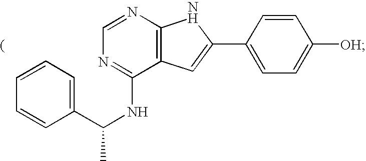
CGP-75166), GW-572016, any anti-EGFR antibody and any anti-HER2 antibody.

In an embodiment of the invention, the further chemotherapeutic agent is one or more of any farnesyl protein transferase inhibitor including 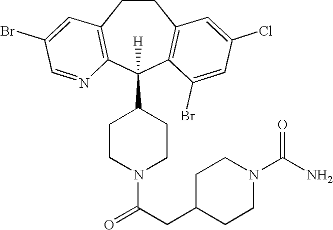
(lonafarnib; Sarasar™; Schering-Plough; Kenilworth, N.J.), 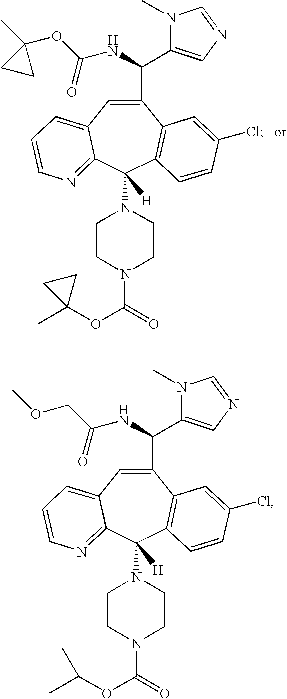
BMS-214662 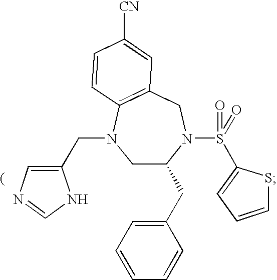
Hunt et al., J. Med. Chem. 43(20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)) and R155777 (tipifarnib; Garner et al., Drug Metab. Dispos. 30(7):823-30 (2002); Dancey et al., Curr. Pharm. Des. 8:2259-2267 (2002); (B)-6-[amino(4-chlorophenyl)(1-methyl-1H-imidazol-5-yl)-methyl]-4-(3-chlorophenyl)-1-methyl-2(1H)-quinolinone]; 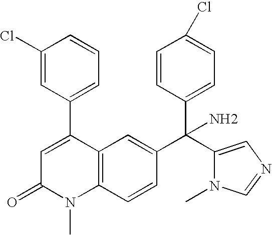
sold as Zarnestra™; Johnson & Johnson; New Brunswick, N.J.).

In an embodiment of the invention, the further chemotherapeutic agent is one or more of any of 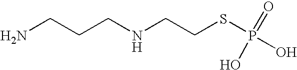
(Amifostine); 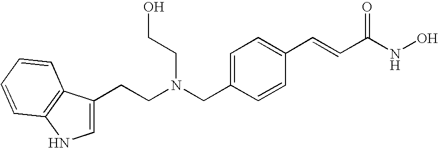
(NVP-LAQ824, Atadja et al., Cancer Research 64: 689-695 (2004)), 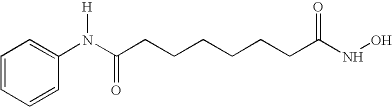
(suberoyl analide hydroxamic acid), 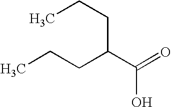
(Valproic acid; Michaelis et al., Mol. Pharmacol. 65:520-527 (2004)), 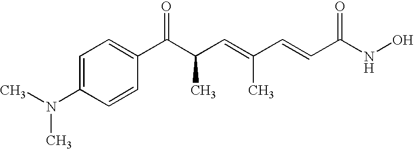
(trichostatin A), 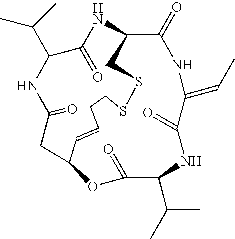
(FK-228; Furumai et al., Cancer Research 62: 4916-4921 (2002)), 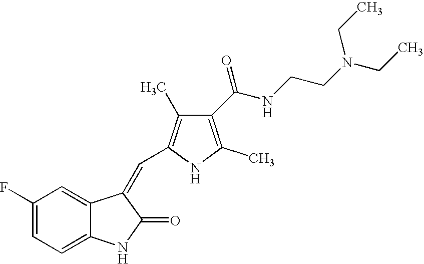
(SU11248; Mendel et al., Clin. Cancer Res. 9(1):327-37 (2003)), 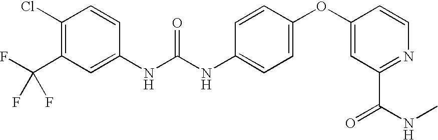
(BAY43-9006), 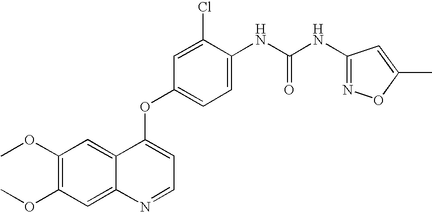
(KRN951), 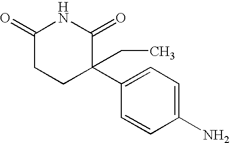
(Aminoglutethimide); 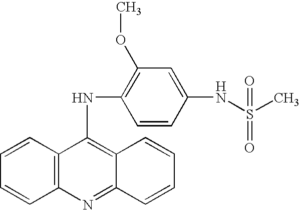
(Amsacrine); 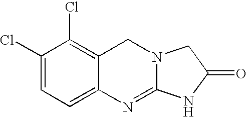
(Anagrelide); 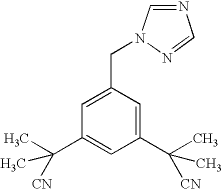
(Anastrozole; sold as Arimidex by AstraZeneca Pharmaceuticals LP; Wilmington, Del.); Asparaginase; Bacillus Calmette-Guerin (BCG) vaccine (Garrido et al., Cytobios. 90(360):47-65 (1997)); 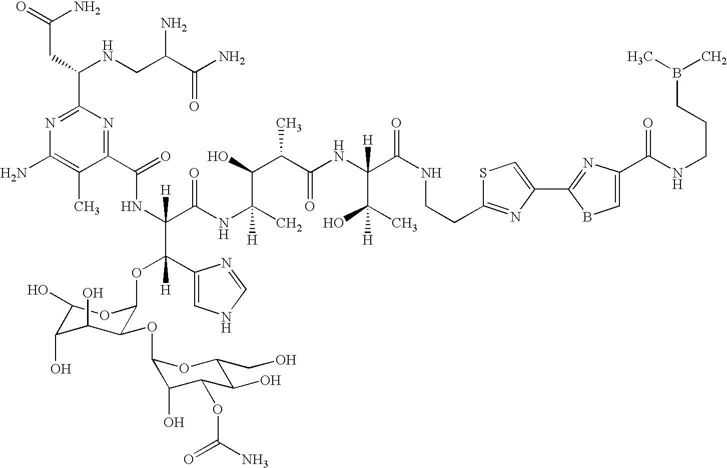
(Bleomycin); 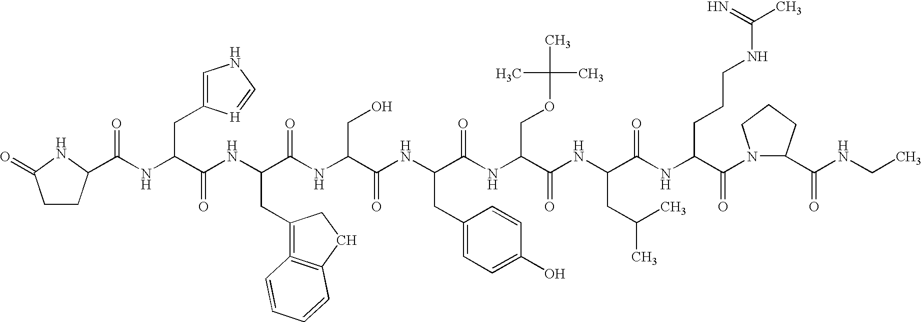
(Buserelin); 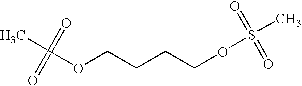
(Busulfan; 1,4-butanediol, dimethanesulfonate; sold as Busulfex® by ESP Pharma, Inc.; Edison, N.J); 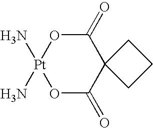
(Carboplatin; sold as Paraplatin® by Bristol-Myers Squibb; Princeton, N.J.); 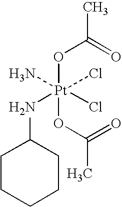
(satraplatin), 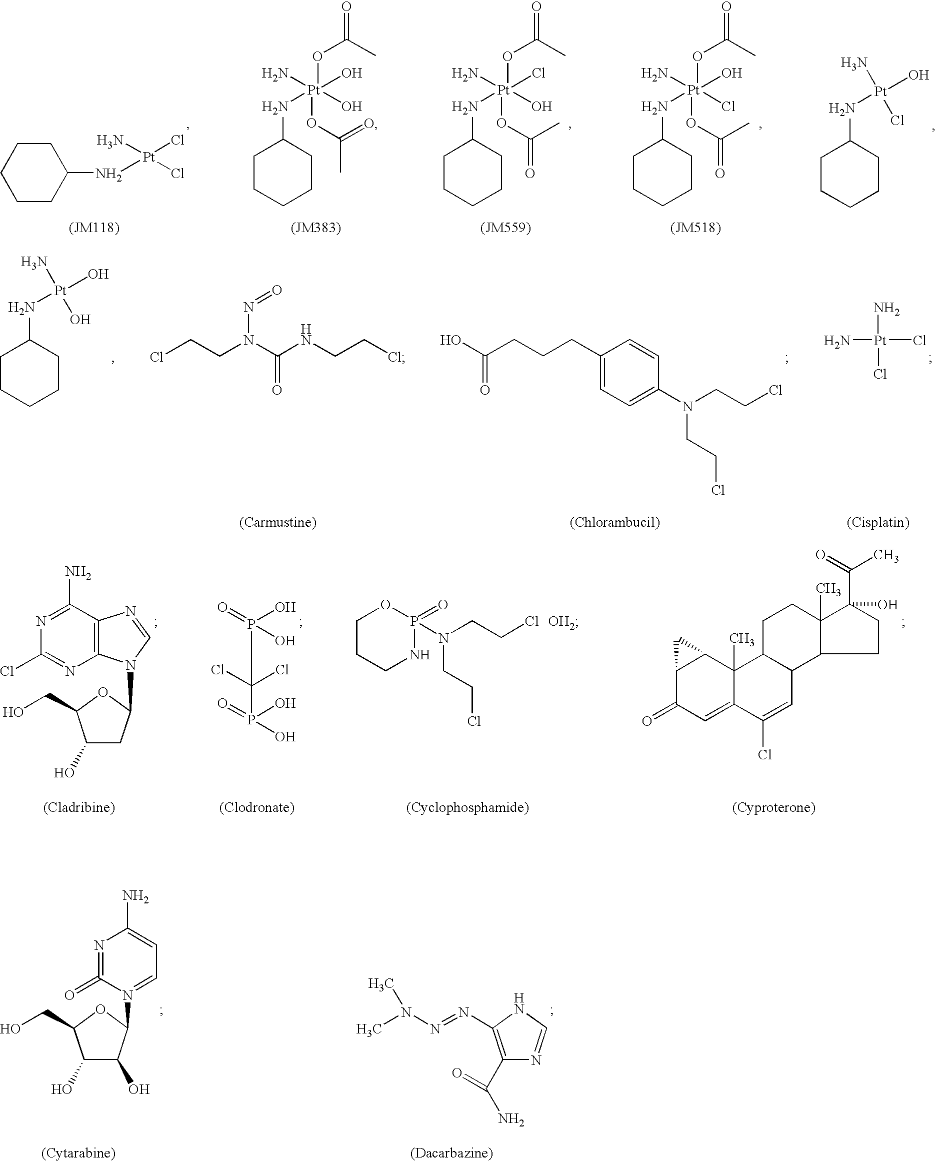 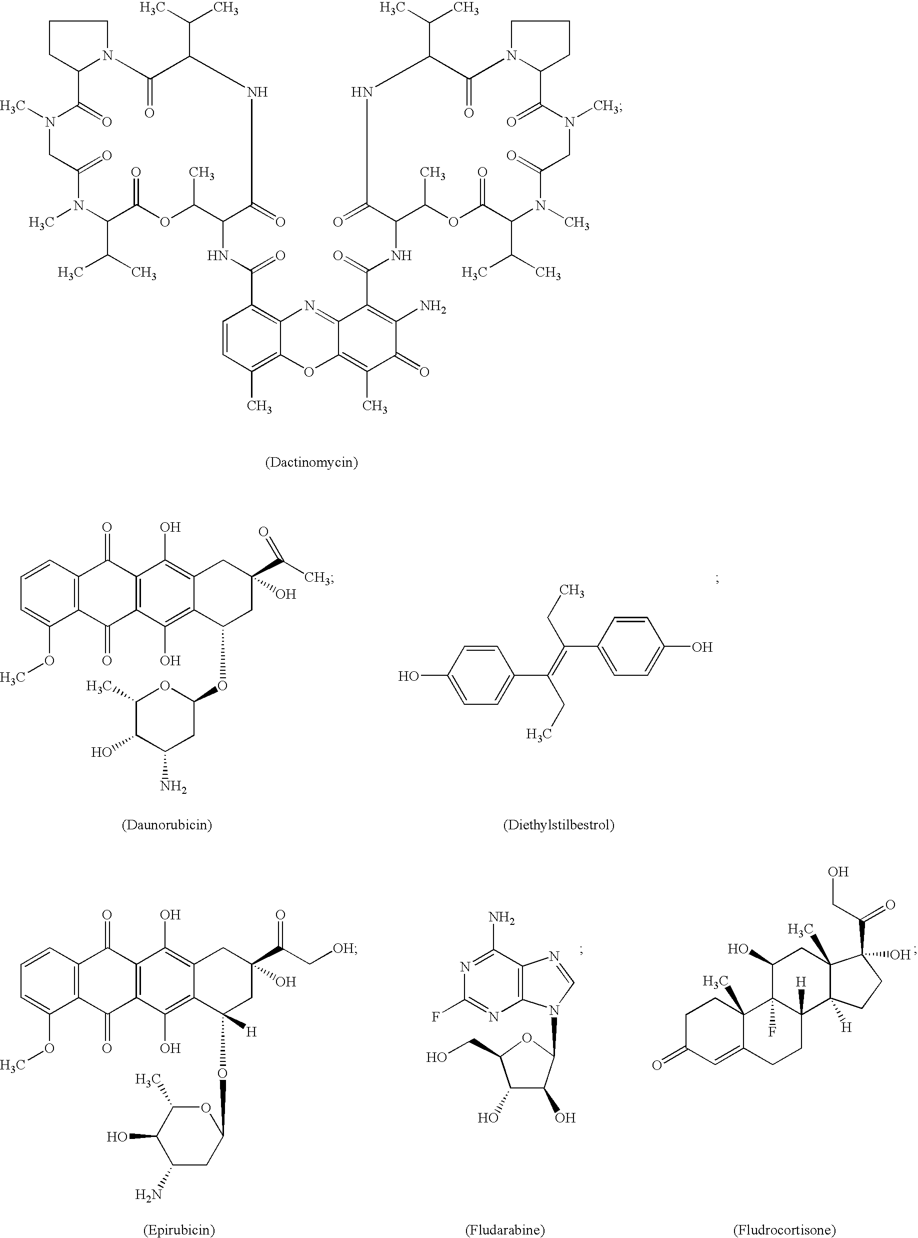 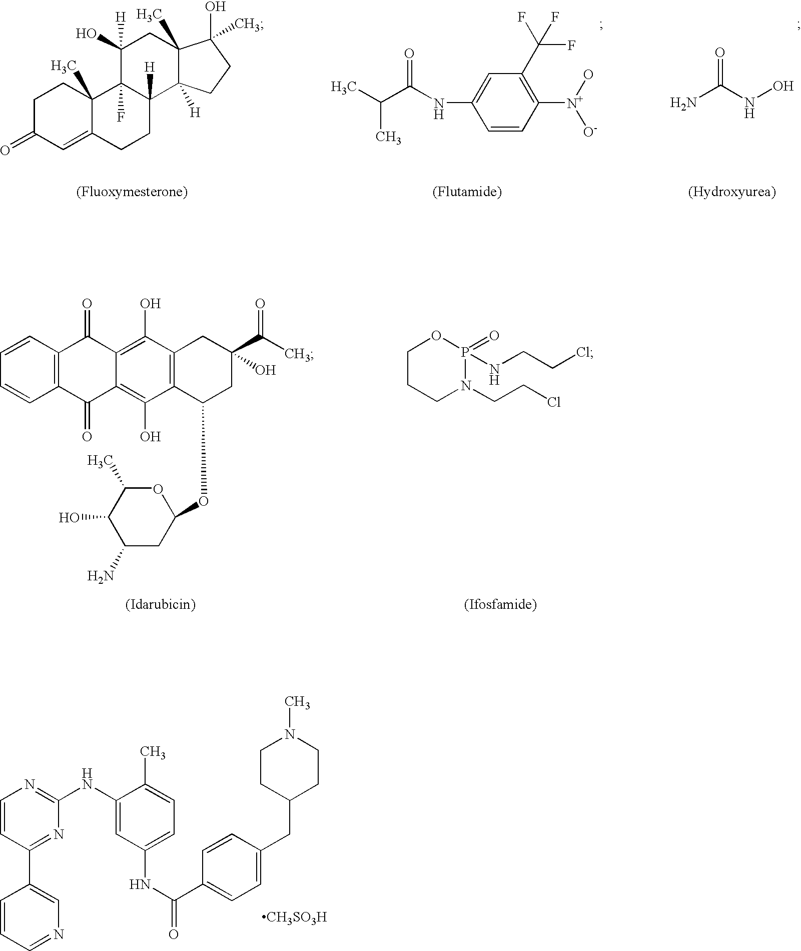
(Imatinib; sold as Gleevec® by Novartis Pharmaceuticals Corporation; East Hanover, N.J.); 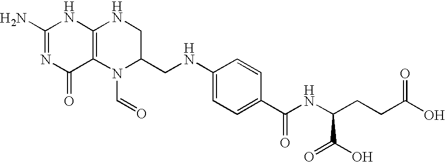
(Leucovorin); 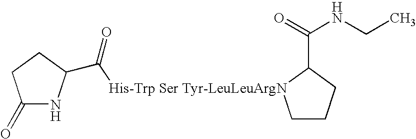
(Leuprolide; 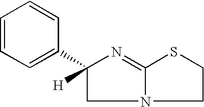
(Levamisole); 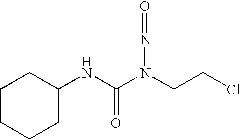
(Lomustine); 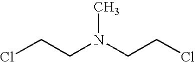
(Mechlorethamine); 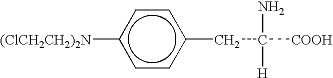
(Melphalan; sold as Alkeran® by Celgene Corporation; Warren, N.J.); 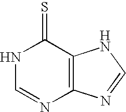
(Mercaptopurine); 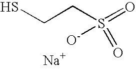
(Mesna); 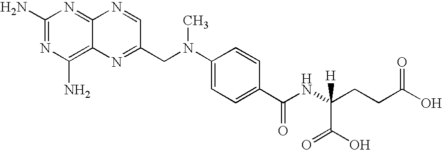
(Methotrexate); 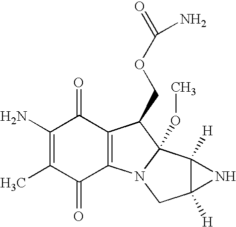
(Mitomycin); 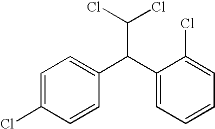
(Mitotane); 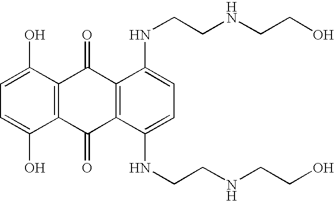
(Mitoxantrone); 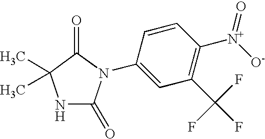
(Nilutamide); octreotide (L-Cysteinamide, D-phenylalanyl-L-cysteinyl-L-phenylalanyl-D-tryptophyl-L-lysyl-L-threonyl-N-[2-hydroxy-1-(hydroxymethyl)propyl]-, cyclic(2₁₃ 7)-disulfide; [R R*,R*)];

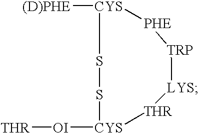

Katz et al., Clin Pharm. 8(4):255-73 (1989); sold as Sandostatin LAR® Depot; Novartis Pharm. Corp; E. Hanover, N.J.); oxaliplatin ( 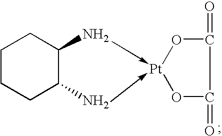
sold as Eloxatin™ by Sanofi-Synthelabo Inc.; New York, N.Y.); 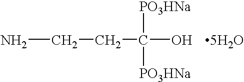
(Pamidronate; sold as Aredia® by Novartis Pharmaceuticals Corporation; East Hanover, N.J.); 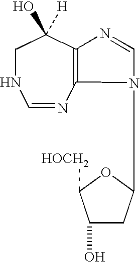
(Pentostatin; sold as Nipent® by Supergen; Dublin, Calif.); 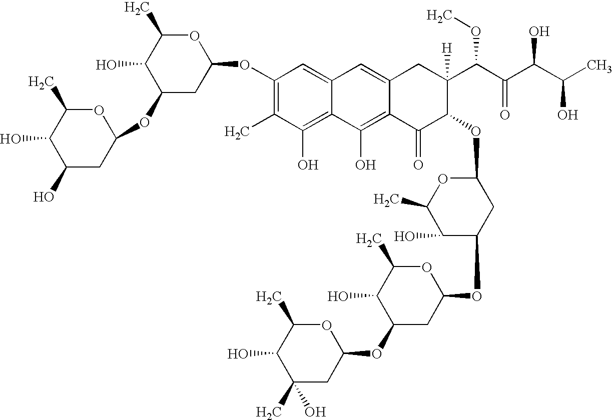
(Plicamycin); 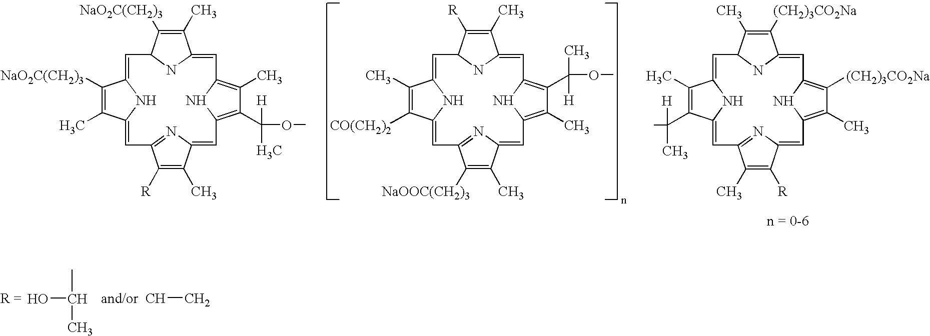
(Porfimer; sold as Photofrin® by Axcan Scandipharm Inc.; Birmingham, Ala.); 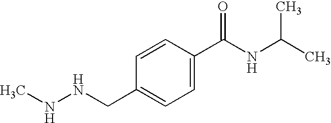
(Procarbazine); 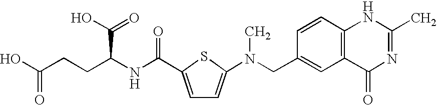
(Raltitrexed); Rituximab (sold as Rituxan® by Genentech, Inc.; South San Francisco, Calif.); 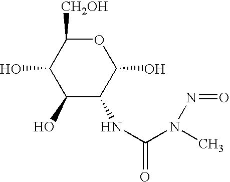
(Streptozocin); 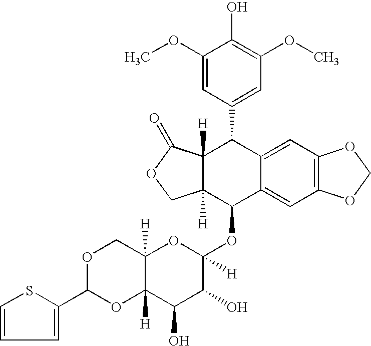
(Teniposide); 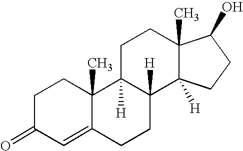
(Testosterone); 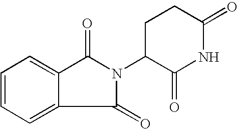
(Thalidomide); 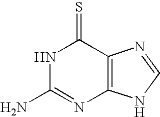
(Thioguanine); 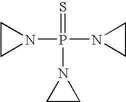
(Thiotepa); 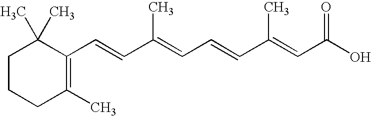
(Tretinoin); 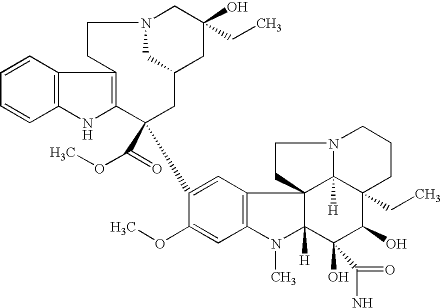
(Vindesine) or 13-cis-retinoic acid

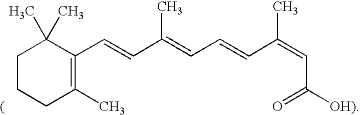

In an embodiment of the invention, the further chemotherapeutic agent is one or more of any of phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin, diftitox, gefitinib, bortezimib, paclitaxel, docetaxel, epithilone B, BMS-247550 (see e.g., Lee et al., Clin. Cancer Res. 7:1429-1437 (2001)), BMS-310705, droloxifene (3-hydroxytamoxifen), 4-hydroxytamoxifen, pipendoxifene, ERA-923, arzoxifene, fulvestrant, acolbifene, lasofoxifene (CP-336156), idoxifene, TSE-424, HMR-3339, ZK186619, topotecan, PTK787/ZK 222584 (Thomas et al., Semin Oncol. 30(3 Suppl 6):32-8 (2003)), the humanized anti-VEGF antibody Bevacizumab, VX-745 (Haddad, Curr Opin. Investig. Drugs 2(8):1070-6 (2001)), PD 184352 (Sebolt-Leopold, et al., Nature Med. 5: 810-816 (1999)), rapamycin, CCI-779 (Sehgal et al., Med. Res. Rev., 14:1-22 (1994); Elit, Curr. Opin. Investig. Drugs 3(8):1249-53 (2002)), LY294002, LY292223, LY292696, LY293684, LY293646 (Vlahos et al., J. Biol. Chem. 269(7): 5241-5248 (1994)), wortmannin, BAY-43-9006, (Wilhelm et al., Curr, Pharm. Des. 8:2255-2257 (2002)), ZM336372, L-779,450, any Raf inhibitor disclosed in Lowinger et al, Curr. Pharm Des. 8:2269-2278 (2002); flavopiridol (L86-8275/HMR 1275; Senderowicz, Oncogene 19(56): 6600-6606 (2000)) or UCN-01 (7-hydroxy staurosporine; Senderowicz, Oncogene 19(56): 6600-6606 (2000)).

In an embodiment of the invention, the further chemotherapeutic agent is one or more of any of the compounds set forth in U.S. Pat. No. 5,656,655, which discloses styryl substituted heteroaryl EGFR inhibitors; in U.S. Pat. No. 5,646,153 which discloses bis mono and/or bicyclic aryl heteroaryl carbocyclic and heterocarbocyclic EGFR and PDGFR inhibitors; in U.S. Pat. No. 5,679,683 which discloses tricyclic pyrimidine compounds that inhibit the EGFR; in U.S. Pat. No. 5,616,582 which discloses quinazoline derivatives that have receptor tyrosine kinase inhibitory activity; in Fry et al., Science 265 1093-1095 (1994) which discloses a compound having a structure that inhibits EGFR (see FIG. 1 of Fry et al.); in U.S. Pat. No. 5,196,446 which discloses heteroarylethenediyl or heteroarylethenediylaryl compounds that inhibit EGFR; in Panek, et al., Journal of Pharmacology and Experimental Therapeutics 283: 1433-1444 (1997) which disclose a compound identified as PD166285 that inhibits the EGFR, PDGFR, and FGFR families of receptors-PD166285 is identified as 6-(2,6-dichlorophenyl)-2-(4-(2-diethylaminoethoxy)phenylamino)-8-methyl-8H-pyrido(2,3-d)pyrimidin-7-one.

In an embodiment of the invention, the further chemotherapeutic agent is one or more of any of pegylated or unpegylated interferon alfa-2a, pegylated or unpegylated interferon alfa-2b, pegylated or unpegylated interferon alfa-2c, pegylated or unpegylated interferon alfa n-1, pegylated or unpegylated interferon alfa n-3 and pegylated, unpegylated consensus interferon or albumin-interferon-alpha.

The scope of the present invention also includes methods of treatment comprising administering an IGF1R inhibitor in association with one or more antiemetics including, but not limited to, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co,; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by GlaxoSmithKline; Research Triangle Park, N.C.), granisetron (sold as Kytril® by Hoffmann-La Roche Inc.; Nutley, N.J.), ondansetron (sold as Zofran® by by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Compositions comprising an antiemetic are useful for preventing or treating nausea; a common side effect of anti-cancer chemotherapy. Accordingly, the present invention also includes methods for treating or preventing cancer in a subject by administering an IGF1R inhibitor optionally in association with one or more other chemotherapeutic agents (e.g., as described herein) and/or optionally in association with one or more antiemetics.

The present invention further comprises methods of treatment comprising administering an IGF1R inhibitor in association with a therapeutic procedure such as surgical tumorectomy or anti-cancer radiation treatment; optionally in association with a further chemotherapeutic agent and/or antiemetic, for example, as set forth above.

As discussed above, the present invention comprises methods wherein an IGF1R inhibitor is administered in association with a further anti-cancer chemotherapeutic agent or procedure. In an embodiment of the invention, the term “in association with” indicates that the components of the combinations of the invention are be formulated into a single composition for simultaneous delivery or formulated separately into two or more compositions (e.g., a kit). Furthermore, each component of a combination of the invention can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., orally, intravenously, subcutaneously).

### Determination of IGFBP2 Levels

IGFBP2 levels may be measured by any of several methods which are very well known in the art; some of which are discussed infra.

IGFBP2 can be quantitated, for example, by simply hiring or contracting with a commercial laboratory to perform the assay. Alternatively, the practitioner can perform the assay himself. In an embodiment of the invention, IGFBP2 is quantitated by a radioimmunoassay (RIA) (see e.g., Smith et al., J. Clin. Endocrin. Metab. 77(5): 1294-1299 (1993); Cohen et al., J. Clin. Endocrin. Metab. 76(4). 1031-1035 (1993); Dawczynski et al., Bone Marrow Transplant, 37:589-594 (2006); and Clemmons et al., J. Clin. Endocrin. Metab. 73:727-733 (1991)), western blot, western ligand blot (WLB) or by ELISA (enzyme linked immunosorbent assay). For example, in an embodiment of the invention, IGFBP2 in a sample of a patient's tumor tissue, plasma, blood or serum is quantitated.

In an embodiment of the invention, western ligand blots are performed as follows: Samples (2.5 μL) are electrophoresed on 10% polyacryl-amide-sodium dodecyl sulfate (SDS-PAGE) gels (e.g., 10, 12 or 14%), electroblotted onto nitrocellulose, incubated with [¹²⁵I]-IGF-I, and exposed to film, e.g., for about 5-10 days. Each lane of the autoradiograph is developed, scanned and analyzed by densitometer.

In an embodiment of the invention, western blots are performed as follows; A sample is electrophoresed on a polyacrylamide-sodium dodecyl sulfate (SDS-PAGE) gel (e.g., 10, 12 or 14%) and transferred onto nitrocellulose or some other suitable membrane. The membrane is then incubated with a primary antibody which binds to the protein being evaluated, optionally washed and then incubated with a detectably labeled secondary antibody that binds to the primary antibody and optionally washed again. The presence of the secondary antibody is then detected. For example, if the secondary antibody is labeled with a chemilluminescence label, the membrane is exposed to film and then the film is developed. In an embodiment of the invention, each lane of the autoradiograph is scanned and analyzed by densitometer.

In an embodiment of the invention, a RIA is performed as follows: IGFBP2 is iodinated by adding 0.5 mCi [¹²⁵I]-sodium iodide to 0.1 ml, 0.5M phosphate buffer, pH7.5. Chloramines T (60 μM) is added and the mixture is incubated for 3 minutes. The percentage iodination is determined by adding an aliquot of the mixture to 1 ml 10% bovine serum albumin followed by precipitation with an equal volume of ice cold 20% trichloroacetic acid. Additional chloramines-T is added when necessary to achieve 65% trichloroacetic acid precipitability and the reaction is terminated by the addition of sodium metabisulfite (final concentration 120 μM). The mixture is purified by Sepadex G-75 chromotagraphy in 0.01M phosphate buffer pH7.5. Non-specific binding can be determined in the presence of 100 ng/ml pure human IGFBP2. [¹²⁵I]-IGFBP2 can be stored in siliconized tubes in 0.2% BSA at −70° C. RIA can be performed by using 0.5 ml 0.03 M phosphate buffer, pH 7.4, containing 0.01M EDTA, 0.01 Tween-20 and 0.1% bovine serum albumin. Test samples can be added by diluting the serum or plasma 1:10 then adding volumes of 10-40 μl and assays can, in an embodiment of the invention, be performed in duplicate. After incubation for 24 hours at 4° C., [¹²⁵I]-IGFBP2 (e.g., about 16000 cpm/tube) can be added and the incubation continued for another 16 hours. Four microliters of anti-IGFBP2 antiserum (e.g., rabbit antiserum) and a secondary antibody can be added and incubation can be continued for 1 hour at 4° C. followed by 2 ml normal rabbit serum followed by a final 1 hour at 4° C. Bound and free [¹²⁵I]-IGFBP2 can be separated by centrifugation at 9000×g for 30 minutes and bound [¹²⁵I]-IGFBP2 can be determined by γ-spectrometer. All unknown results can be read against a standard curve that contains e.g., 50 pg and 1 ng/tube of pure IGFBP2 (see Clemmons et al., J. Clin. Endocrin. Metab 73(4):727-733 (1991)).

Radioimmunoassays are based on the reaction between an antibody and an antigen whose concentration has to be quantified. A known quantity of radioactively labeled IGFBP2 is mixed with a dilution series of “cold” IGFBP2. The dilution series is brought to reaction with a fixed amount of anti-IGFBP2 antibody. Since cold and radioactively labeled IGFBP2 antigens compete with each other for the antibody binding sites, a high concentration of cold IGFBP2 will result in little radioactive IGFBP2 antigen bound to the antibody and vice versa. After a fixed time, a secondary antibody directed against the first anti-IGFBP2 antibody is used which leads to the formation of large complexes which upon centrifugation are counted with a radioactive counter. This fraction contains the “cold” and the radioactive antigen which has bound to the specific antibody, while the supernatant in the centrifugate contains the unbound antigen. The serially diluted probes yield points on a curve relating radioactive counts to the concentration of cold IGFBP2 antigen: a so-called (cold) reference curve. Using this reference curve, an unknown quantity of IGFBP2 antigen can be quantified by identification of the radioactive counts in the centrifugate and use of the reference curve which yields the unknown antigen concentration.

In an embodiment of the invention, an ELISA assay employs an antibody specific for human IGFBP2 coated on a 96-well plate. Standards and samples are pipetted into the wells and IGFBP2 present in a sample is bound to the wells by the immobilized antibody. The wells are washed and biotinylated anti-IGFBP2 antibody is added. After washing away unbound biotinylated antibody, HRP-conjugated streptavidin is pipetted to the wells. The wells are again washed, a TMB substrate solution is added to the wells and color develops in proportion to the amount of IGFBP2 bound. The Stop Solution changes the color from blue to yellow, and the intensity of the color is measured at 450 nm (see e.g., Human IGFBP-2 ELISA Kit from RayBiotech, Inc.; Norcross, Ga.; and Angervo M et al., Biochemical and Biophysical Research Communications 189: 1177-83 (1992); Kratz et al., Experimental Cell Research 202: 381-5 (1992); and Frost et al., Journal of Biological Chemistry 266: 18082-8 (1991)). A standard ELISA curve using known concentrations of IGFBP2 can be plotted and the concentration of IGFBP2 in the unknown sample (e.g., the serum of a patient) can be determined by comparing the signal observed therein with the signal observed in the standard.

Anti-IGFBP2 antibodies that can be used in an assay of the invention can be purchased commercially or easily generated by a practitioner using conventional methods known in the art. See e.g. Bourner et al., J. Cell. Biochem. 48:215-226 (1992) and Camacho-Hobner, C., et al., J. Biol. Chem 267:11949-11956 (1992) which describe the rabbit polyclonal anti-IGFBP2 antibody, ab4244 (Abcam, Inc.; Cambridge, Mass.). See e.g., Allander et al., Am. J. Pathology 161: 1587-1595 (2002) describing the goat anti-IGFBP2 Cruz Biotechnology, Inc.; Santa Cruz, Calif.). See e.g., Suzuki, et al., J. Comp. Neurol. 482: 74-84 (2005); La et al. Endocrinology 145: 3443-3450 (2004); and Hoeflich et al., Biochem Biophys Res Commun. 324: 705-710 (2004) describing the anti-IGFBP2 goat polyclonal IgG, M-18 (Santa Cruz Biotechnology, Inc.; Santa Cruz, Calif.). See also, the anti-IGFBP2 rabbit polyclonal IgG, H-75 and the anti-IGFBP2 mouse monoclonal IgG1, C-10 (Santa Cruz Biotechnology, Inc.; Santa Cruz, Calif.).

### Diagnostics and Patient Selection

The present invention provides a method for diagnosing the presence of cancer or any other medical condition mediated by IGF1R expression or activity in a patient, for example, wherein the condition is cancer and the cancerous or tumor cells express IGF1R. The diagnostic method comprises determining if the patient exhibits elevated levels of IGFBP2. If the patient is determined to exhibit elevated IGFBP2, then the patient is determined to suffer from cancer or some other medical disorder mediated by IGF1R expression and/or activity. In an embodiment of the invention, the medical condition is osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotologicai malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis or Bechet's disease. In an embodiment of the invention, the diagnosis of cancer in the patient as set forth above is confirmed, e.g., using conventional techniques. For example, the presence of a tumor can be confirmed by X-ray, MRI, CT scan, PET scan, palpation, ultrasonography or surgery.

In an embodiment of the invention, diagnosis of the presence of cancer in a patient is followed by treatment with a therapeutically effective amount of an IGF1R inhibitor or combination thereof with an anti-cancer therapeutic agent or anti-cancer procedure as set forth herein.

In an embodiment of the invention, normal or non-elevated levels of human IGFBP2 range from about 48-340 ng/ml (e.g., about 241 ng/ml±about 28 ng/ml or±about 10%; or about 150 ng/ml±61 ng/ml). In an embodiment of the invention, the human IGFBP2 level of a pediatric patient (e.g., about 2 months to about 1 year old) is about 263 ng/ml (in an embodiment ±81 ng/ml). In an embodiment of the invention, the human IGFBP2 level of a pediatric patient (e.g., 15-18 years old) is about 136 ng/ml (in an embodiment ±38 ng/ml).

In an embodiment of the invention, the normal IGFBP2 level is as determined by western ligand blots (WLB) or by radioimmunoassay (RIA). In an embodiment of the invention, IGFBP2 is measured in any bodily fluid of the patient, for example, blood, plasma, serum or tumor tissue.

In an embodiment of the invention, elevated or supranormal levels of IGFBP2 in a patient are any level that a practitioner of ordinary skill in the art would recognize as such. In an embodiment of the invention, an elevated or supranormal level of IGFBP2 which is over the range of 48-340 ng/ml or over about 241 ng/ml (e.g., as determined by WLB or RIA). In an embodiment of the invention, an elevated or supranormal level of IGFBP2 is at least about 50% to about 100% (e.g., 50%, 55%, 60%, 65%, 70%, 75%, 80%, 85%, 90%, 95%, 100%, 200%, 300%, 400% or 500%) higher than a normal level. In an embodiment of the invention, an elevated or supranormal level of IGFBP2 level is determined with respect to a particular patient. In such an embodiment a patient's IGFBP2 level is measured at an initial time point and measured at one or more points in the future. If one or more of the future measurements is higher than the initial measurement, the patient is determined to exhibit an elevated or supranormal IGFBP2 level.

The present invention further provides a method for selecting a patient suffering from a cancer likely to be responsive to an IGF1R inhibitor. In an embodiment of the invention, the cells of the cancerous tumor express IGF1R and the patient exhibits elevated IGFBP2 levels. If the patient is identified to possess a tumor that expresses IGF1R or if the tumor is known to express IGF1R and if the patient exhibits elevated or supranormal IGFBP2 levels or possesses a tumor known to be associated with elevated IGFBP2, then the patient is selected for treatment with an IGF1R inhibitor, e.g., as set forth herein.

### Dosage and Method for Monitoring and Evaluating IGF1R Inhibitor Therapy

The present invention provides methods for quickly and conveniently evaluating various aspects of a given IGF1R inhibitor therapeutic regimen. For example, the present invention provides a method for monitoring the effect of an IGF1R inhibitor on IGFBP2 concentration in the body of a subject administered said inhibitor comprising measuring IGFBP2 levels in the body of the subject over time. For example, in a more specific embodiment of the invention, an initial, baseline IGFBP2 level is measured before any dosage of IGF1R inhibitor is given. Following the commencement of an IGF1R treatment regimen, one or more measurements of IGFBP2 levels in the body of the subject (e.g., in the blood or plasma of the subject) are measured and compared.

For example, the present invention comprises a method for monitoring the effect of an IGF1R inhibitor (e.g., anti-IGF1R antibody) on the IGF1 receptor or any component of the IGF1R pathway in the body of a subject administered said inhibitor comprising evaluating IGFBP2 levels in the body of the subject over time; wherein the inhibitor is determined to inhibit the receptor or pathway if IGFBP2 levels are observed to decrease over time (e.g., by at least 51%) following said administration; or wherein the inhibitor is determined not to inhibit the receptor or pathway if IGFBP2 levels are not observed to decrease over time (e.g., by at least 51%) following said administration. In an embodiment of the invention, an initial, baseline IGFBP2 level is measured before any dosage of IGF1R inhibitor is given. Following the commencement of an IGF1R inhibitor treatment regimen, one or more measurements of IGFBP2 levels in the body of the subject (e.g., in the blood, serum or plasma of the subject) are measured and compared and the effect of the inhibitor on the receptor or pathway is then determined. In an embodiment of the invention, the level of IGFBP2 decrease or increase during the course of an IGF1R inhibitor regimen, is evaluated by a clinician in view oft e.g., the particularities of the subject's medical condition, status, sensitivities and history and weighed as one factor (e.g., among many) when deciding if the regimen is yielding an acceptable therapeutic effect. For example, in an embodiment of the invention, the IGFBP2 level is evaluated qualitatively for the purpose of gauging the sufficiency of the regimen. When monitoring the effect of an IGF1R inhibitor on the IGF1 receptor is mentioned, this includes monitoring the effect of the inhibitor on the receptor itself as well as on any member of the IGF1R signaling pathway.

The present invention further includes a method for evaluating dosage of an IGF1R inhibitor (e.g., the amount of the dosage and/or the frequency of the dosage and/or the mode of administration of the dosage) administered to a subject comprising administering a dose of said inhibitor to said subject and evaluating IGFBP2 levels in the body of the subject over time; wherein said dosage is determined to be insufficient if IGFBP2 levels are not observed to decrease (e.g., by at least 51%) over time following said administration; or wherein said dosage is determined to be sufficient if IGFBP2 levels are observed to decrease over time (e.g., by at least 51%) following said administration. In a more specific embodiment of the invention, an initial, baseline IGFBP2 level is measured before any dosage of IGF1R inhibitor is given. Following the commencement of an IGF1R treatment regimen, one or more measurements of IGFBP2 levels in the body of the subject (e.g., in the blood or plasma of the subject) are measured and compared and the sufficiency of the dosage is then determined. In an embodiment of the invention, an IGF1R inhibitor dosage is adjusted up or down so that IGFBP2 levels, when elevated in a subject receiving the inhibitor, return to normal levels. Normal, low and elevated levels of IGFBP2 are known to any practitioner of ordinary skill in the art and are also discussed herein.

The scope of the present invention also includes a method for determining if a subject has a medical condition that is responsive to an IGF1R inhibitor comprising administering said inhibitor to said subject and evaluating IGFBP2 levels in the body of the subject over time; wherein said condition is determined to be unresponsive to said inhibitor if the IGFBP2 levels are not observed to decrease over time following said administration. If the subject proves to be essentially unresponsive to an IGF1R inhibitor, for example wherein IGFBP2 levels, and, thus, the IGF1R pathway itself does not decrease in response to the inhibitor, then the inhibitor therapy may be discontinued. Alternatively, the dosage can be increased so as to determine if the IGF1R pathway becomes responsive upon exposure to greater dosages. In a more specific embodiment of the invention, an initial, baseline IGFBP2 level is measured before any dosage of IGF1R inhibitor is given. Following the commencement of an IGF1R treatment regimen, one or more measurements of IGFBP2 levels in the body of the subject (e.g., in the blood or plasma of the subject) are measured and compared and whether the medical condition in the subject is responsive or unresponsive is then determined.

The present invention also provides a method for selecting a dose of an IGF1R inhibitor comprising administering a dose of said inhibitor to a subject with a medical condition mediated by IGF1R expression or activity and evaluating IGFBP2 levels in the body of the subject; wherein said dosage is selected if IGFBP2 levels are observed to decrease by at least 51% of an IGFBP2 level measured prior to first administration of said inhibitor following said administration. In an embodiment of the invention, the method comprises (i) measuring an IGFBP2 level in the body of said subject before treatment with said inhibitor; (ii) administering one or more doses (i.e., doses of a single given amount such as 10 mg/kg given one or more times) of said inhibitor to said subject; (iii) measuring an IGFBP2 level in the body of said subject following said administration; (iv) comparing the level of IGFBP2 measured in step (i) with the level of IGFBP2 measured in step (iii); wherein said dose is selected if IGFBP2 levels are observed to decrease by at least 51% of an IGFBP2 level measured prior to first administration of said inhibitor following said administration. For example, if the dosage is selected, treatment of the subject at the selected dosage is continued.

The present invention also provides a method for treating a medical condition, in a subject, mediated by IGF1R expression or activity comprising (i) measuring an IGFBP2 level in the body of said subject prior to any administration of an IGF1R inhibitor; (ii) administering one or more doses of an IGF1R inhibitor to said subject; (iii) measuring an IGFBP2 level in the body of said subject following said administration; (iv) comparing the level of IGFBP2 measured in step (i) with the level of IGFBP2 measured in step (iii), and (v) increasing dosage of said inhibitor if the IGFBP2 level does not decrease by at least 51% following said administration; or maintaining dosage if the IGFBP2 level does decrease by at least 51% following said administration.

In an embodiment of the invention, the dosage of said inhibitor is determined to be insufficient or is not selected; or the inhibitor is determined to not inhibit IGF1R or its pathway; or the subject is not determined to be responsive to the IGF1R inhibitor if IGFBP2 levels are determined not to decrease by at least about 51% of the initial, pre-treatment IGFBP2 level. Optionally, the dosage is increased if the IGFBP2 levels do not drop sufficiently. For example, the amount of dosage or the frequency of dosage may be increased if said dosage is determined to be insufficient. In an embodiment of the invention, the initial dosage that is evaluated is between about 0.3 mg/kg and 20 mg/kg (e.g., 1 mg/kg, 3 mg/kg, 10 mg/kg), once a week.

If, for example, IGFBP2 levels do decrease by at least about 51%, and subsequently increase above about 51%, then dosage of the IGF1R inhibitor may be increased. If the increased dosage leads to a decrease in IGFBP2 levels to the originally set 51% target, that increase dosage may then be selected or determined to be sufficient and maintained.

Dosage of an IGF1R inhibitor may, in an embodiment of any of the inventions set forth herein, be decreased if IGFBP2 levels decrease significantly more than 51%; for example, if a physician determines that the IGFBP2 levels have decreased to a dangerously low level.

In connection with any of the methods discussed herein, effects of IGF1 receptor inhibitors on the IGF1 receptor pathway are evaluated. The effects on the pathway include, but are not limited to, modulation of IGF1R kinase activity, Sos-1, Ras, Raf, Mek, Erk, PKA, PI3 kinase activity, Grb2 activity, AKT kinase activity or MAP kinase activity such that a reduction of cell (e.g., malignant cell) growth or survival or an increase in cellular apoptosis (e.g., of malignant cells) results wherein IGFBP2 levels are the marker for modulation of the pathway.

In an embodiment of the invention, an IGF1R inhibitor is administered to a patient at a “therapeutically effective dosage” or “therapeutically effective amount” which preferably inhibits a disease or condition (e.g., tumor growth) to any extent As discussed herein, the proper dosage can be adjusted according to observations made by the clinician, physician or veterinarian. In an embodiment of the invention, the term “therapeutically effective amount” or “therapeutically effective dosage” means that amount or dosage of an IGF1R inhibitor (e.g., an anti-IGF1R antibody or antigen-binding fragment thereof) that will elicit a biological or medical response of a tissue, system, subject or host that is being sought by the administrator (such as a researcher, doctor or veterinarian) which includes survival of the subject (e.g., for 3 months, 6 months, 1 year, 2 years, 3 years, 4 years or 5 years after completing an IGF1R inhibitor regimen) and/or any measurable alleviation of the signs, symptoms and/or clinical indicia of cancer (e.g., tumor growth or survival) and/or the prevention, slowing or halting of progression or metastasis of cancer to any degree. Furthermore, in embodiment of the invention, an inhibitor or its dose is evaluated to determine if IGF1R is “sufficiently” inhibited; the effect sought through evaluation of IGFBP2 includes any of the biological or medical responses discussed above. One of ordinary skill in the art would be able to determine such amounts based on such factors as the subject's size, the severity of the subjects symptoms, and the particular composition or route of administration selected.

In an embodiment of the invention, administration of IGF1R inhibitor is by injection proximal to the site of the target (e.g., tumor). In an embodiment, a therapeutically effective daily dose of IGF1R inhibitor or pharmaceutical composition thereof is administered as two, three, four, five, six or more sub-doses administered separately at appropriate intervals throughout the day. In an embodiment, a “therapeutically effective” dosage of any anti-IGF1R antibody (e.g., mature 19D12/15H12 LCF/HCA, e.g., see CAS registry no. 93423544-6) is in the range of about 0.3 mg/kg (body weight) to about 20 mg/kg (e.g., 0.3 mg/kg, 1 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg or 20 mg/kg) about once per week to about once every 3 weeks (e.g., about once every 1 week or once every 2 weeks or once every 3 weeks). In an embodiment, a “therapeutically effective dosage” of a chemotherapeutic agent (e.g., an IGF1R inhibitor) is, whenever possible, as set forth in the Physicians' Desk Reference 2003 (Thomson Healthcare; 57^{th} edition (Nov. 1, 2002)) which is herein incorporated by reference. For example, in an embodiment of the invention, a therapeutically effective dosage of NVP-ADW-742 is about 1 mg/kg/day to about 50 mg/kg/day (e.g., 5 mg/kg/day, 10 mg/kg/day, 15 mg/kg/day, 20 mg/kg/day, 25 mg/kg/day, 30 mg/kg/day, 35 mg/kg/day, 40 mg/kg/day, 45 mg/kg/day).

A physician or clinician can, optionally, also adjust the dosage of an IGF1R inhibitor using conventional techniques and clinical indicia in addition to IGFBP2 levels as discussed herein; such additional techniques and indicia are discussed below. For example, a clinician can evaluate the actual size and progress of the tumor being treated. The size and progress of a tumor can also be easily determined, for example, by X-ray, magnetic resonance imaging (MRI) or visually in a surgical procedure. In general, tumor size and proliferation can be measured by use of a thymidine PET scan (see e.g., Wells et al., Clin. Oncol. 8: 7-14 (1996)). Generally, the thymidine PET scan includes the injection of a radioactive tracer, such as [2-¹¹C]-thymidine, followed by a PET scan of the patient's body (Vander Borght et al., Gastroenterology 101: 794-799, 1991; Vander Borght et al., J. Radiat. Appl. Instrum. Part A, 42: 103-104 (1991)). Other tracers that can be used include [¹⁸F]-FDG (18-fluorodeoxyglucose), [¹²⁴I]IUdR (5-[124]iodo-2′-deoxyuridne), [⁷⁶Br]BrdUrd (Bromodeoxyuridine), [¹⁸F]FLT (3′-deoxy-3′fluorothymidine) or [¹¹C]FMAU (2′-fluoro-5-methyl-1-β-D-arabinofuranosyluracil).

For example, neuroblastoma progress can also be monitored, by a physician or veterinarian, by a variety of methods, and the dosing regimen can be altered accordingly. Methods by which to monitor neuroblastoma include, for example, CT scan (e.g., to monitor tumor size), MRI scan (e.g., to monitor tumor size), chest X-ray (e.g., to monitor tumor size), bone scan, bone marrow biopsy (e.g., to check for metastasis to the bone marrow), hormone tests (levels of hormones like epinephrine), complete blood test (CBC) (e.g., to test for anemia or other abnormality), testing for catecholamines (a neuroblastoma tumor marker) in the urine or blood, a 24 hour urine test for check for homovanillic acid (HMA) or vanillyl mandelic acid (VMA) levels (neuroblastoma markers) and an MIBG scan (scan for injected I¹²³-labeled metaiodobetaguanidine; e.g., to monitor adrenal tumors).

For example, rhabdomyosarcoma progress can also be monitored, by the physician or veterinarian, by a variety of methods, and the dosing regimen can be altered accordingly. Methods by which to monitor rhabdomyosarcoma include, for example tumor biopsy, CT scan (e.g., to monitor tumor size), MRI scan (e.g., to monitor tumor size), CT scan of the chest (e.g., to monitor metastases), bone scan (e.g., to monitor metastases), bone marrow biopsy (e.g., to monitor metastases), spinal tap (e.g., to check for metastasis into the brain) and a thorough physical exam.

For example, osteosarcoma progress can also be monitored, by the physician or veterinarian, by a variety of methods, and the dosing regimen can be altered accordingly. Methods by which to monitor osteosarcoma include, for example, X-ray of the affected area or of the chest (e.g., to check for spread to the lungs), CT scan of the affected area, blood tests (e.g., to measure alkaline phosphatase levels), computer tomography scan (CT) of the chest to see if the cancer has spread to the lungs, open biopsy, or a bone scan to see if the cancer has spread to other bones.

For example, Wilm's cancer progress can also be monitored, by the physician or veterinarian, by a variety of methods, and the dosing regimen can be altered accordingly. Methods by which to monitor Wilm's cancer include abdominal computer tomography scan (CT), abdominal ultrasound, blood and urine tests to evaluate kidney and liver function, chest X-ray to check for metastasis, magnetic resonance imaging (MRI), blood tests and urinalysis to assay kidney function and biopsy.

For example, pancreatic cancer progress can also be monitored, by the physician or veterinarian, by a variety of methods, and the dosing regimen can be altered accordingly. Methods by which to monitor pancreatic cancer include blood tests to check for tumor markers CA 19-9 and/or carcinoembryonic antigen (CEA), an upper GI series (e.g., a barium swallow), endoscopic ultrasonography; endoscopic retrograde cholangiopancreatography (an X-ray of the pancreatic duct and bile ducts); percutaneous transhepatic cholangiography (an X-ray of the bile duct), abdominal ultrasound imaging or abdominal computer tomography scan (CT).

For example, breast cancer progress can also be monitored, by the physician or veterinarian, by a variety of methods, and the dosing regimen can be altered accordingly. Methods by which to monitor breast cancer include mammography, aspiration or needle biopsy or palpation.

For example, colorectal cancer progress can also be monitored, by the physician or veterinarian, by a variety of methods, and the dosing regimen can be altered accordingly. Methods by which to monitor colorectal cancer include computer tomography scan (CT), MRI scan, chest X-ray, PET scan, fecal occult blood tests (FOBTs), flexible proctosigmoidoscopy, total colonoscopy, and barium enema.

For example, gastric cancer progress can also be monitored, by the physician or veterinarian, by a variety of methods, and the dosing regimen can be altered accordingly. Methods by which to monitor gastric cancer include esophagogastroduodenoscopy (EGD), double-contrast barium swallow, endoscopic biopsy, computed tomographic (CT) scanning, magnetic resonance imagine (MRI) or endoscopic ultrasonography (EUS).

For example, bladder cancer progress can also be monitored, by the physician or veterinarian, by a variety of methods, and the dosing regimen can be altered accordingly. Methods by which to monitor bladder cancer include urinalysis to detect elevated levels of tumor markers (e.g., nuclear matrix protein (NMP22)) in the urine, urinalysis to detect microscopic hematuria, urine cytology to detect cancer cells by examining cells flushed from the bladder during urination, bladder cystoscopy, intravenous pyelogram (IVP), retrograde pyelography, chest X ray to detect metastasis, computed tomography (CT), bone scan, MRI scan, PET scan or biopsy.

For example, lung cancer progress can also be monitored, by the physician or veterinarian, by a variety of methods, and the dosing regimen can be altered accordingly. Methods by which to monitor lung cancer include chest X-ray, CT scan, low-dose helical CT scan (or spiral CT scan), MRI scan, PET scan, bone scan, sputum cytology, bronchoscopy, mediastinoscopy, biopsy (e.g., needle or surgical), thoracentesis or blood tests to detect PTH (parathyroid hormone), CEA (carcinogenic antigen) or CYFRA21-1 (cytokeratin fragment 19).

For example, prostate cancer progress can also be monitored, by the physician or veterinarian, by a variety of methods, and the dosing regimen can be altered accordingly. Methods by which to monitor prostate cancer include digital rectal examination, transrectal ultrasound, blood tests taken to check the levels of prostate specific antigen (PSA) and prostatic acid phosphatase (PAP), biopsy, bone scan and CT scan.

For example, cervical cancer progress can also be monitored, by the physician or veterinarian, by a variety of methods, and the dosing regimen can be altered accordingly. Methods by which to monitor cervical cancer include PAP smear, pelvic exam, colposcopy, cone biopsy, endocervical curettage, X-ray, CT scan, cystoscopy and proctoscopy.

### Therapeutic Methods and Administration

An IGF1R inhibitor can be used to inhibit or reduce the growth or proliferation of any a malignant cell or treat a medical condition mediated by IGF1R. Such treatment or inhibition or reduction of growth or proliferation of a cell, in a subject's body, can be achieved by administering a therapeutically effective dosage of the IGF1R inhibitor which is adjustable or alterable according to observations relating to IGFBP2 levels in the patient's body (e.g., as discussed herein). In an embodiment of the invention, any tumor associated with or known to be associated with IGF1R expression and with elevated IGFBP2 levels, e.g., as per knowledge commonly held in the art, for example, as expressed in scientific literature, is suitable for treatment with an IGF1R inhibitor, e.g., as discussed herein.

IGFBP2 may, in an embodiment of the invention, serve as a marker for efficacy of an IGF1R inhibitor. An embodiment of the invention includes a method for assessing whether an IGF1R inhibitor inhibits growth or survival of a tumor in a patient being treated for said tumor by being administered said IGF1R inhibitor; or for assessing efficacy of said inhibitor in said patient comprising: determining IGFBP2 levels in the patient over time; wherein said tumor growth or survival is determined to be inhibited or said inhibitor is determined to be efficacious if said IGFBP2 levels decrease or remain unchanged over time during said treatment and wherein said tumor growth or survival is determined not to be inhibited or said inhibitor is determined not to be efficacious if said IGFBP2 levels increase over time.

In an embodiment of the invention, a cancer or other medical condition which is treatable with an IGF1R inhibitor using the methods of the present invention includes osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

The term “patient” or “subject” includes any organism, preferably an animal, more preferably a mammal (e.g., rat, mouse, dog, cat, rabbit) and most preferably a human.

As stated above, in an embodiment of the invention, where possible, an IGF1R inhibitor is administered to a subject in accordance with the Physicians' Desk Reference 2003 (Thomson Healthcare; 57th edition (Nov. 1, 2002)) or as set forth herein.

An IGF1R inhibitor can be administered by an invasive route such as by injection. Administration by a non-invasive route (e.g., orally; for example, in a pill, capsule or tablet) is also within the scope of the present invention. In an embodiment of the invention, an anti-IGF1R antibody (e.g., 15H12/19D12 LCF/HCA), or pharmaceutical composition thereof, is administered intravenously, subcutaneously, intramuscularly, intraarterially or intratumorally.

An IGF1R inhibitor can be administered with medical devices known in the art. For example, a pharmaceutical composition of the invention can be administered by injection with a hypodermic needle.

The pharmaceutical compositions of the invention may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

Examples of well-known implants and modules for administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

The present invention relates to methods for treating medical conditions mediated by expression or activity of IGF1R. Expression of IGF1R by a patient's tumor cells can be determined using conventional techniques commonly held in the art. For example, IGF1R expression can be identified by western blot analysis (e.g., of biopsied tumor cells) using any of several anti-IGF1R antibodies which are commercially available (e.g., N-20, C-20 or H-60 from Santa Cruz Biotechnology; Santa Cruz, Calif.; alpha IR-3 from Oncogene Research/Calbiochem; San Diego, Calif.). Alternatively, certain cancers are simply known by practitioners of ordinary skill in the art to express IGF1R. Expression of IGFBP2 can be assayed e.g., as set forth above.

### Pharmaceutical Compositions

In an embodiment of the invention, an IGF1R inhibitor is incorporated into a pharmaceutical composition, along with a pharmaceutically acceptable carrier, suitable for administration to a subject in vivo. The scope of the present invention includes pharmaceutical compositions which are suitable to be administered to a subject by any route (parenteral or non-parenteral) including, for example, oral, ocular, topical, pulmonary (inhalation), intratumoral injection, intravenous injection, subcutaneous injection or intramuscular injection.

For general information concerning formulations, see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, N.Y.; Lieberman, et al., (eds.) (1990) Pharmaceutical Dosage Forms: Tablets Dekker, N.Y.; and Lieberman, et al., (eds.) (1990), Pharmaceutical Dosage Forms: Disperse Systems Dekker, N.Y., Kenneth A. Walters (ed.) (2002) Dermatological and Transdermal Formulations (Drugs and the Pharmaceutical Sciences), Vol 119, Marcel Dekker.

In an embodiment of the invention, wherein the IGF1R inhibitor is an anti-IGF1R antibody (e.g., mature 15H12/19D12 LCB/HCB, LCC/HCB, LCF/HCA or LCD/HCA), the pharmaceutical composition comprises sodium acetate trihydrate (e.g., USP) at 2.30 g/l; glacial acetic acid (e.g., USP/Ph. Eur.) at 0.18 g/l; sucrose extra pure (e.g., NF, Ph. Eur, BP) at 70.0 g/l; the antibody at any concentration such as 20.0 g/l and water for injection (e.g., USP/Ph. Eur); pH about 5.5. In an embodiment of the invention, the composition is lyophilized/dessicated (lacking the water component) and is reconstituted (by adding water) at a point prior to use.

Pharmaceutically acceptable carriers are conventional and very well known in the art. Examples include aqueous and nonaqueous carriers, stabilizers, antioxidants, solvents, dispersion media, coatings, antimicrobial agents, buffers, serum proteins, isotonic and absorption delaying agents, and the like that are physiologically compatible. In an embodiment of the invention, the carrier is suitable for injection into a subject's body.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Examples of pharmaceutically-acceptable antioxidants include: water soluble antioxidants such as ascorbic acid, cysteine hydrochloride, sodium bisulfate, sodium metabisulfite, sodium sulfite and the like; and oil-soluble antioxidants such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Prevention of the presence of microorganisms may be ensured both by sterilization procedures, and by the inclusion of various antimicrobial agents such as EDTA, EGTA, paraben, chlorobutanol, phenol sorbic acid, and the like.

Suitable buffers which may be included in the pharmaceutical compositions of the invention include L-histidine-based buffers, phosphate-based buffers (e.g., phosphate buffered saline, pH≅7), sorbate-based buffers or glycine-based buffers.

Serum proteins which may be included in the pharmaceutical compositions of the invention include, in an embodiment of the invention, human serum albumin.

Isotonic agents, such as sugars (e.g., sucrose), ethanol, polyalcohols (e.g., glycerol, propylene glycol, liquid polyethylene glycol, mannitol or sorbitol), sodium citrate or sodium chloride (e.g., buffered saline) may also be included in the pharmaceutical compositions of the invention. In an embodiment of the invention, the sugar, for example, glucose or sucrose is present at a high concentration (e.g., about 10-100 mg/ml, e.g., 50 mg/ml, 60 mg/ml or 70 mg/ml).

Prolonged absorption of an injectable pharmaceutical form may be brought about by the inclusion of agents which delay absorption such as aluminum monostearate and/or gelatin.

Dispersions can also be prepared in glycerol, liquid polyethylene glycols, and mixtures thereof and in oils.

Pharmaceutically acceptable carriers include sterile aqueous solutions or dispersions and sterile powders for the extemporaneous preparation of sterile injectable solutions or dispersions. The use of such media and agents for pharmaceutically active substances is well known in the art.

Sterile injectable solutions comprising an anti-IGF1R antibody can be prepared by incorporating the antibody or antigen-binding fragment thereof in the required amount in an appropriate solvent, optionally with one or a combination of ingredients enumerated above, as required, followed by sterilization microfiltration. Generally, dispersions are prepared by incorporating the antibody into a sterile vehicle that contains a basic dispersion medium and the required other ingredients from those enumerated above. In the case of sterile powders for the preparation of sterile injectable solutions, possible methods of preparation are vacuum drying and freeze-drying (lyophilization) that yield a powder of the active ingredient plus any additional, desired ingredients therein.

In an embodiment of the invention, an anti-IGF1R antibody of the invention is in a pharmaceutical formulation comprising a therapeutically effective amount of said antibody, a buffer and sucrose. For example, in an embodiment of the invention, the buffer is any one of phosphate buffer, citrate buffer, histidine buffer, glycine buffer or acetate buffer. The pharmaceutical formulation can be within any suitable pH range. In an embodiment of the invention, the pH is about 5.0, 5.5, 6.0, 7.5, or between about 5.5 and about 6 or between about 5 and about 7.

An IGF1R inhibitor including an anti-IGF1R antibody or antigen-binding fragment thereof can, in an embodiment of the invention, be orally administered. Pharmaceutical compositions for oral administration may contain, in addition to the antibody or antigen-binding fragment thereof, additives such as starch (e.g., potato, maize or wheat starch or cellulose), starch derivatives (e.g., microcrystalline cellulose or silica), sugars (e.g., lactose), talc, stearate, magnesium carbonate or calcium phosphate. In order to ensure that oral compositions comprising an antibody or antigen-binding fragment of the invention are well tolerated by the patient's digestive system, mucus formers or resins may be included. It may also be desirable to improve tolerance by formulating the antibody or antigen-binding fragment in a capsule which is insoluble in the gastric juices. An exemplary pharmaceutical composition of this invention in the form of a capsule is prepared by filling a standard two-piece hard gelatin capsule with the antibody or antigen-binding fragment of the invention in powdered form, lactose, talc and magnesium stearate. Oral administration of immunoglobulins has been described (Foster, et al., (2001) Cochrane Database System rev. 3:CD001816)

An IGF1R inhibitor may also, in an embodiment of the invention, be administered by inhalation. A suitable pharmaceutical composition for inhalation may be an aerosol. An exemplary pharmaceutical composition for inhalation of an antibody or antigen-binding fragment of the invention can include: an aerosol container with a capacity of 15-20 ml comprising the antibody or antigen-binding fragment of the invention, a lubricating agent, such as polysorbate 85 or oleic acid, dispersed in a propellant, such as freon, preferably in a combination of 1,2-dichlorotetrafluoroethane and difluorochloromethane. In an embodiment of the invention, the composition is in an appropriate aerosol container adapted for either intranasal or oral inhalation administration.

### Kits and Articles of Manufacture

Kits and articles of manufacture of the present invention include an IGF1R inhibitor, combined, in an embodiment of the invention, with a pharmaceutically acceptable carrier, in a pharmaceutical formulation, for example in a pharmaceutical dosage form such as a pill, a powder, an injectable liquid or reconstitutable powder thereof, a tablet, dispersible granules, a capsule, a cachet or a suppository. See for example, Gilman et al. (eds.) (1990), The Pharmacological Bases of Therapeutics, 8th Ed., Pergamon Press; and Remington's Pharmaceutical Sciences, supra, Easton, Pa.; Avis et al. (eds.) (1993) Pharmaceutical Dosage Forms: Parenteral Medications Dekker, N.Y.; Lieberman et al. (eds.) (1990) Pharmaceutical Dosage Forms: Tablets Dekker, N.Y.; and Lieberman et al. (eds.) (1990), Pharmaceutical Dosage Forms: Disperse Systems Dekker, N.Y.

The kits and articles of manufacture of the present invention also include information, for example in the form of a package insert or label, indicating that the target of the IGF1R inhibitory agent is IGF1R and that cancer patients (e.g., patients with a tumor expressing IGF1R) exhibiting elevated levels of IGFBP2 are likely to be responsive to an IGF1R inhibitor e.g, as discussed herein. In an embodiment of the invention, the label indicates that efficacy of the IGF1R inhibitor in a patient can be evaluated by monitoring IGFBP2 levels in the patient as set forth herein. Furthermore, in an embodiment of the invention, the label indicates that dosage of the IGF1R inhibitor can be evaluated by the methods discussed herein or that the effect of the inhibitor on IGF1R or any member of the IGF1R pathway can be evaluated by the methods discussed herein.

The insert or label may take any form, such as paper or on electronic media such as a magnetically recorded medium (e.g., floppy disk) or a CD-ROM.

The label or insert may also include other information concerning the pharmaceutical compositions and dosage forms in the kit or article of manufacture. Generally, such information aids patients and physicians in using the enclosed pharmaceutical compositions and dosage forms effectively and safely. For example, the following information regarding the IGF1R inhibitory agent may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references and patent information.

The present invention further comprises a method for manufacturing an IGF1R inhibitor or a pharmaceutical composition thereof comprising a pharmaceutically acceptable carrier said method comprising combining, in a package, the inhibitor or composition; and a label conveying that the inhibitor or composition dosage or the inhibitor's or composition's inhibition of IGF1R or any member of the IGF1R pathway may be evaluated using any of the methods discussed herein.

### EXAMPLES

This section is intended to further describe the present invention and should not be construed to further limit the invention. Any composition or method set forth herein constitutes part of the present invention.

### Example 1

### Treatment of Anti-IGF1R Mab 19D12 Decreased IGFBP2 Level in Xenograft Tumors

This example demonstrated that anti-IGF1R (comprising mature polypeptide Ig chains of the amino acid sequence of SEQ ID NOs: 8 and 10) decreased the level of IGFBP2 per microgram of total tumor protein in neuroblastoma tumor models.

Athymic nude mice were inoculated with SK-N-MC or SK-N-AS (human neuroblastoma) tumor cells in the right flank, subcutaneously, along with Matrigel (1:1 cells:gel). In these experiments, 5×10⁶ cells/mouse in a 1:1 mix with regular matrigel were inoculated subcutaneously. Tumor size was measured with calipers and the data was entered into the labcat program. Mice were grouped with an average tumor size of 100 mm³. Mice were dosed twice per week, intraperitoneally (i.p.) with antibody 19D12. Tumor size and mouse body weight was measured twice weekly after treatment.

Tumors were dissected out at the end of the studies, snap frozen, and stored at −80° C. until analysis. Frozen xenograft tumor tissues were homogenized and lysed in buffer containing 50 mM Hepes, pH 7.4, 150 mM NaCl, 10% glycerol, 1% Triton X-100, 1.5 mM MgCl₂, 2 mM Na₃VO₄ and protease inhibitor cocktail (Complete™, Roche). Samples were spun for 13,000 rpm for 10 minutes at 4° C. after incubation on ice for 30 minutes. Supernatants were collected and protein concentrations of the lysates were determined by Bio-Rad assay.

TABLE 3
IGFBP2 level in SK-N-MC xenograft tumors

| | Average | Tumor | |
|---|---|---|---|
| Treatment (n = 10) | IGFBP2 Level (pg/ug)* | volume (mm³) | SD |
| IgG1 Control | 4.66 | 665 | 0.78 |
| 0.004 mg 19D12/IgG1 | 1.99 | 331 | 0.48 |
| 0.1 mg 19D12/IgG1 | 1.98 | 344 | 0.34 |
| 0.5 mg 19D12/IgG1 | 1.79 | 335 | 0.47 |

TABLE 4
IGFBP2 level in SK-N-AS xenograft tumors

| | Average | Tumor | |
|---|---|---|---|
| Treatment (n = 5) | IGFBP2 Level (pg/ug)* | volume (mm³) | SD |
| IgG1 Control | 4.57 | 1735 | 2.36 |
| 0.5 mg 19D12/IgG1 | 0.40 | 327 | 0.21 |

*picograms of IGFBP2 per microgram of total tumor protein.

The present invention relates to methods for evaluating an IGF1R inhibitor regimen (e.g., the dosage of the inhibitor), administered to a subject, by observing IGFBP2 levels in the subject. Decreasing IGFBP2 levels in the tumor correlate with inhibition of the IGF1R pathway as well as with inhibition of the downstream effects of the pathway, e.g., tumor growth. The decrease of IGFBP2 in the tumor tissue should reflect a decrease in IGFBP2 in the blood of the subject receiving the IGF1R inhibitor since IGFBP2 is a secreted protein. The data in this example support this point.

### Example 2

### Treatment of Anti-IGF1R Mab 19D12 Decreased Serum IGFBP2 Level in Monkeys

This example demonstrates that IGFBP2 levels drop in monkeys receiving an anti-IGF1R antibody.

Dosing. Monkeys in group C1 were dosed with vehicle control (placebo) once weekly for 13 weeks starting at Day 0. Monkeys in group T1 were dosed with anti-IGF1R Mab 19D12 (comprising mature polypeptide Ig chains of the amino acid sequence of SEQ ID NOs: 8 and 10) once weekly at 10 mg/mg for 13 weeks starting at Day 0.

Blood samples were collected via the femoral artery/vein at indicated time points into a serum separator tube and centrifuged to obtain the serum. Serum samples were stored at −80° C. until analysis.

Measurement of IGFBP2. The R&D Duoset Human IGFBP2 ELISA Development System was chosen and the following protocol was used: Plates were coated with 100 ul of 2 ug/ml anti-Hu IGFBP2 overnight at 4° C. After each step, plates were rinsed 4×250 ul of wash buffer. 100 ul of block buffer was added for 1 hour. And a rinse was performed. Standards and diluted samples were added and incubated 2 hours at room temperature on a shaker. A rinse was performed. 100 ul of 100 ng/ml of secondary anti-IGFBP2 antibody as the detection antibody was added for 2 hours with shaking. A rinse was performed. Added 100 ul Streptavidin-HRP for 20 minutes with shaking. A rinse was performed. Added 100 ul of a 1:1 mix of substrate solution for 20 minutes with shaking. Added 50 ul of stop solution. Tapped plate to mix thoroughly. Read at 450 nM. The standard curve was reduced with a 4 parameter curve fit with SOFTmax Pro software.

The results of the foregoing experiments were as follows:

TABLE 5
IGFBP2 levels observed in monkeys dosed with
placebo or with anti-IGF1R antibody.

| | | Dose | Time | IGFBP2 |
|---|---|---|---|---|
| Animal | SEX | Group | Point | (ng/ml) |
| 101 | M | C1 | WEEK -5 | 151 |
| 101 | M | C1 | WEEK -3 | 153 |
| 101 | M | C1 | Day 1 | 169 |
| 101 | M | C1 | Day 4 | 154 |
| 101 | M | C1 | Day 7 | 148 |
| 101 | M | C1 | Day 91 | 176 |
| 102 | M | C1 | WEEK -5 | 151 |
| 102 | M | C1 | WEEK -3 | 170 |
| 102 | M | C1 | Day 1 | 195 |
| 102 | M | C1 | Day 4 | 154 |
| 102 | M | C1 | Day 7 | 187 |
| 102 | M | C1 | Day 91 | 158 |
| 103 | M | C1 | WEEK -5 | 74 |
| 103 | M | C1 | WEEK -3 | 90 |
| 103 | M | C1 | Day 1 | 89 |
| 103 | M | C1 | Day 4 | 88 |
| 103 | M | C1 | Day 7 | 96 |
| 103 | M | C1 | Day 91 | 116 |
| 104 | M | C1 | WEEK -5 | 165 |
| 104 | M | C1 | WEEK -3 | 199 |
| 104 | M | C1 | Day 1 | 184 |
| 104 | M | C1 | Day 4 | 170 |
| 104 | M | C1 | Day 7 | 168 |
| 104 | M | C1 | Day 91 | 191 |
| 105 | M | C1 | WEEK -5 | 77 |
| 105 | M | C1 | WEEK -3 | 90 |
| 105 | M | C1 | Day 1 | 84 |
| 105 | M | C1 | Day 4 | 95 |
| 105 | M | C1 | Day 7 | 76 |
| 105 | M | C1 | Day 91 | 89 |
| 106 | M | C1 | WEEK -5 | 120 |
| 106 | M | C1 | WEEK -3 | 142 |
| 106 | M | C1 | Day 1 | 135 |
| 106 | M | C1 | Day 4 | 114 |
| 106 | M | C1 | Day 7 | 128 |
| 106 | M | C1 | Day 91 | 148 |
| 501 | F | C1 | WEEK -5 | 93 |
| 501 | F | C1 | WEEK -3 | 119 |
| 501 | F | C1 | Day 1 | 154 |
| 501 | F | C1 | Day 4 | 131 |
| 501 | F | C1 | Day 7 | 117 |
| 501 | F | C1 | Day 91 | 145 |
| 502 | F | C1 | WEEK -5 | 134 |
| 502 | F | C1 | WEEK -3 | 211 |
| 502 | F | C1 | Day 1 | 205 |
| 502 | F | C1 | Day 4 | 161 |
| 502 | F | C1 | Day 7 | 174 |
| 502 | F | C1 | Day 91 | 227 |
| 503 | F | C1 | WEEK -5 | 130 |
| 503 | F | C1 | WEEK -3 | 158 |
| 503 | F | C1 | Day 1 | 132 |
| 503 | F | C1 | Day 4 | 154 |
| 503 | F | C1 | Day 7 | 136 |
| 503 | F | C1 | Day 91 | 150 |
| 504 | F | C1 | WEEK -5 | 75 |
| 504 | F | C1 | WEEK -3 | 66 |
| 504 | F | C1 | Day 1 | 65 |
| 504 | F | C1 | Day 4 | 62 |
| 504 | F | C1 | Day 7 | 65 |
| 504 | F | C1 | Day 91 | 56 |
| 505 | F | C1 | WEEK -5 | 64 |
| 505 | F | C1 | WEEK -3 | 68 |
| 505 | F | C1 | Day 1 | 87 |
| 505 | F | C1 | Day 4 | 70 |
| 505 | F | C1 | Day 7 | 69 |
| 505 | F | C1 | Day 91 | 66 |
| 506 | F | C1 | WEEK -5 | 85 |
| 506 | F | C1 | WEEK -3 | 86 |
| 506 | F | C1 | Day 1 | 93 |
| 506 | F | C1 | Day 4 | 81 |
| 506 | F | C1 | Day 7 | 79 |
| 506 | F | C1 | Day 91 | 78 |
| 1001 | M | T1 | WEEK -5 | 166 |
| 1001 | M | T1 | WEEK -3 | 173 |
| 1001 | M | T1 | Day 1 | 169 |
| 1001 | M | T1 | Day 4 | 92 |
| 1001 | M | T1 | Day 7 | 72 |
| 1001 | M | T1 | Day 91 | 78 |
| 1002 | M | T1 | WEEK -5 | 147 |
| 1002 | M | T1 | WEEK -3 | 136 |
| 1002 | M | T1 | Day 1 | 146 |
| 1002 | M | T1 | Day 4 | 99 |
| 1002 | M | T1 | Day 7 | 86 |
| 1002 | M | T1 | Day 91 | 79 |
| 1003 | M | T1 | WEEK -5 | 121 |
| 1003 | M | T1 | WEEK -3 | 160 |
| 1003 | M | T1 | Day 1 | 114 |
| 1003 | M | T1 | Day 4 | 88 |
| 1003 | M | T1 | Day 7 | 69 |
| 1003 | M | T1 | Day 91 | 80 |
| 1004 | M | T1 | WEEK -5 | 156 |
| 1004 | M | T1 | WEEK -3 | 188 |
| 1004 | M | T1 | Day 1 | 184 |
| 1004 | M | T1 | Day 4 | 163 |
| 1004 | M | T1 | Day 7 | 158 |
| 1004 | M | T1 | Day 91 | 144 |
| 1005 | M | T1 | WEEK -5 | 141 |
| 1005 | M | T1 | WEEK -3 | 163 |
| 1005 | M | T1 | Day 1 | 175 |
| 1005 | M | T1 | Day 4 | 126 |
| 1005 | M | T1 | Day 7 | 121 |
| 1005 | M | T1 | Day 91 | 170 |
| 1006 | M | T1 | WEEK -5 | 110 |
| 1006 | M | T1 | WEEK -3 | 114 |
| 1006 | M | T1 | Day 1 | 113 |
| 1006 | M | T1 | Day 4 | 80 |
| 1006 | M | T1 | Day 7 | 80 |
| 1006 | M. | T1 | Day 91 | 95 |
| 1501 | F | T1 | WEEK -5 | 124 |
| 1501 | F | T1 | WEEK -3 | 127 |
| 1501 | F | T1 | Day 1 | 138 |
| 1501 | F | T1 | Day 4 | 114 |
| 1501 | F | T1 | Day 7 | 94 |
| 1501 | F | T1 | Day 91 | 82 |
| 1502 | F | T1 | WEEK -5 | 141 |
| 1502 | F | T1 | WEEK -3 | 138 |
| 1502 | F | T1 | Day 1 | 145 |
| 1502 | F | T1 | Day 4 | 92 |
| 1502 | F | T1 | Day 7 | 72 |
| 1502 | F | T1 | Day 91 | 85 |
| 1503 | F | T1 | WEEK -5 | 281 |
| 1503 | F | T1 | WEEK -3 | 316 |
| 1503 | F | T1 | Day 1 | 253 |
| 1503 | F | T1 | Day 4 | 274 |
| 1503 | F | T1 | Day 7 | 296 |
| 1503 | F | T1 | Day 91 | 274 |
| 1504 | F | T1 | WEEK -5 | 100 |
| 1504 | F | T1 | WEEK -3 | 106 |
| 1504 | F | T1 | Day 1 | 89 |
| 1504 | F | T1 | Day 4 | 65 |
| 1504 | F | T1 | Day 7 | 62 |
| 1504 | F | T1 | Day 91 | 63 |
| 1505 | F | T1 | WEEK -5 | 70 |
| 1505 | F | T1 | WEEK -3 | 70 |
| 1505 | F | T1 | Day 1 | 60 |
| 1505 | F | T1 | Day 4 | 44 |
| 1505 | F | T1 | Day 7 | 43 |
| 1505 | F | T1 | Day 91 | 58 |
| 1506 | F | T1 | WEEK -5 | 96 |
| 1506 | F | T1 | WEEK -3 | 110 |
| 1506 | F | T1 | Day 1 | 107 |
| 1506 | F | T1 | Day 4 | 92 |
| 1506 | F | T1 | Day 7 | 70 |
| 1506 | F | T1 | Day 91 | 55 |

The C1 group received a placebo and the T1 group received the antibody.

### Example 3

### Treatment of Anti-IGF1R Mab 19D12 Decreases Serum IGFBP2 Level in Health Human Subjects

This example demonstrates that IGFBP2 levels decrease in response to IGF1R inhibition with an anti-IGF1R antibody.

Following an initial sampling of blood for the determination of baseline, untreated IGFBP2 levels, health human subjects were given a single intravenous infusion of anti-IGF1 R antibody (comprising mature polypeptide Ig chains of the amino acid sequence of SEQ ID NOs: 8 and 10) at 0.3 mg/kg, 1.0 mg/kg, 3.0 mg/kg, 10.0 mg/kg, and 20.0 mg/kg for 60 minutes. After treatment, blood was taken for analysis of IGFBP2 at day 1 (before the dose) and at 3, 6, 8, 10, 15 and 57 (“endpoint”) days post-dose. On days 15, 16 and 17, all subjects were also injected with recombinant human IGF-1 (subcutaneously, BID). The data gathered from these experiments is set forth below in Table 6.

TABLE 6
Summary of IGFBP2 levels observed in healthy human subjects
administered the indicated doses of anti-IGF1R antibody.

| Dose Group* | Time Points Post Dose | IGFBP2(ng/ml) | SD |
|---|---|---|---|
| 0.3 mg/kg | Baseline | 241.9 | 129.6 |
| | Day 3 | 169.4 | 83.5 |
| | Day6 | 153 | 103.9 |
| | Day8 | 157 | 127.9 |
| | Day10 | 200 | 172.5 |
| | Day15 | 292 | 177.5 |
| | Endpoint | 520 | 326.9 |
| 1.0 mg/kg | Baseline | 268 | 119.8 |
| | Day 3 | 180 | 97.7 |
| | Day6 | 154 | 126.3 |
| | Day8 | 125 | 85.8 |
| | Day10 | 170 | 170.6 |
| | Day15 | 180 | 147.9 |
| | Endpoint | 279 | 196.2 |
| 3.0 mg/kg | Baseline | 237 | 122.7 |
| | Day 3 | 154 | 58.9 |
| | Day6 | 119 | 54.3 |
| | Day8 | 116 | 55.5 |
| | Day10 | 99 | 50.0 |
| | Day15 | 109 | 41.9 |
| | Endpoint | 183 | 110.0 |
| 10 mg/kg | Baseline | 247 | 60.1 |
| | Day 3 | 156 | 51.2 |
| | Day6 | 123 | 53.2 |
| | Day8 | 129 | 49.4 |
| | Day10 | 118 | 68.7 |
| | Day15 | 136 | 89.9 |
| | Endpoint | 211 | 172.0 |
| 20 mg/kg | Baseline | 190 | 100.1 |
| | Day 3 | 129 | 45.2 |
| | Day6 | 84 | 30.6 |
| | Day8 | 83 | 32.2 |
| | Day10 | 99 | 38.6 |
| | Day15 | 97 | 18.9 |
| | Endpoint | 139 | 64.1 |

The IGFBP2 values are the mean levels observed in 8 subjects, in each dose group, which included both 6 subjects dosed with the antibody and 2 subjects dosed with a placebo.
Saturation of the IGF1 receptors in a subject receiving the anti-IGF1R antibody correlates with a reduction of IGFBP2 levels by at least 51% of the baseline IGFBP2 level.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

We claim:

1. A method for monitoring the effect of an IGF1R inhibitor on IGF1R receptor in the body of a subject administered said inhibitor comprising evaluating IGFBP2 levels in the body of the subject over time.

2. The method of claim 1 wherein the inhibitor is determined to inhibit the receptor if IGFBP2 levels are observed to decrease over time following said administration; or wherein the inhibitor is determined not to inhibit the receptor if IGFBP2 levels are not observed to decrease over time following said administration.

3. The method of claim 1 wherein the subject suffers from a medical condition mediated by IGF1R activity or expression and wherein the inhibitor is determined to inhibit the receptor sufficiently if IGFBP2 levels are observed to decrease by at least 51% over time following a first administration of said inhibitor; or wherein the inhibitor is determined not to inhibit the receptor sufficiently if IGFBP2 levels are not observed to decrease by at least 51% over time following a first administration of said inhibitor.

4. The method of claim 1 comprising:
(i) measuring an IGFBP2 level in the body of said subject;
(ii) administering one or more doses of said inhibitor to said subject;
(iii) measuring an IGFBP2 level in the body of said subject following said administration;
(iv) comparing the level of IGFBP2 measured in step (i) with the level of IGFBP2 measured in step (iii);
wherein the inhibitor is determined to inhibit the receptor if IGFBP2 levels are observed to decrease over time following said administration; or wherein the inhibitor is determined not to inhibit the receptor if IGFBP2 levels are not observed to decrease over time following said administration.

5. The method of claim 1 wherein the IGF1R inhibitor is an antibody or antigen-binding fragment thereof that binds specifically to IGF1R.

6. The method of claim 5 wherein the antibody or fragment comprises one or more complementarity determining regions (CDRs) selected from the group consisting of:

| | |
|---|---|
| RASQSIGSSLH; | (SEQ ID NO:99) |
| YASQSLS; | (SEQ ID NO:100) |
| HQSSRLPHT; | (SEQ ID NO:101) |
| SFAMH | (SEQ ID NO:102) |
| GFTFSSFAMH; | (SEQ ID NO:107) |
| VIDTRGATYYADSVKG; | (SEQ ID NO:103) |
| and | |
| LGNFYYGMDV; | (SEQ ID NO:104) |

or a mature fragment of a light chain immunoglobulin which comprises the amino acid sequence of SEQ ID NO: 2, 4, 6 or 8;
or a mature fragment of a heavy chain immunoglobulin which comprises the amino acid sequence of SEQ ID NO: 10 or 12; or a pharmaceutical composition thereof which comprises a pharmaceutically acceptable carrier.

7. The method of claim 1 wherein the subject suffers from a medical disorder mediated by IGF1R expression or activity.

8. The method of claim 7 wherein the disorder is a member selected from the group consisting of: osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

9. The method of claim 1 wherein the subject is also administered one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL 13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 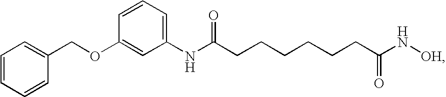
CG-781 CG-1521, 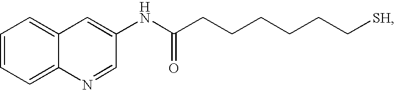
SB-556629, chlamydocin, JNJ-16241199, 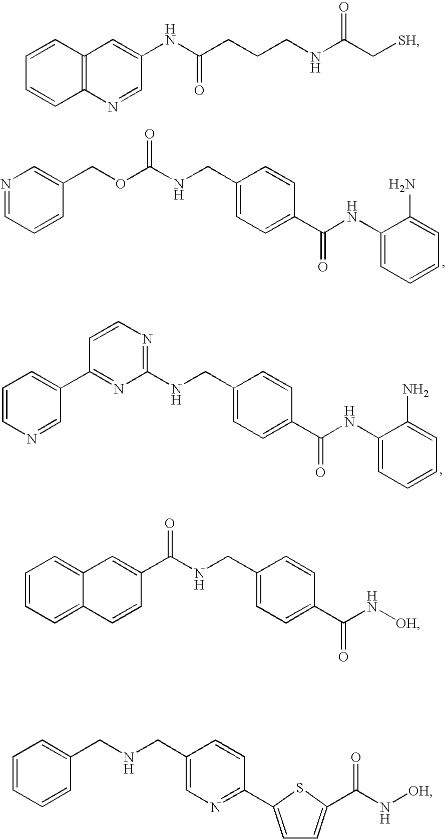
vorinostat, etoposide, gemoitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258, 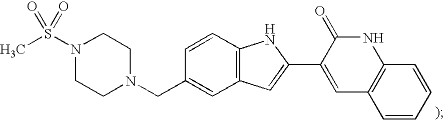
3-[5-(methylsulfonylpiperadinemethyl)-indolyl]-quinolone, vatalanib, AG-013736, AVE-0005, the acetate salt of [D-Ser(Bu t) 6, Azgly 10](pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 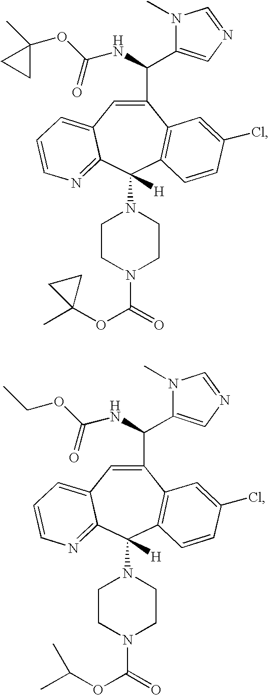
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK1 86619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

10. The method of claim 1 wherein the IGFBP2 level is determined using a radioimmunoassay (RIA), a western blot or an enzyme linked immunosorbent assay (ELISA) of a sample from the subject.

11. The method of claim 1 wherein IGFBP2 is measured in blood, serum or plasma from the subject.

12. A method for evaluating dosage of an IGF1R inhibitor administered to a subject with a medical condition mediated by IGF1R expression or activity comprising administering a dose of said inhibitor to said subject and evaluating IGFBP2 levels in the body of the subject; wherein said dosage is determined to be insufficient if IGFBP2 levels are not observed to decrease by at least 51% of an IGFBP2 level measured prior to first administration of said inhibitor following said administration; or wherein said dosage is determined to be sufficient if IGFBP2 levels are observed to decrease by at least 51% of an IGFBP2 level measured prior to first administration of said inhibitor following said administration.

13. The method of claim 12 comprising:
(i) measuring an IGFBP2 level in the body of said subject before any treatment with said inhibitor;
(ii) administering one or more doses of said inhibitor to said subject;
(iii) measuring an IGFBP2 level in the body of said subject following said administration;
(iv) comparing the level of IGFBP2 measured in step (i) with the level of IGFBP2 measured in step (iii);
wherein said dosage is determined to be insufficient if IGFBP2 levels are not observed to decrease by at least 51% over time following said administration; or wherein said dosage is determined to be sufficient if IGFBP2 levels are observed to decrease by at least 51% over time following said administration.

14. The method of claim 12 wherein the IGF1R inhibitor is an antibody or antigen-binding fragment thereof that binds specifically to IGF1R.

15. The method of claim 14 wherein the antibody or fragment comprises one or more complementarity determining regions (CDRs) selected from the group consisting of:

| | |
|---|---|
| RASQSIGSSLH; | (SEQ ID NO:99) |
| YASQSLS; | (SEQ ID NO:100) |
| HQSSRLPHT; | (SEQ ID NO:101) |
| SFAMH | (SEQ ID NO:102) |
| GFTFSSFAMH; | (SEQ ID NO:107) |
| VIDTRGATYYADSVKG; | (SEQ ID NO:103) |
| and | |
| LGNFYYGMDV; | (SEQ ID NO:104) |

or a mature fragment of a light chain immunoglobulin which comprises the amino acid sequence of SEQ ID NO: 2, 4, 6 or 8; or a mature fragment of a heavy chain immunoglobulin which comprises the amino acid sequence of SEQ ID NO: 10 or 12; or a pharmaceutical composition thereof which comprises a pharmaceutically acceptable carrier.

16. The method of claim 15 wherein the antibody or fragment is a monoclonal antibody.

17. The method of claim 12 wherein the disorder is a member selected from the group consisting of: osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

18. The method of claim 12 wherein the subject is also administered one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 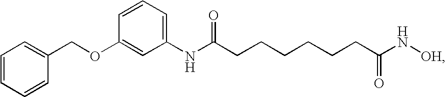
CG-781, CG-1521, 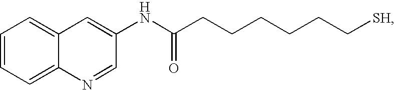
SB-556629, chlamydocin, JNJ-16241199, 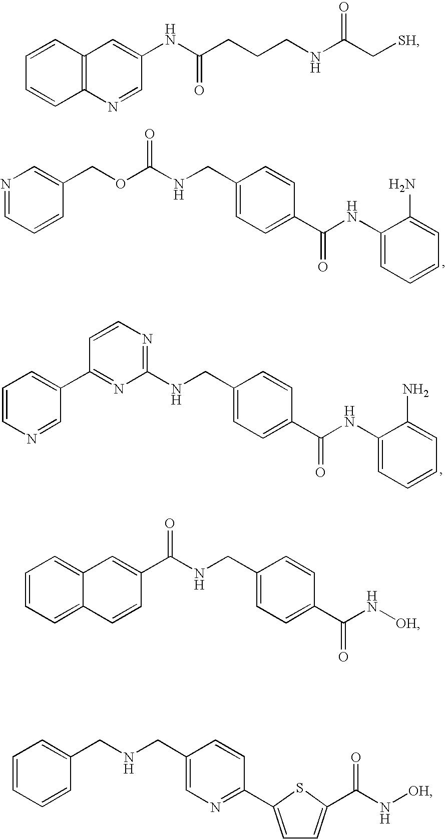
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258, 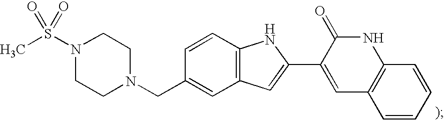
3-[5-(methylsulfonylpiperadinemethyl)-indolyl]-quinolone, vatalanib, AG-013736, AVE-0005, the acetate salt of [D-Ser(Bu t) 6, Azgly 10](pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 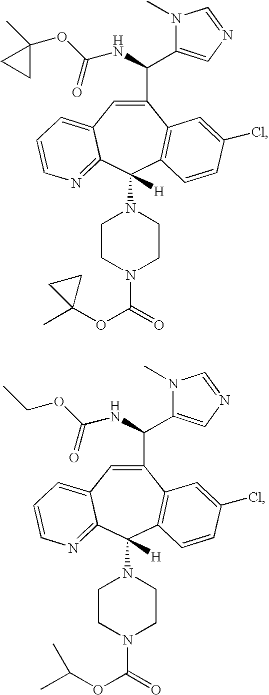
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

19. The method of claim 12 wherein the IGFBP2 level is determined using a radioimmunoassay (RIA), a western blot or an enzyme linked immunosorbent assay (ELISA) of a sample from the subject.

20. The method of claim 12 wherein IGFBP2 is measured in blood, serum or plasma from the subject.

21. A method for determining if a subject has a medical condition that is responsive to an IGF1R inhibitor comprising administering said inhibitor to said subject and evaluating IGFBP2 levels in the body of the subject over time;
wherein said condition is determined to be unresponsive to said inhibitor if the IGFBP2 levels are not observed to decrease over time following said administration; or wherein the condition is determined to be responsive to said inhibitor if the IGFBP2 levels are observed to decrease over time following said administration.

22. The method of claim 21 comprising:
(i) measuring an IGFBP2 level in the body of said subject;
(ii) administering one or more doses of said inhibitor to said subject;
(iii) measuring an IGFBP2 level in the body of said subject following said administration;
(iv) comparing the level of IGFBP2 measured in step (i) with the level of IGFBP2 measured in step (iii); wherein said condition is determined to be unresponsive to said inhibitor if the IGFBP2 levels are not observed to decrease over time following said administration; or wherein the condition is determined to be responsive to said inhibitor if the IGFBP2 levels are observed to decrease over time following said administration.

23. The method of claim 21 wherein the IGF1R inhibitor is an antibody or antigen-binding fragment thereof that binds specifically to IGF1R.

24. The method of claim 23 wherein the antibody or fragment comprises one or more complementarity determining regions (CDRs) selected from the group consisting of:

| | |
|---|---|
| RASQSIGSSLH; | (SEQ ID NO:99) |
| YASQSLS; | (SEQ ID NO:100) |
| HQSSRLPHT; | (SEQ ID NO:101) |
| SFAMH | (SEQ ID NO:102) |
| GFTFSSFAMH; | (SEQ ID NO:107) |
| VIDTRGATYYADSVKG; | (SEQ ID NO:103) |
| and | |
| LGNFYYGMDV; | (SEQ ID NO:104) |

or a mature fragment of a light chain immunoglobulin which comprises the amino acid sequence of SEQ ID NO: 2, 4, 6 or 8; or a mature fragment of a heavy chain immunoglobulin which comprises the amino acid sequence of SEQ ID NO: 10 or 12; or a pharmaceutical composition thereof which comprises a pharmaceutically acceptable carrier.

25. The method of claim 22 wherein step (i) is performed on a subject prior to any administration of said inhibitor.

26. The method of claim 21 wherein the disorder is a member selected from the group consisting of: osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

27. The method of claim 21 wherein the subject is also administered one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 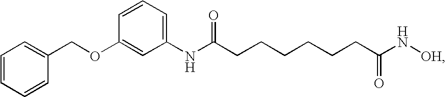
CG-781, CG-1521, 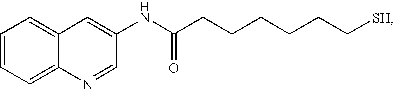
SB-556629, chlamydocin, JNJ-16241199, 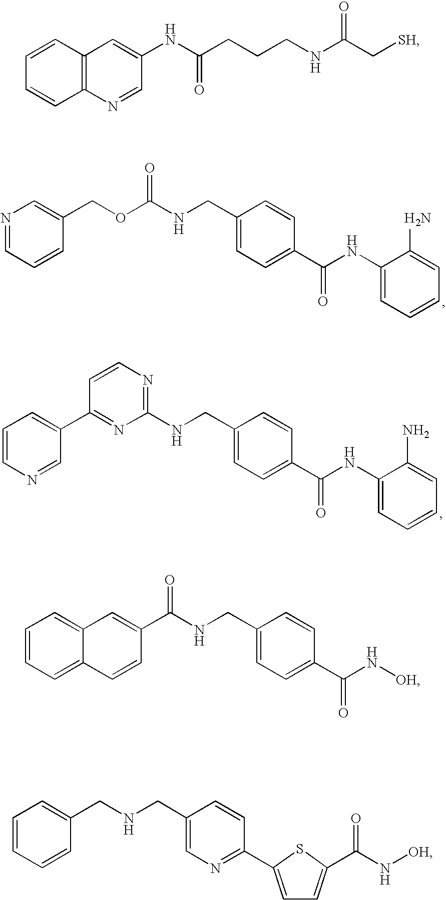
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258, 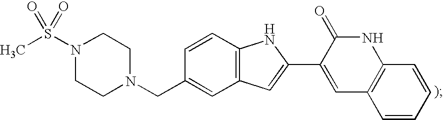
3-[5-(methylsulfonylpiperadinemethyl)-indolyl]-quinolone, vatalanib, AG-013736, AVE-0005, the acetate salt of [D-Ser(Bu t) 6, Azgly 10](pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 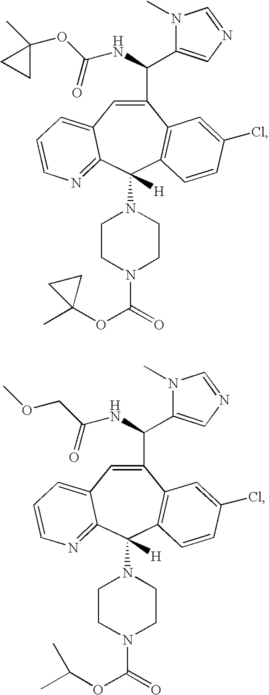
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BAS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

28. The method of claim 21 wherein the IGFBP2 level is determined using a radioimmunoassay (RIA), a western blot or an enzyme linked immunosorbent assay (ELISA) of a sample from the subject.

29. The method of claim 21 wherein IGFBP2 is determined in blood, serum or plasma from the subject.

30. A method for monitoring the effect of an IGF1R inhibitor on IGFBP2 concentration in the body of a subject administered said inhibitor comprising measuring IGFBP2 levels in the body of the subject over time during a course of treatment with said inhibitor.

31. The method of claim 30 comprising:
(i) measuring an IGFBP2 concentration in the body of said subject;
(ii) administering one or more doses of said inhibitor to said subject;
(iii) measuring an IGFBP2 concentration in the body of said subject following said administration;
(iv) comparing the level of IGFBP2 measured in step (i) with the level of IGFBP2 measured in step (iii); wherein the inhibitor is determined to lower the IGFBP2 concentration if the level measured in step (i) is higher than the concentration measured in step (iii); and wherein the inhibitor is determined not to lower the IGFBP2 concentration if the level measured in step (i) is not higher than the concentration measured in step (iii).

32. The method of claim 31 wherein step (i) is performed prior to any administration of said inhibitor.

33. A method for treating a medical condition, in a subject, mediated by IGF1R expression or activity comprising:
(i) measuring an IGFBP2 level in the body of said subject prior to any administration of an IGF1R inhibitor;
(ii) administering one or more doses of an IGF1R inhibitor to said subject;
(iii) measuring an IGFBP2 level in the body of said subject following said administration;
(iv) comparing the level of IGFBP2 measured in step (i) with the level of IGFBP2 measured in step (iii); and
(v) increasing dosage of said inhibitor if the IGFBP2 level does not decrease by at least 51% following said administration; or maintaining or decreasing dosage if the IGFBP2 level does decrease by at least 51% following said administration.

34. The method of claim 33 wherein the IGF1R inhibitor is an antibody or antigen-binding fragment thereof that binds specifically to IGF1R.

35. The method of claim 34 wherein the antibody or fragment comprises one or more complementarity determining regions (CDRs) selected from the group consisting of:

| | |
|---|---|
| RASQSIGSSLH; | (SEQ ID NO:99) |
| YASQSLS; | (SEQ ID NO:100) |
| HQSSRLPHT; | (SEQ ID NO:101) |
| SFAMH | (SEQ ID NO:102) |
| GFTFSSFAMH; | (SEQ ID NO:107) |
| VIDTRGATYYADSVKG; | (SEQ ID NO:103) |
| and | |
| LGNFYYGMDV; | (SEQ ID NO:104) |

or a mature fragment of a light chain immunoglobulin which comprises the amino acid sequence of SEQ ID NO: 2, 4, 6 or 8; or a mature fragment of a heavy chain immunoglobulin which comprises the amino acid sequence of SEQ ID NO: 10 or 12; or a pharmaceutical composition thereof which comprises a pharmaceutically acceptable carrier.

36. The method of claim 33 wherein the antibody or fragment is a monoclonal antibody.

37. The method of claim 33 wherein the disorder is a member selected from the group consisting of: osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

38. The method of claim 33 wherein the subject is also administered one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 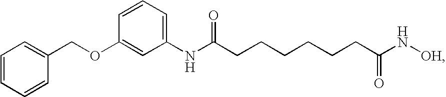
CG-781, CG-1521, 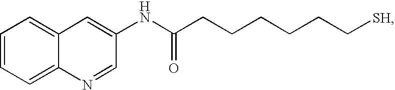
SB-556629, chlamydocin, JNJ-16241199, 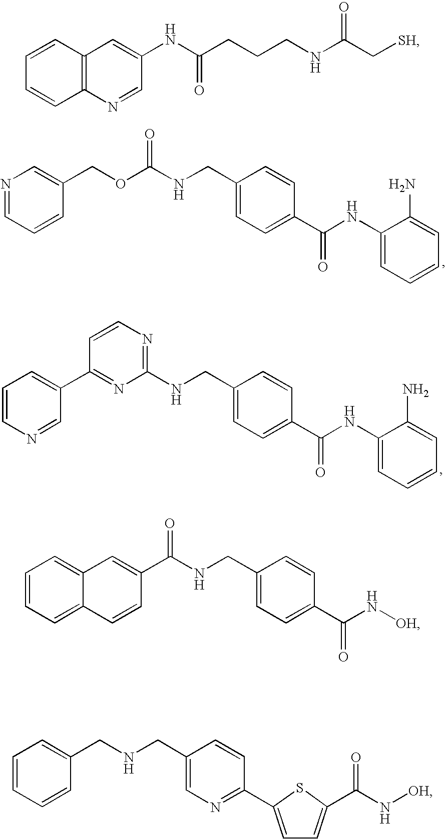
vorinostat, etoposide, gemcitabine, doxorubicin, liposomal doxorubicin, 5′-deoxy-5fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258, 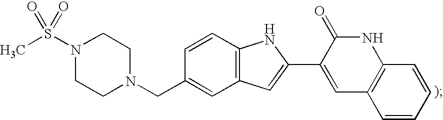
3-[5-(methylsulfonylpiperadinemethyl)-indolyl]-quinolone, vatalanib, AG-013736, AVE-0005, the acetate salt of [D-Ser(Bu t) 6, Azgly 10](pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 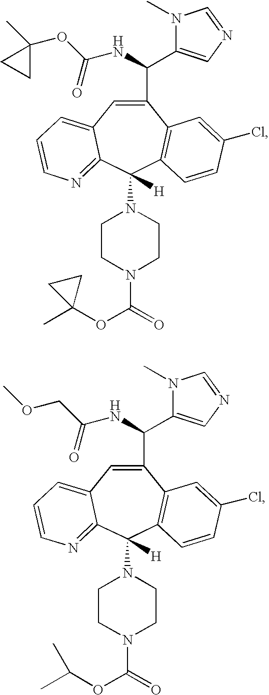
BMS-214662, tipifarnib, amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine dactinomycin, daunorubicin, diethylstilbestrol, epirubicin, fludarabine, fludrocortisone, fluoxymesterone, flutamide, hydroxyurea, idarubicin, ifosfamide, imatinib, leucovorin leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-1 2, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

39. A method for selecting a dose of an IGF1R inhibitor comprising administering a dose of said inhibitor to a subject with a medical condition mediated by IGF1R expression or activity and evaluating IGFBP2 levels in the body of the subject; wherein said dosage is selected if IGFBP2 levels are observed to decrease, following said administration, by at least 51% of an IGFBP2 level measured prior to first administration of said inhibitor.

40. The method of claim 39 comprising:
(i) measuring an IGFBP2 level in the body of said subject before first treatment with said inhibitor;
(ii) administering one or more doses of said inhibitor to said subject;
(iii) measuring an IGFBP2 level in the body of said subject following said administration;
(iv) comparing the level of IGFBP2 measured in step (i) with the level of IGFBP2 measured in step (iii);
wherein said dose is selected if IGFBP2 levels are observed to decrease, following said administration, by at least 51% of an IGFBP2 level measured before first treatment with said inhibitor; or wherein the dosage is not selected if IGFBP2 levels are not observed to decrease, following said administration, by at least 51% of an IGFBP2 level measured before first treatment with said inhibitor.

41. The method of claim 39 wherein the IGF1R inhibitor is an antibody or antigen-binding fragment thereof that binds specifically to IGF1R.

42. The method of claim 41 wherein the antibody or fragment comprises one or more complementarity determining regions (CDRs) selected from the group consisting of:

| | |
|---|---|
| RASQSIGSSLH; | (SEQ ID NO:99) |
| YASQSLS; | (SEQ ID NO:100) |
| HQSSRLPHT; | (SEQ ID NO:101) |
| SFAMH | (SEQ ID NO:102) |
| GFTFSSFAMH; | (SEQ ID NO:107) |
| VIDTRGATYYADSVKG; | (SEQ ID NO:103) |
| and | |
| LGNFYYGMDV; | (SEQ ID NO:104) |

or a mature fragment of a light chain immunoglobulin which comprises the amino acid sequence of SEQ ID NO: 2, 4, 6 or 8; or a mature fragment of a heavy chain immunoglobulin which comprises the amino acid sequence of SEQ ID NO: 10 or 12; or a pharmaceutical composition thereof which comprises a pharmaceutically acceptable carrier.

43. The method of claim 39 wherein the disorder is a member selected from the group consisting of; osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

44. The method of claim 39 wherein the subject is also administered one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL 13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 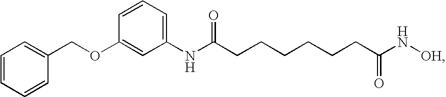
CG-781, CG-1521, 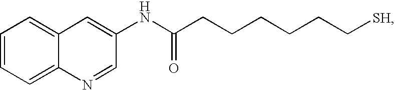
SB-556629, chlamydocin, JNJ-16241199, 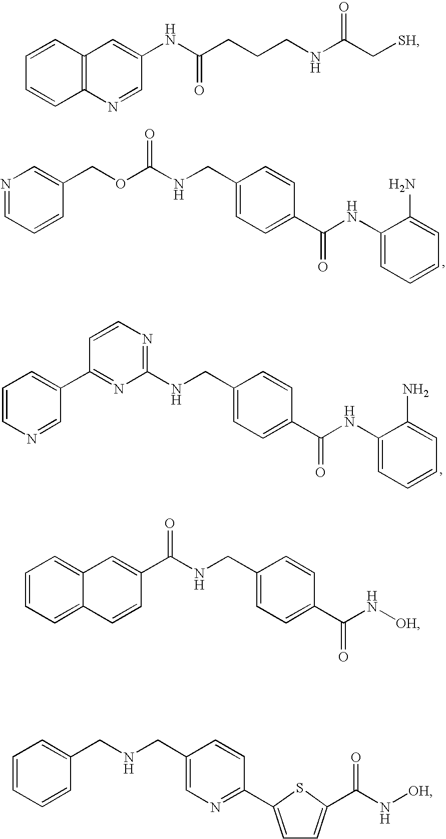
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES(diethylstilbestrol), estradiol, estrogen, conjugated estrogen, bevacizumab, IMC-1C11, CHIR-258, 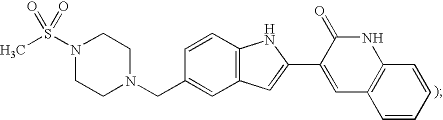
3-[5-(methylsulfonylpiperadinemethyl)-indolyl]-quinolone, vatalanib, AG-013736, AVE-0005 the acetate salt of [D-Ser(Bu t) 6, Azgly 10](pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 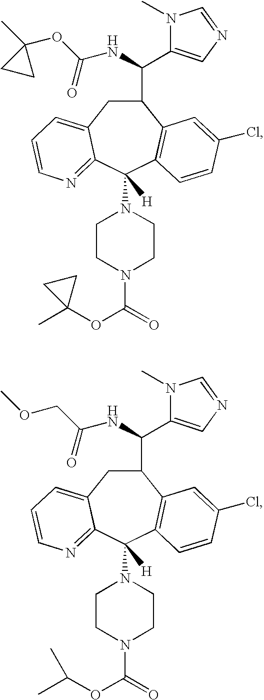
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene₁ ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.


SEQUENCE LISTING (107 sequences)

SEQ ID NO: 1 - DNA, 384 bp, Artificial Sequence
  light chain C
  CDS (1)..(384)
atgtcgccatcacaactcattgggtttctgctgctctgggttccagcctccaggggtgaaattgtgctgactcagagcccagactctctgtctgtgactccaggcgagagagtcaccatcacctgccgggccagtcagagcattggtagtagcttacactggtaccagcagaaaccaggtcagtctccaaagcttctcatcaagtatgcatcccagtccctctcaggggtcccctcgaggttcagtggcagtggatctgggacagatttcaccctcaccatcagtagcctcgaggctgaagatgctgcagcgtattactgtcatcagagtagtcgtttacctcacactttcggccaagggaccaaggtggagatcaaacgtacg

SEQ ID NO: 2 - protein, 128 aa, Artificial Sequence
  Synthetic Construct
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDAAAYYCHQSSRLPHTFGQGTKVEIKRT

SEQ ID NO: 3 - DNA, 384 bp, Artificial Sequence
  light chain D
  CDS (1)..(384)
atgtcgccatcacaactcattgggtttctgctgctctgggttccagcctccaggggtgaaattgtgctgactcagagcccagactctctgtctgtgactccaggcgagagagtcaccatcacctgccgggccagtcagagcattggtagtagcttacactggtaccagcagaaaccaggtcagtctccaaagcttctcatcaagtatgcatcccagtccctctcaggggtcccctcgaggttcagtggcagtggatctgggacagatttcaccctcaccatcagtagcctcgaggctgaagatttcgcagtgtattactgtcatcagagtagtcgtttacctcacactttcggccaagggaccaaggtggagatcaaacgtacg

SEQ ID NO: 4 - protein, 128 aa, Artificial Sequence
  Synthetic Construct
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDFAVYYCHQSSRLPHTFGQGTKVEIKRT

SEQ ID NO: 5 - DNA, 384 bp, Artificial Sequence
  light chain E
  CDS (1)..(384)
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatgctgcagcgtattactgtcatcagagtagtcgtttacctcacactttcggccaagggaccaaggtggagatcaaacgtaca

SEQ ID NO: 6 - protein, 128 aa, Artificial Sequence
  Synthetic Construct
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDAAAYYCHQSSRLPHTFGQGTKVEIKRT

SEQ ID NO: 7 - DNA, 384 bp, Artificial Sequence
  light chain F
  CDS (1)..(384)
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatttcgcagtgtattactgtcatcagagtagtcgtttacctcacactttcggccaagggaccaaggtggagatcaaacgtaca

SEQ ID NO: 8 - protein, 128 aa, Artificial Sequence
  Synthetic Construct
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 9 - DNA, 411 bp, Artificial Sequence
  heavy chain A
  CDS (1)..(411)
atggagtttgggctgagctgggttttccttgttgctatattaaaaggtgtccagtgtgaggttcagctggtgcagtctgggggaggcttggtaaagcctggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 10 - protein, 137 aa, Artificial Sequence
  Synthetic Construct
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 11 - DNA, 411 bp, Artificial Sequence
  heavy chain B
  CDS (1)..(411)
atggagtttgggctgagctgggttttccttgttgctatattaaaaggtgtccagtgtgaggttcagctggtgcagtctgggggaggcttggtacagcccggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 12 - protein, 137 aa, Artificial Sequence
  Synthetic Construct
MEFGLSWVFLVAILKGVQCEVQLVQSGGGLVQPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 13 - protein, 174 aa, Artificial Sequence
  immunoglobulin heavy chain variable region
GRLGQAWRSLRLSCAASGFTFSDYYMSWIRQAPGKGLEWVSYISSSGSTRDYADSVKGRFTISRDNAKNSLYLQMNSLRAEDTAVYYCVRDGVETTFYYYYYGMDVWGQGTTVTVSSASTKGPSVFPLAPCSRSTSESTAALGCLVKDYFPEPVTVSWNSGALTSGVHTFPSCA

SEQ ID NO: 14 - protein, 124 aa, Artificial Sequence
  immunoglobulin heavy chain variable region
VQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSS

SEQ ID NO: 15 - protein, 112 aa, Artificial Sequence
  immunoglobulin heavy chain variable region
GPGLVKPSETLSLTCTVSGGSISNYYWSWIRQPAGKGLEWIGRIYTSGSPNYNPSLKSRVTMSVDTSKNQFSLKLNSVTAADTAVYYCAVTIFGVVIIFDYWGQGTLVTVSS

SEQ ID NO: 16 - protein, 125 aa, Artificial Sequence
  immunoglobulin heavy chain variable region
EVQLLESGGGLVQPGGSLRLSCAASGFTFSSYAMSWVRQAPGKGLEWVSAISGSGGITYYADSVKGRFTISRDNSKNTLYLQMNSLRAEDTAVYYCAKDLGYGDFYYYYYGMDVWGQGTTVTVSS

SEQ ID NO: 17 - protein, 113 aa, Artificial Sequence
  immunoglobulin heavy chain variable region
PGLVKPSETLSLTCTVSGGSISSYYWSWIRQPPGKGLEWIGYIYYSGSTNYNPSLKSRVTISVDTSKNQFSLKLSSVTAADTAVYYCARTYSSSFYYYGMDVWGQGTTVTVSS

SEQ ID NO: 18 - protein, 122 aa, Artificial Sequence
  immunoglobulin heavy chain variable region
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 19 - protein, 136 aa, Artificial Sequence
  immunoglobulin light chain variable region
ASVGDRVTFTCRASQDIRRDLGWYQQKPGKAPKRLIYAASRLQSGVPSRFSGSGSGTEFTLTISSLQPEDFATYYCLQHNNYPRTFGQGTEVEIIRTVAAPSVFIFPPSDEQLKSGTASVVCLLNNFYPREAKVQW

SEQ ID NO: 20 - protein, 107 aa, Artificial Sequence [sequence omitted]

SEQ ID NO: 21 - protein, 100 aa, Artificial Sequence
  immunoglobulin light chain variable region
SSLSASVGDRVTFTCRASQDIRRDLGWYQQKPGKAPKRLIYAASRLQSGVPSRFSGSGSGTEFTLTISSLQPEDFATYYCLQHNNYPRTFGQGTEVEIIR

SEQ ID NO: 22 - protein, 107 aa, Artificial Sequence
  immunoglobulin light chain variable region
DIQMTQSPSSLSASVGDRVTITCRASQGIRSDLGWFQQKPGKAPKRLIYAASKLHRGVPSRFSGSGSGTEFTLTISRLQPEDFATYYCLQHNSYPLTFGGGTKVEIK

SEQ ID NO: 23 - protein, 92 aa, Artificial Sequence
  immunoglobulin light chain variable region
GDRVTITCRASQSISTFLNWYQQKPGKAPKLLIHVASSLQGGVPSRFSGSGSGTDFTLTISSLQPEDFATYYCQQSYNAPLTFGGGTKVEIK

SEQ ID NO: 24 - protein, 91 aa, Artificial Sequence
  immunoglobulin light chain variable region
RATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 25 - protein, 236 aa, Artificial Sequence
  light chain immunoglobulin
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 236 aa, Artificial Sequence
  light chain immunoglobulin
MDMRVPAQLLGLLLLWFPGARCDIQMTQSPSSLSASVGDRVTFTCRASQDIRRDLGWYQQKPGKAPKRLIYAASRLQSGVPSRFSGSGSGTEFTLTISSLQPEDFATYYCLQHNNYPRTFGQGTEVEIIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 236 aa, Artificial Sequence
  light chain immunoglobulin
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 236 aa, Artificial Sequence
  light chain immunoglobulin
MDMRVPAQLLGLLLLWFPGARCDIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 473 aa, Artificial Sequence
  heavy chain immunoglobulin
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVLRFLEWLLYYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 470 aa, Artificial Sequence
  heavy chain immunoglobulin
MEFGLSWVFLVAIIKGVQCQAQLVESGGGLVKPGGSLRLSCAASGFTFSDYYMSWIRQAPGKGLEWVSYISSSGSTRDYADSVKGRFTISRDNAKNSLYLQMNSLRAEDTAVYYCVRDGVETTFYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 470 aa, Artificial Sequence
  heavy chain immunoglobulin
MEFGLSWLFLVAILKGVQCEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSSGWYYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 470 aa, Artificial Sequence
  heavy chain immunoglobulin
MEFGLSWLFLVAILKGVQCEVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 470 aa, Artificial Sequence
  immunoglobulin heavy chain of 2.12.1 fx
MEFGLSWVFLVAIIKGVQCQVQLVESGGGLVKPGGSLRLSCAASGFTFSDYYMSWIRQAPGKGLEWVSYISSSGSTRDYADSVKGRFTISRDNAKNSLYLQMNSLRAEDTAVYYCARDGVETTFYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 125 aa, Artificial Sequence
  mature immunoglobulin heavy chain variable region of 2.12.1 fx
QVQLVESGGGLVKPGGSLRLSCAASGFTFSDYYMSWIRQAPGKGLEWVSYISSSGSTRDYADSVKGRFTISRDNAKNSLYLQMNSLRAEDTAVYYCARDGVETTFYYYYYGMDVWGQGTTVTVSS

SEQ ID NO: 35 - protein, 236 aa, Artificial Sequence
  immunoglobulin light chain of 2.12.1 fx
MDMRVPAQLLGLLLLWFPGARCDIQMTQSPSSLSASVGDRVTITCRASQDIRRDLGWYQQKPGKAPKRLIYAASRLQSGVPSRFSGSGSGTEFTLTISSLQPEDFATYYCLQHNNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 108 aa, Artificial Sequence
  mature immunoglobulin light chain variable region of 2.12.1 fx
DIQMTQSPSSLSASVGDRVTITCRASQDIRRDLGWYQQKPGKAPKRLIYAASRLQSGVPSRFSGSGSGTEFTLTISSLQPEDFATYYCLQHNNYPRTFGQGTKVEIKR

SEQ ID NO: 37 - protein, 112 aa, Artificial Sequence
  humanized 7C10 immunoglobulin light chain variable region; version 1
DVVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIK

SEQ ID NO: 38 - protein, 112 aa, Artificial Sequence [sequence omitted]

SEQ ID NO: 39 - protein, 117 aa, Artificial Sequence
  humanized 7C10 immunoglobulin heavy chain variable region; version 1
QVQLQESGPGLVKPSETLSLTCTVSGYSITGGYLWNWIRQPPGKGLEWMGYISYDGTNNYKPSLKDRITISRDTSKNQFSLKLSSVTAADTAVYYCARYGRVFFDYWGQGTLVTVSS

SEQ ID NO: 40 - protein, 117 aa, Artificial Sequence [sequence omitted]

SEQ ID NO: 41 - protein, 117 aa, Artificial Sequence
  humanized 7C10 immunoglobulin heavy chain variable region; version 3
QVQLQESGPGLVKPSETLSLTCTVSGYSISGGYLWNWIRQPPGKGLEWIGYISYDGTNNYKPSLKDRVTISVDTSKNQFSLKLSSVTAADTAVYYCARYGRVFFDYWGQGTLVTVSS

SEQ ID NO: 42 - protein, 130 aa, Artificial Sequence
  A12 immunoglobulin heavy chain variable region
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 43 - protein, 109 aa, Artificial Sequence
  A12 immunoglobulin light chain variable region
SSELTQDPAVSVALGQTVRITCQGDSLRSYYASWYQQKPGQAPVLVIYGKNNRPSGIPDRFSGSSSGNTASLTITGAQAEDEADYYCNSRDNSDNRLIFGGGTKLTVLS

SEQ ID NO: 44 - protein, 119 aa, Artificial Sequence
  1A immunoglobulin heavy chain variable region
  MISC_FEATURE (1)..(119): Possible mutations R30, S30, N31, S31, Y94, H94, D104, E104.
EVQLVQSGGGLVHPGGSLRLSCAGSGFTFRNYAMYWVRQAPGKGLEWVSAIGSGGGTYYADSVKGRFTISRDNAKNSLYLQMNSLRAEDMAVYYCARAPNWGSDAFDIWGQGTMVTVSS

SEQ ID NO: 45 - protein, 107 aa, Artificial Sequence
  1A immunoglobulin light chain variable region
  MISC_FEATURE (1)..(107): possible mutations P96, I96, P100, Q100, R103, K103, V104, L104, D105, E105
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGPGTKVDIK

SEQ ID NO: 46 - protein, 251 aa, Artificial Sequence
  single chain fv 8A1
EVQLVQSGAEVKKPGESLTISCKGPGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTMVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 47 - protein, 245 aa, Artificial Sequence
  single chain fv 9A2
QVQLVQSGAEVRKPGASVKVSCKTSGYTFRNYDINWVRQAPGQGLEWMGRISGHYGNTDHAQKFQGRFTMTKDTSTSTAYMELRSLTFDDTAVYYCARSQWNVDYWGRGTLVTVSSGGGGSGGGGSGGGGSALNFMLTQPHSVSESPGKTVTISCTRSSGSIASNYVQWYQQRPGSSPTTVIFEDNRRPSGVPDRFSGSIDTSSNSASLTISGLKTEDEADYYCQSFDSTNLVVFGGGTKVTVLG

SEQ ID NO: 48 - protein, 245 aa, Artificial Sequence
  single chain fv 11A4
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCASSPYSSRWYSFDPWGQGTMVTVSSGGGGSGGGGSGGGGSALSYELTQPPSVSVSPGQTATITCSGDDLGNKYVSWYQQKPGQSPVLVIYQDTKRPSGIPERFSGSNSGNIATLTISGTQAVDEADYYCQVWDTGTVVFGGGTKLTVLG

SEQ ID NO: 49 - protein, 251 aa, Artificial Sequence
  single chain fv 7A4
EVQLVQSGAEVKKPGESLTISCKGSGYNFFNYWIGWVRQMPGKDLEWMGIIYPTDSDTRYSPSFQGQVTISVDKSISTAYLQWSSLKASDTAMYYCARSIRYCPGGRCYSGYYGMDVWGQGTMVTVSSGGGSSGGGGSGGGGSSELTQDPAVSVALGQTVRITCRGDSLRNYYASWYQQKPGQAPVLVIYGKNNRPSGIPDRFSGSSSGNTASLTITGAQAEDEADYYCNSRDSSGNHMVFGGGTKLTVLG

SEQ ID NO: 50 - protein, 249 aa, Artificial Sequence
  single chain fv 11A1
EVQLVESGGGVVQPGRSLRLSCAASGFTFSDFAMHWVRQIPGKGLEWLSGLRHDGSTAYYAGSVKGRFTISRDNSRNTVYLQMNSLRAEDTATYYCVTGSGSSGPHAFPVWGKGTLVTVSSGGGGSGGGGSGGGGSALSYVLTQPPSASGTPGQRVTISCSGSNSNIGTYTVNWFQQLPGTAPKLLIYSNNQRPSGVPDRFSGSKSGTSASLAISGLQSEDEADYYCAAWDDSLNGPVFGGGTKVTVLG

SEQ ID NO: 51 - protein, 251 aa, Artificial Sequence
  single chain fv 7A6
EVQLVQSGAEVKKPGESLTISCKGSGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTLVTVSSGGGGSGGGGSGGGGSSELTQDPAVSVALGQTVRITCQGDSLRSYYTNWFQQKPGQAPLLVVYAKNKRPSGIPDRFSGSSSGNTASLTITGAQAEDEADYYCNSRDSSGNHVVFGGGTKLTVLG

SEQ ID NO: 52 - protein, 5 aa, Artificial Sequence
  CDR
SYWMH

SEQ ID NO: 53 - protein, 17 aa, Artificial Sequece
  CDR
EINPSNGRTNYNEKFKR

SEQ ID NO: 54 - protein, 15 aa, Artificial Sequence
  CDR
GRPDYYGSSKWYFDV

SEQ ID NO: 55 - protein, 16 aa, Artificial Sequence
  CDR
RSSQSIVHSNVNTYLE

SEQ ID NO: 56 - protein, 7 aa, Artificial Sequence
  CDR
KVSNRFS

SEQ ID NO: 57 - protein, 9 aa, Artificial Sequence
  CDR
FQGSHVPPT

SEQ ID NO: 58 - protein, 123 aa, Artificial Sequence
  heavy chain immunoglobulin variable region
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVS

SEQ ID NO: 59 - protein, 118 aa, Artificial Sequence
  heavy chain immunoglobulin variable region
QVQFQQSGAELVKPGASVKLSCKASGYTFTSYLMHWIKQRPGRGLEWIGRIDPNNVVTKFNEKFKSKATLTVDKPSSTAYMELSSLTSEDSAVYYCARYAYCRPMDYWGQGTTVTVSS

SEQ ID NO: 60 - protein, 123 aa, Artificial Sequence
  heavy chain immunoglobulin variable region
QVQLQQSGAELVKPGASVKLSCKASGYTFTSYWMHWVKQRPGQGLEWIGEINPSNGRTNYNEKFKRKATLTVDKSSSTAYMQLSSLTSEDSAVYYFARGRPDYYGSSKWYFDVWGAGTTVTVS

SEQ ID NO: 61 - protein, 120 aa, Artificial Sequence
  heavy chain immunoglobulin variable region
QVQLQQSGAELMKPGASVKISCKATGYTFSSFWIEWVKQRPGHGLEWIGEILPGSGGTHYNEKFKGKATFTADKSSNTAYMQLSSLTSEDSAVYYCARGHSYYFYDGDYWGQGTSVTVSS

SEQ ID NO: 62 - protein, 120 aa, Artificial Sequence
  heavy chain immunoglobulin variable region
QVQLQQPGSVLVRPGASVKLSCKASGYTFTSSWIHWAKQRPGQGLEWIGEIHPNSGNTNYNEKFKGKATLTVDTSSSTAYVDLSSLTSEDSAVYYCARWRYGSPYYFDYWGQGTTLTVSS

SEQ ID NO: 63 - protein, 120 aa, Artificial Sequence
  heavy chain immunoglobulin variable region
QVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARYDYYGSSYFDYWGQGTTLTVSS

SEQ ID NO: 64 - protein, 123 aa, Artificial Sequence; identical to SEQ ID NO: 58
  heavy chain immunoglobulin variable region

SEQ ID NO: 65 - protein, 124 aa, Artificial Sequence
  heavy chain immunoglobulin variable region
QVQLQQSGAELVKPGASVKLSCKASGYTFTSYWMHWVKQRPGQGLEWIGEINPSNGRTNYNEKFKRKATLTVDKSSSTAYMQLSSLTSEDSAVYYFARGRPDYYGSSKWYFDVWGAGTTVTVSS

SEQ ID NO: 66 - protein, 124 aa, Artificial Sequence
  heavy chain immunoglobulin variable region
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 67 - protein, 120 aa, Artificial Sequence
  heavy chain immunoglobulin variable region
QVQLQQSGAELVKPGASVKLSCKASGYTFTSYWMHWVKQRPGRGLEWIGRIDPNSGGTKYNEKFKSKATLTVDKPSSTAYMQLSSLTSEDSAVYYCARYDYYGSSYFDYWGQGTTVTVSS

SEQ ID NO: 68 - protein, 117 aa, Artificial Sequence
  heavy chain immunoglobulin variable region
QIQLQQSGPELVRPGASVKISCKASGYTFTDYYIHWVKQRPGEGLEWIGWIYPGSGNTKYNEKFKGKATLTVDTSSSTAYMQLSSLTSEDSAVYFCARGGKFAMDYWGQGTSVTVSS

SEQ ID NO: 69 - protein, 124 aa, Artificial Sequence; identical to SEQ ID NO: 65
  heavy chain immunoglobulin variable region

SEQ ID NO: 70 - protein, 120 aa, Artificial Sequence
  heavy chain immunoglobulin variable region
QIQLQQSGPELVKPGASVKISCKASGYTFTDYYINWMKQKPGQGLEWIGWIDPGSGNTKYNEKFKGKATLTVDTSSSTAYMQLSSLTSEDTAVYFCAREKTTYYYAMDYWGQGTSVTVSA

SEQ ID NO: 71 - protein, 115 aa, Artificial Sequence
  heavy chain immunoglobulin variable region
VQLQQSGAELMKPGASVKISCKASGYTFSDYWIEWVKQRPGHGLEWIGEILPGSGSTNYHERFKGKATFTADTSSSTAYMQLNSLTSEDSGVYYCLHGNYDFDGWGQGTTLTVSS

SEQ ID NO: 72 - protein, 120 aa, Artificial Sequence
  heavy chain immunoglobulin variable region
QVQLLESGAELMKPGASVKISCKATGYTFSSFWIEWVKQRPGHGLEWIGEILPGSGGTHYNEKFKGKATFTADKSSNTAYMQLSSLTSEDSAVYYCARGHSYYFYDGDYWGQGTSVTVSS

SEQ ID NO: 73 - protein, 113 aa, Artificial Sequence
  light  chain immunoglobulin variable region
DVLMTQIPVSLPVSLGDQASISCRSSQIIVHNNGNTYLEWYLQKPGQSPQLLIYKVSNRFSGVPDRFSGSGSGTDFTLKISRVEAEDLGVYYCFQGSHVPFTFGSGTKLEIKR

SEQ ID NO: 74 - protein, 113 aa, Artificial Sequence
  light chain immunoglobulin variable region
DVLMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 75 - protein, 113 aa, Artificial Sequence
  light chain immunoglobulin variable region
DVLMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 76 - protein, 113 aa, Artificial Sequence; identical to SEQ ID NO: 74
  light chain immunoglobulin variable region

SEQ ID NO: 77 - protein, 113 aa, Artificial Sequence; identical to SEQ ID NO: 75
  light chain immunoglobulin variable region

SEQ ID NO: 78 - protein, 113 aa, Artificial Sequence
  light chain immunoglobulin variable region
  misc_feature (28)..(28): Xaa can be any naturally occurring amino acid
  misc_feature (101)..(101): Xaa can be any naturally occurring amino acid
DVLMTQTPLSLPVSLGDQASISCRSSQXIVHSNGNTYLEWYLQKPGQSPKLLIYKVSNRFSGVPDRFSGSGSGTDFTLKISRVEAEDLGVYYCFQGSHVPXTFGGGTKLEIKR

SEQ ID NO: 79 - protein, 113 aa, Artificial Sequence
  light chain immunoglobulin variable region
DVVMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 80 - protein, 113 aa, Artificial Sequence
  light chain immunoglobulin variable region
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 81 - protein, 113 aa, Artificial Sequence; identical to SEQ ID NO: 75
  light chain immunoglobulin variable region

SEQ ID NO: 82 - protein, 113 aa, Artificial Sequence; identical to SEQ ID NO: 73
  light chain immunoglobulin variable region

SEQ ID NO: 83 - protein, 113 aa, Artificial Sequence
  light chain immunoglobulin variable region
DVLMTQTPLSLPVSLGDQASISCRFSQSIVHSNGNTYLEWYLQKSGQSPKLLIYKVSNRFSGVPDRFSGSGSGTDFTLKISRVEAEDLGVYYCFQGSHVPRTFGGGTKLEIKR

SEQ ID NO: 84 - protein, 113 aa, Artificial Sequence
  light chain immunoglobulin variable region
DVLMTQTPLSLPVSLGDQASISCRSSQSIVHSNVNTYLEWYLQKPGQSPKLLIYKVSNRFSGVPDRFSGSGSGTDFTLRISRVEAEDLGIYYCFQGSHVPPTFGGGTKLEIKR

SEQ ID NO: 85 - protein, 113 aa, Artificial Sequence; identical to SEQ ID NO: 79
  light chain immunoglobulin variable region

SEQ ID NO: 86 - protein, 113 aa, Artificial Sequence
  light chain immunoglobulin variable region
ELVMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPPTFGGGTKLEIKR

SEQ ID NO: 87 - protein, 113 aa, Artificial Sequence; identical to SEQ ID NO: 74
  light chain immunoglobulin variable region

SEQ ID NO: 88 - protein, 113 aa, Artificial Sequence; identical to SEQ ID NO: 80
  light chain immunoglobulin variable region

SEQ ID NO: 89 - protein, 113 aa, Artificial Sequence
  light chain immunoglobulin variable region
DVLMTQTPVSLSVSLGDQASISCRSSQSIVHSTGNTYLEWYLQKPGQSPKLLIYKISNRFSGVPDRFSGSGSGTDFTLKISRVEAEDLGVYYCFQASHAPRTFGGGTKLEIKR

SEQ ID NO: 90 - protein, 113 aa, Artificial Sequence
  light chain immunoglobulin variable region
DVLMTQTPLSLPVSLGDQASISCKSSQSIVHSSGNTYFEWYLQKPGQSPKLLIYKVSNRFSGVPDRFSGSGSGTDFTLKISRVEAEDLGVYYCFQGSHIPFTFGSGTKLEIKR

SEQ ID NO: 91 - protein, 113 aa, Artificial Sequence
  light chain immunoglobulin variable region
DIELTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKR

SEQ ID NO: 92 - protein, 113 aa, Artificial Sequence; identical to SEQ ID NO: 84
  light chain immunoglobulin variable region

SEQ ID NO: 93 - protein, 113 aa, Artificial Sequence; identical to SEQ ID NO: 80
  light chain immunoglobulin variable region

SEQ ID NO: 94 - protein, 113 aa, Artificial Sequence; identical to SEQ ID NO: 84
  light chain immunoglobulin variable region

SEQ ID NO: 95 - protein, 113 aa, Artificial Sequence; identical to SEQ ID NO: 79
  light chain immunoglobulin variable region

SEQ ID NO: 96 - protein, 113 aa, Artificial Sequence
  light chain immunoglobulin variable region
DVLMTQTPLSLPVSLGDQASISCRSNQTILLSDGDTYLEWYLQKPGQSPKLLIYKVSNRFSGVPDRFSGSGSGTDFTLKISRVEAEDLGVYYCFQGSHVPPTFGGGTKLEIKR

SEQ ID NO: 97 - protein, 113 aa, Artificial Sequence
  light chain immunoglobulin variable region
DVLMTQTPLSLPVSLGDQASISCRSSQTIVHSNGNTYLEWYLQKPGQSPKLLIYKVTNRFSGVPDRFSGSGSGTDFTLKISRVEAEDLGVYYCFQGTHAPYTFGGGTKLEIKR

SEQ ID NO: 98 - protein, 113 aa, Artificial Sequence
  light chain immunoglobulin variable region
DVLMTQTPLSLPVSLGDQASISCRSSQSIVHSNGNTYLEWYLQKPGQSPKLLIYSISSRFSGVPDRFSGSGSGTDFTLKISRVQAEDLGVYYCFQGSHVPYTFGGGTKLEIKR

SEQ ID NO: 99 - protein, 11 aa, Artificial Sequence
  CDR
RASQSIGSSLH

SEQ ID NO: 100 - protein, 7 aa, Artificial Sequence
  CDR
YASQSLS

SEQ ID NO: 101 - protein, 9 aa, Artificial Sequence
  CDR
HQSSRLPHT

SEQ ID NO: 102 - protein, 5 aa, Artificial Sequence
  CDR
SFAMH

SEQ ID NO: 103 - protein, 16 aa, Artificial Sequence
  CDR
VIDTRGATYYADSVKG

SEQ ID NO: 104 - protein, 10 aa, Artificial Sequence
  CDR
LGNFYYGMDV

SEQ ID NO: 105 - protein, 328 aa, Homo Sapiens
MLPRVGCPALPLPPPPLLPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 106 - protein, 109 aa, Artificial Sequence
  A12 immunoglobulin light chain variable region
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLG

SEQ ID NO: 107 - protein, 10 aa, Artificial sequence
  heavy chain immunoglobulin variable region
GFTFSSFAMH